(19) United States
(12) Patent Application Publication
(10) Pub. No.: US 2017/0137535 A1
(43) Pub. Date: May 18, 2017

(54) ANTI-FACTOR D ANTIBODY FORMULATIONS

(71) Applicant: Genentech, Inc., South San Francisco, CA (US)

(72) Inventors: Christopher Petry, South San Francisco, CA (US); Benson Gikanga, South San Francisco, CA (US); Hung-Wei Chih, South San Francisco, CA (US); Jun Liu, South San Francisco, CA (US)

(21) Appl. No.: 15/336,578

(22) Filed: Oct. 27, 2016

Related U.S. Application Data
(60) Provisional application No. 62/249,082, filed on Oct. 30, 2015.
(60) Provisional application No. 62/251,015, filed on Nov. 4, 2015.

(51) Int. Cl. C07K 16/40; A61K 9/00; A61K 47/26; A61K 47/22; A61K 39/395; A61K 47/18

(52) U.S. Cl. CPC C07K 16/40; A61K 39/3955; A61K 47/183; A61K 47/26; A61K 47/22; A61K 9/0048; C07K 2317/55; C07K 2317/24

(57) ABSTRACT

Pharmaceutical formulations comprising monoclonal anti-Factor D antibodies, and their production and use for the treatment of complement-associated ocular diseases are disclosed. The formulations include pre-lyophilized, lyophilized and reconstituted stable liquid formulations of anti-Factor D antibodies, including lampalizumab.

### CROSS-REFERENCE TO RELATED APPLICATIONS

This application claims priority under 35 USC Section 119(e) and the benefit of U.S. Provisional Application No. 62/249,082, filed Oct. 30, 2015, and Provisional Application No. 62/251,015, filed Nov. 4, 2015, the entire disclosures of which are incorporated herein by reference.

### SEQUENCE LISTING

The instant application contains a Sequence Listing which has been submitted electronically in ASCII format and is hereby incorporated by reference in its entirety. Said ASCII copy, created on Oct. 5, 2016, is named GNE0419US_SL.txt and is 66,252 bytes in size.

### FIELD OF THE INVENTION

The present invention concerns anti-Factor D antibody formulations. In particular, the invention concerns pre-lyophilized, lyophilized and reconstituted stable liquid formulations of anti-Factor D antibodies, suitable for intravitreal administration.

### BACKGROUND OF THE INVENTION

Age Related Macular Degeneration (AMD)

The complement system plays a central role in the clearance of immune complexes and the immune response to infectious agents, foreign antigens, virus-infected cells and tumor cells. However, complement is also involved in pathological inflammation and in autoimmune diseases. Therefore, inhibition of excessive or uncontrolled activation of the complement cascade could provide clinical benefit to patients with such diseases and conditions.

The complement system encompasses three distinct activation pathways, designated the classical, mannose-binding lectin and the alternative pathways (V. M. Holers In Clinical Immunology: Principles and Practice, ed. R. R. Rich, Mosby Press; 1996, 363-391). The classical pathway is a calcium/magnesium-dependent cascade which is normally activated by the formation of antigen-antibody complexes. The mannose-binding lectin (MBL) pathway is initiated by the binding of MBL to carbohydrate structures on pathogens, resulting in the activation of MBL protease (MASP) that cleaves C2 and C4 to form active C2a, C2b, C4a and C4b. The alternative pathway is a magnesium-dependent cascade which is activated by deposition and activation of C3 on certain susceptible surfaces (e.g. cell wall polysaccharides of yeast and bacteria, and certain biopolymer materials). Activation of the complement pathway generates biologically active fragments of complement proteins, e.g. C3a, C4a and C5a anaphylatoxins and C5b-9 membrane attack complexes (MAC), which mediate inflammatory activities involving leukocyte chemotaxis, activation of macrophages, neutrophils, platelets, mast cells and endothelial cells, vascular permeability, cytolysis, and tissue injury.

Factor D is a highly specific serine protease essential for activation of the alternative complement pathway. It cleaves factor B bound to C3b, generating the C3b/Bb enzyme which is the active component of the alternative pathway C3/C5 convertases. Factor D may be a suitable target for inhibition, since its plasma concentration in humans is very low (1.8 μg/ml), and it has been shown to be the limiting enzyme for activation of the alternative complement pathway (P. H. Lesavre and H. J. Müller-Eberhard. (1978) J. Exp. Med. 148: 1498-1510; J. E. Volanakis et al. (1985) New Eng. J. Med. 312: 395-401).

The down-regulation of complement activation has been demonstrated to be effective in treating several disease indications in animal models and in ex vivo studies, e.g. systemic lupus erythematosus and glomerulonephritis, rheumatoid arthritis, cardiopulmonary bypass and hemodialysis, hyperacute rejection in organ transplantation, myocardial infarction, reperfusion injury, and adult respiratory distress syndrome. In addition, other inflammatory conditions and autoimmune/immune complex diseases are also closely associated with complement activation, including thermal injury, severe asthma, anaphylactic shock, bowel inflammation, urticaria, angioedema, vasculitis, multiple sclerosis, myasthenia gravis, membranoproliferative glomerulonephritis, and Sjögren's syndrome.

Age-related macular degeneration (AMD) is a progressive chronic disease of the central retina with significant consequences for visual acuity. Lim et al. (2012) Lancet 379:1728. Late forms of the disease are the leading cause of vision loss in industrialized countries. For the Caucasian population ≧40 years of age the prevalence of early AMD is estimated at 6.8% and advanced AMD at 1.5%. de Jong (2006) N Engl. J. Med. 355: 1474. The prevalence of late AMD increases dramatically with age rising to 11.8% after 80 years of age. Two types of AMD exist, non-exudative (dry) and exudative (wet) AMD. The more common dry form AMD involves atrophic and hypertrophic changes in the retinal pigment epithelium (RPE) underlying the central retina (macula) as well as deposits (drusen) on the RPE. Advanced dry AMD can result in significant retinal damage, including geographic atrophy (GA), with irreversible vision loss. Moreover, patients with dry AMD can progress to the wet form, in which abnormal blood vessels called choroidal neovascular membranes (CNVMs) develop under the retina, leak fluid and blood, and ultimately cause a blinding disciform scar in and under the retina.

Drugs targeting new blood vessel formation (neovascularization) have been the mainstay for treating wet AMD. Ranibizumab, which is an anti-VEGFA antibody fragment, has proven to be highly effective in improving vision for patients afflicted with wet AMD. Recent studies have implicated an association between AMD and key proteins in the complement cascade and a number of therapies targeting specific complement components are being developed to treat dry AMD.

Treatment of AMD with Anti-Factor D Antibodies

Humanized anti-Factor D antibodies are disclosed, for example, in U.S. Pat. No. 8,273,352. A humanized anti-Factor D Fab fragment (aFD.WT, lampalizumab; FCFD4514S) that potently inhibits Factor D and the alternative complement pathway, through binding to an exosite on factor D is currently in clinical development for the treatment of GA associated with dry AMD. Katschke et al. (2012) J. Biol. Chem. 287:12886. A recent phase II clinical trial has shown that monthly intravitreal injection of lampalizumab effectively slowed the progression of GA lesions in patients with advanced dry AMD. Two Phase III clinical trials (GX29176 and GX29185) investigating the efficacy and safety of lampalizumab intravitreal injections in patients with Geographic Atrophy (GA) secondary to AMD are under way.

Formulations for Intravitreal Administration

Drug administration for the treatment of retinal diseases is very challenging. The anatomical features of the eye present multiple barriers to any foreign substance, including the blood-retinal barrier, and the blood aqueous barrier (Duvvuri S, et al., Expert Opin Biol Ther. 2003; 3(1):45-56). Such blood-ocular barriers are defense mechanisms for protecting the eye from infection, but also make it hard for drugs to penetrate, especially for diseases in the posterior segments of the eye. Consequently, the drug levels achievable relative to other delivery routes, such as topical delivery to the eye, are limited, and high-dose administration is often desired to achieve and maintain a drug's onsite bioavailability (e.g., ocular residence time) in order to improve efficacy. In general, invasive drug delivery strategies requiring injection directly into the vitreous (intravitreal delivery route) are needed to deliver drugs to the retina.

However, the intravitreal injection route presents several unique formulation challenges. The eye is an extremely sensitive organ, and there is a limited collection of excipients acceptable for intravitreal injection compared with other delivery routes. As intravitreal injection is an invasive route, there is always a small but significant risk of infection with each new injection, thus, there is a drive to minimize the injection frequency (Duvvury et al., supra; Urtti A. et al., Adv Drug Deliv Rev. 2006; 58(11):1131-11351; Ghate D, et al., Expert Opin Drug Deliv. 2006; 3(2):275-287).

All these constraints present challenges that are not easily overcome. Low dosing volumes (≦0.1 mL), a limited repertoire of safe excipients for intravitreal injection, and the unique physical chemical properties of the drug to be delivered must be addressed. In addition, safety considerations associated with intravitreal administration place constraints on the osmolality and pH of formulations, that, coupled with stability issues, makes formulation of anti-Factor D antibodies for intravitreal use particularly challenging. Stability issues associated with monoclonal antibody Fab fragments, including isomerization and racemization of aspartate in Asp-Asp motifs, are discussed, for example, in Wang et al., J Pharmaceutical Sci 2013; 102(8):2520-2537; Beckley et al., J Pharmaceutical Sci 2013; 102(3):947-959; and Zhang et al., Analytical Biochemistry 2011; 410:234-243.

Lampalizumab is currently in phase III clinical trials for treatment of geographic atrophy (GA), an advanced form of dry AMD. The Phase I/II lampalizumab Drug Product (DP) was formulated as 100 mg/mL lampalizumab in 40 mM L-histidine/L-histidine hydrochloride (histidine chloride, HisCl), 20 mM sodium chloride (NaCl), 180 mM sucrose, and 0.04% PS20 at pH 5.5 after reconstitution. During development, it was observed that the solubility of lampalizumab in the Phase I/II DP formulation buffer was not satisfactory for further clinical development. In order to develop an anti-Factor D formulation with improved solubility while maintaining suitable sugar-to-protein ratio to minimize soluble aggregate formation in the solid state and tonicity that is appropriate for intravitreal administration, alternative anti-Factor D formulations have been investigated.

### SUMMARY OF THE INVENTION

The present invention is based, at least in part, on the development of anti-Factor D antibody formulations that provide for improved solubility of the anti-Factor D antibody while retaining stability of the antibody molecule during storage.

In one aspect, the present invention concerns a pharmaceutical formulation comprising a therapeutically effective amount of a monoclonal anti-Factor D antibody, a buffer adjusting the pH to between 5.0 and 5.4, a lyoprotectant and a surfactant.

In some embodiments, the pH of the formulation is about 5.3.

In some embodiments, the lyoprotectant to antibody ratio in the formulation is about 60 to 100 mole lyoprotectant:1 mole antibody, preferably about 80 mole lyoprotectant:1 mole antibody.

In some embodiments, the buffer used to adjust the pH of the formulation is a histidine buffer, which may, for example, be present in an amount of about 5 mM to about 15 mM, or in an amount of about 7 mM to about 13 mM.

In some embodiments, the lyoprotectant present in the formulation comprises one or more polyols.

In some embodiments, at least one of the polyols is a reducing sugar, such as, for example, α,α-trehalose, or a non-reducing sugar, such, as for example, sucrose.

In some embodiments, at least one of the polyols is a disaccharide.

In some embodiments, the surfactant present in the formulation comprises one or more polysorbates, e.g. polysorbate 20, and/or poloxamers.

In some embodiments, the monoclonal anti-Factor D antibody present in the formulation comprises heavy chain hypervariable regions (HVR-HCs) having at least 98% or at least 99% sequence identity to the HVR sequences of HVR1-HC: GYTFTNYGMN (SEQ ID NO: 3); HVR2-HC: WINTYTGETTYADDFKG (SEQ ID NO: 4); HVR3-HC: EGGVNN (SEQ ID NO: 5) and/or light chain hypervariable regions (HVR-LCs) having at least 98% or at least 99% sequence identity to the HVR-LC sequences of HVR1-LC: ITSTDIDDDMN (SEQ ID NO: 8); HVR2-LC: GGNTLRP (SEQ ID NO: 9); and HVR3-LC: LQSDSLPYT (SEQ ID NO: 10).

In some embodiments, the monoclonal anti-Factor D antibody comprises the HVR-HCs of HVR1-HC: GYTFTNYGMN (SEQ ID NO: 3); HVR2-HC: WINTYTGETTYADDFKG (SEQ ID NO: 4); HVR3-HC: EGGVNN (SEQ ID NO: 5) and/or the HVR-LC of HVR1-LC: ITSTDIDDDMN (SEQ ID NO: 8); HVR2-LC: GGNTLRP (SEQ ID NO: 9); and HVR3-LC: LQSDSLPYT (SEQ ID NO: 10).

In some embodiments, the monoclonal anti-Factor D antibody comprises a heavy chain variable region sequence having at least 85%, or at least 90%, or at least 95%, or at least 98%, or at least 99% sequence identity to the variable region sequence of the heavy chain of SEQ ID NO: 2 and/or a light chain variable region sequence having at least 85%, or at least 90%, or at least 95%, or at least 98%, or at least 99% sequence identity to the variable region sequence of the light chain of SEQ ID NO: 7.

In some embodiments, the monoclonal anti-Factor D antibody comprises the variable region sequence of the heavy chain of SEQ ID NO: 2 and/or the variable region sequence of the light chain of SEQ ID NO: 7.

In some embodiments, the monoclonal anti-Factor D antibody comprises a heavy chain sequence comprising SEQ ID NO: 2 and/or a light chain sequence comprising SEQ ID NO: 7.

In some embodiments, the monoclonal anti-Factor D antibody is an IgG antibody, such as an IgG1 antibody.

In some embodiments, the monoclonal anti-Factor D antibody is an antibody fragment, such as a Fab fragment.

In some embodiments, the monoclonal anti-Factor D antibody is humanized.

In some embodiments, the monoclonal anti-Factor D antibody is lampalizumab.

The pharmaceutical formulations herein may, for example, be for intraocular administration, including intravitreal administration.

In various embodiments, the pharmaceutical formulations herein may be sterile and/or stable upon freezing and thawing.

In some embodiments, the pharmaceutical formulation is a pre-lyophilized formulation.

In some embodiments, the pre-lyophilized formulation is stable at a storage temperature of −20° C. for at least one year, or for at least two years.

In some embodiments, the pharmaceutical formulation is lyophilized.

In some embodiments, the lyophilized pharmaceutical formulation is stable at a storage temperature of 5° C. for at least one year, or for at least two years.

In another aspect, the invention concerns a reconstituted aqueous liquid formulation prepared from any of the pharmaceutical formulations hereinabove described or otherwise disclosed.

In yet another aspect, the invention concerns a pre-lyophilized or lyophilized pharmaceutical formulation comprising a therapeutically effective amount of a monoclonal anti-Factor D antibody, about 5 mM to about 15 mM of a histidine buffer adjusting the pH to between 5.0 and 5.4, sodium chloride, a lyoprotectant and a surfactant.

In some embodiments, the anti-Factor D antibody present in the pre-lyophilized or lyophilized pharmaceutical formulation comprises heavy chain hypervariable regions (HVR-HCs) having at least 98% or at least 99% sequence identity to the HVR sequences of HVR1-HC: GYTFTNYGMN (SEQ ID NO: 3); HVR2-HC: WINTYTGETTYADDFKG (SEQ ID NO: 4); HVR3-HC: EGGVNN (SEQ ID NO: 5) and/or light chain hypervariable regions (HVR-LCs) having at least 98% or at least 99% sequence identity to the HVR-LC sequences of HVR1-LC: ITSTDIDDDMN (SEQ ID NO: 8); HVR2-LC: GGNTLRP (SEQ ID NO: 9); and HVR3-LC: LQSDSLPYT (SEQ ID NO: 10).

In some embodiments, the monoclonal anti-Factor D antibody comprises the heavy chain hypervariable regions (HVR-HCs) of HVR1-HC: GYTFTNYGMN (SEQ ID NO: 3); HVR2-HC: WINTYTGETTYADDFKG (SEQ ID NO: 4); HVR3-HC: EGGVNN (SEQ ID NO: 5) and/or the light chain hypervariable regions (HVR-LCs) of HVR1-LC: ITSTDIDDDMN (SEQ ID NO: 8); HVR2-LC: GGNTLRP (SEQ ID NO: 9); and HVR3-LC: LQSDSLPYT (SEQ ID NO: 10).

In some embodiments, the monoclonal anti-Factor D antibody comprises a heavy chain variable region sequence having at least 85%, or at least 90%, or at least 95%, or at least 98%, or at least 99% sequence identity to the heavy chain of SEQ ID NO: 2 and/or a light chain variable region sequence having at least 85%, or at least 90%, or at least 95%, or at least 98%, or at least 99% sequence identity to the light chain of SEQ ID NO: 7.

In some embodiments, the monoclonal anti-Factor D antibody comprises a heavy chain sequence comprising SEQ ID NO: 2 and/or a light chain sequence comprising SEQ ID NO: 7.

The monoclonal anti-Factor D antibody may, for example, be an IgG antibody, e.g. an IgG1 antibody.

In some embodiments, the monoclonal anti-Factor D antibody is an antibody fragment, e.g. a Fab fragment.

In some embodiments, the monoclonal anti-Factor D antibody is humanized.

In some embodiments, the anti-Factor D antibody present in the pre-lyophilized or lyophilized pharmaceutical formulations of the present invention is lampalizumab.

In some embodiments, the pre-lyophilized or lyophilized pharmaceutical formulation comprises about 25 mg/mL of lampalizumab.

In some embodiments, in the pre-lyophilized or lyophilized pharmaceutical formulation the lyoprotectant to antibody ratio is about 60 to 100 mole lyoprotectant:1 mole antibody.

In some embodiments, in the lyophilized formulation the lyoprotectant to antibody ratio is about 80 mole lyoprotectant:1 mole antibody.

In another aspect, the invention concerns a reconstituted aqueous liquid formulation prepared from a lyophilized pharmaceutical formulation hereinabove described or otherwise disclosed.

In some embodiments, the reconstituted formulation is for intraocular administration, such as, for example, for intravitreal administration.

In some embodiments, the reconstituted formulation is sterile.

In some embodiments, the reconstituted formulation comprises about 100 mg/mL of lampalizumab.

In a further aspect, the invention concerns a reconstituted aqueous liquid pharmaceutical formulation comprising a therapeutically effective amount of a monoclonal anti-Factor D antibody, about 20 mM to about 60 mM of histidine chloride, a polyol, sodium chloride and a surfactant.

In some embodiments, the anti-Factor D antibody present in the reconstituted aqueous liquid formulation comprises heavy chain hypervariable regions (HVR-HCs) having at least 98% or at least 99% sequence identity to the HVR sequences of HVR1-HC: GYTFTNYGMN (SEQ ID NO: 3); HVR2-HC: WINTYTGETTYADDFKG (SEQ ID NO: 4); HVR3-HC: EGGVNN (SEQ ID NO: 5) and/or light chain hypervariable regions (HVR-LCs) having at least 98% or at least 99% sequence identity to the HVR-LC sequences of HVR1-LC: ITSTDIDDDMN (SEQ ID NO: 8); HVR2-LC: GGNTLRP (SEQ ID NO: 9); and HVR3-LC: LQSDSLPYT (SEQ ID NO: 10).

In some embodiments, the reconstituted formulation comprises a monoclonal anti-Factor D antibody, which comprises the heavy chain hypervariable regions (HVR-HCs) of HVR1-HC: GYTFTNYGMN (SEQ ID NO: 3); HVR2-HC: WINTYTGETTYADDFKG (SEQ ID NO: 4); HVR3-HC: EGGVNN (SEQ ID NO: 5) and/or the light chain hypervariable regions (HVR-LCs) of HVR-LC sequences of HVR1-LC: ITSTDIDDDMN (SEQ ID NO: 8); HVR2-LC: GGNTLRP (SEQ ID NO: 9); and HVR3-LC: LQSDSLPYT (SEQ ID NO: 10).

In some embodiments, the monoclonal anti-Factor D antibody present in the reconstituted formulation comprises a heavy chain variable region sequence having at least 85%, or at least 90%, or at least 95%, or at least 98%, or at least 99% sequence identity to the heavy chain of SEQ ID NO: 2 and/or a light chain variable region sequence having at least 85%, or at least 90%, or at least 95%, or at least 98%, or at least 99% sequence identity to the light chain of SEQ ID NO: 7.

In some embodiments, the monoclonal anti-Factor D antibody present in the reconstituted formulation comprises a heavy chain sequence comprising SEQ ID NO: 2 and/or a light chain sequence comprising SEQ ID NO: 7.

In some embodiments, the monoclonal anti-Factor D antibody present in the reconstituted formulation is an IgG antibody, such as an IgG1 antibody.

In some embodiments, the monoclonal anti-Factor D antibody present in the reconstituted formulation is an antibody fragment, such as, for example, a Fab fragment.

In some embodiments, the monoclonal anti-Factor D antibody present in the reconstituted formulation is humanized.

In some embodiments, the anti-Factor D antibody present in the reconstituted formulation is lampalizumab.

In some embodiments, the reconstituted formulation is for intraocular, such as intravitreal administration.

In some embodiments, the reconstituted formulation is sterile.

In some embodiments, the reconstituted formulation comprises about 100 mg/mL lampalizumab.

In some embodiments, the reconstituted formulation has an ionic strength equivalent to about 37 to 88 mM sodium chloride, such as an ionic strength equivalent to about 63 mM sodium chloride.

In a further aspect, the invention concerns a lyophilized formulation comprising a monoclonal anti-Factor D antibody, wherein said lyophilized formulation upon reconstitution yields an aqueous liquid formulation comprising a therapeutically effective amount of said anti-Factor D antibody, about 20 mM to about 60 mM of histidine chloride, a polyol, sodium chloride and a surfactant.

In some embodiments, in the lyophilized formulation the polyol to antibody ratio is about 80 mole polyol:1 mole antibody.

In some embodiments, the anti-Factor D antibody present in the lyophilized formulation comprises heavy chain hypervariable regions (HVRs) having at least 98% or at least 99% sequence identity to the HVR sequences of HVR1-HC: GYTFTNYGMN (SEQ ID NO: 3); HVR2-HC: WINTYTGETTYADDFKG (SEQ ID NO: 4); HVR3-HC: EGGVNN (SEQ ID NO: 5) and/or light chain hypervariable regions (HVR-LCs) having at least 98% or at least 99% sequence identity to the HVR-LC sequences of HVR1-LC: ITSTDIDDDMN (SEQ ID NO: 8); HVR2-LC: GGNTLRP (SEQ ID NO: 9); and HVR3-LC: LQSDSLPYT (SEQ ID NO: 10).

In some embodiments, the anti-Factor D antibody present in the lyophilized formulation comprises the heavy chain hypervariable regions (HVR-HCs) of HVR1-HC: GYTFTNYGMN (SEQ ID NO: 3); HVR2-HC: WINTYTGETTYADDFKG (SEQ ID NO: 4); HVR3-HC: EGGVNN (SEQ ID NO: 5) and/or the light chain hypervariable regions (HVR-LCs) of HVR1-LC: ITSTDIDDDMN (SEQ ID NO: 8); HVR2-LC: GGNTLRP (SEQ ID NO: 9); and HVR3-LC: LQSDSLPYT (SEQ ID NO: 10).

In some embodiments, the monoclonal anti-Factor D antibody present in the lyophilized formulation comprises a heavy chain variable region sequence having at least 85%, or at least 90%, or at least 95%, or at least 98%, or at least 99% sequence identity to the heavy chain of SEQ ID NO: 2 and/or a light chain variable region sequence having at least 85%, or at least 90%, or at least 95%, or at least 98%, or at least 99% sequence identity to the light chain of SEQ ID NO: 7.

In some embodiments, the monoclonal anti-Factor D antibody present in the lyophilized formulation comprises a heavy chain sequence comprising SEQ ID NO: 2 and/or a light chain sequence comprising SEQ ID NO: 7.

In some embodiments, the monoclonal anti-Factor D antibody present in the lyophilized formulation is an IgG antibody, such as an IgG1 antibody.

In some embodiments, the monoclonal anti-Factor D antibody present in the lyophilized formulation is an antibody fragment, e.g. a Fab fragment.

In some embodiments, the monoclonal anti-Factor D antibody present in the lyophilized formulation is humanized.

In some embodiments, the anti-Factor D antibody present in the lyophilized formulation is lampalizumab.

In some embodiments, the aqueous liquid formulation yielded by reconstitution of the lyophilized formulation herein is for intravitreal administration.

In some embodiments, the lyophilized formulation is sterile.

In some embodiments, the lyophilized formulation comprises about 100 mg/mL lampalizumab.

In some embodiments, the lyophilized formulation is stable at a storage temperature of 5° C. for at least one year, or for at least two years.

In some embodiments, the aqueous liquid formulation yielded by reconstitution of the lyophilized formulation has an ionic strength equivalent to about 37 to 88 mM sodium chloride.

In some embodiments, the aqueous liquid formulation yielded by reconstitution of the lyophilized formulation has an ionic strength equivalent to about 63 mM sodium chloride.

In a further aspect, the invention concerns a syringe for intravitreal injection comprising any of the reconstituted formulations hereinabove described, or otherwise disclosed herein.

In another aspect, the invention concerns a method of making a pharmaceutical formulation comprising:

(a) preparing any of the previously described, or otherwise disclosed, formulations; and

(b) evaluating physical stability, chemical stability, or biological activity of the monoclonal anti-Factor D antibody in the formulation.

In yet another aspect, the invention concerns a method for treatment of a complement-associated ocular disease comprising administering to a subject in need any of the foregoing reconstituted formulations.

In some embodiments, the complement-associated ocular disease is selected from the group consisting of age-related macular degeneration (AMD), diabetic retinopathy, choroidal neovascularization (CNV), uveitis, diabetic macular edema, pathological myopia, von Hippel-Lindau disease, histoplasmosis of the eye, Central Retinal Vein Occlusion (CRVO), corneal neovascularization, and retinal neovascularization.

In some embodiments, the AMD is dry AMD.

In some embodiments, the dry AMD is characterized by geographic atrophy.

In some embodiments, the formulation is administered by intravitreal injection.

In a different aspect, the invention concerns use of any of the reconstituted formulations herein for treatment of a complement-associated ocular disease.

In some embodiments, the complement-associated ocular disease is selected from the group consisting of age-related macular degeneration (AMD), diabetic retinopathy, choroidal neovascularization (CNV), uveitis, diabetic macular edema, pathological myopia, von Hippel-Lindau disease, histoplasmosis of the eye, Central Retinal Vein Occlusion (CRVO), corneal neovascularization, and retinal neovascularization.

In some embodiments, the AMD is dry AMD.

In some embodiments, the dry AMD is characterized by geographic atrophy.

In some embodiments, the formulation is for intravitreal administration.

In all embodiments, the formulations herein, including pre-lyophilized, lyophilized. reconstituted formulations, and liquid formulations, may comprise anti-Factor D antibody variants.

In some embodiments, the monoclonal anti-Factor D antibody present in the formulations of this invention comprises heavy chain hypervariable regions (HVR-HCs) having at least 90%, or at least 95%, or at least 98%, or at least 99% sequence identity to the heavy and/or light chain CDR sequences of anti-Factor D antibody variants AFD.v1-AFD.v15 (see FIG. 20).

In some embodiments, the monoclonal anti-Factor D antibody comprises the heavy and/or light chain CDR sequence of anti-Factor D antibody variants AFD.v1-AFD.v15 (see FIG. 20).

In some embodiments, the monoclonal anti-Factor D antibody comprises a heavy chain variable region sequence having at least 85%, or at least 90%, or at least 95%, or at least 98%, or at least 99% sequence identity to the variable region sequence of the light chain and/or heavy chain of anti-Factor D antibody variants AFD.v1-AFD.v15 (see FIGS. 21 and 22).

In some embodiments, the monoclonal anti-Factor D antibody comprises the light chain and/or heavy chain variable region sequence of anti-Factor D antibody variants AFD.v1-AFD.v15 (see FIGS. 21 and 22).

In some embodiments, the C-terminus of the heavy chain of the Fab fragment ends in the sequence CDKTHX (SEQ ID NO: 52), wherein X is any amino acid except T. The present invention specifically includes formulations comprising anti-Factor D antibodies as hereinabove described and anti-Factor D antibody variants (e.g. AFD.v1-AFD.v15) with the C-terminal terminus of the heavy chain of a Fab fragment ending in the amino acids “CDKTHT” (SEQ ID NO: 11), “CDKTHL” (SEQ ID NO: 12), “CDKTH” (SEQ ID NO: 13), “CDKT” (SEQ ID NO: 14), “CDK” (SEQ ID NO: 15), or “CD”. Truncations of the C terminus are able to eliminate AHA-reactivity against the Fab, without compromising thermostability or expression. In some embodiments, the C-terminus of the heavy chain of a Fab fragment of an anti-Factor D antibody or antibody variant (e.g. AFD.v1-AFD.v15) ends in the amino acids “CDKTHTC” (SEQ ID NO: 16), “CDKTHTCPPC” (SEQ ID NO: 17), “CDKTHTCPPS” (SEQ ID NO: 18), “CDKTHTSPPC” (SEQ ID NO: 19), “CDKTHTAPPC” (SEQ ID NO: 20), “CDKTHTSGGC” (SEQ ID NO: 21), or “CYGPPC” (SEQ ID NO: 22). In some such embodiments, a free cysteine in the C-terminal amino acids may be amenable to conjugation, for example, to a polymer such as PEG. In some embodiments, a Fab fragment comprises a heavy chain constant region selected from SEQ ID NOs: 30 to 51. In some embodiments, a Fab is an IgG2 or IgG4 Fab (See, e.g. SEQ ID NOs: 43 to 50) (FIG. 19). In some embodiments, a Fab is an IgG2 Fab fragment comprising a heavy chain constant region of SEQ ID NO: 43 (VERK; SEQ ID NO: 23) or IgG2 Fab-C fragment comprising a heavy chain constant region of SEQ ID NO: 44 (VERKC; SEQ ID NO: 24). In some embodiments, a Fab is an IgG4 fragment comprising a heavy chain constant region selected from SEQ ID NO: 46 (KYGPP; SEQ ID NO: 26), SEQ ID NO: 50 (KYGP; SEQ ID NO: 27), SEQ ID NO: 47 (KYG, SEQ ID NO: 28), SEQ ID NO: 48 (KY), and SEQ ID NO: 49 (K) or an IgG4 Fab-C fragment comprising a heavy chain constant region of SEQ ID NO: 45 (KYGPPC; SEQ ID NO: 25).

As an alternative to truncating and/or mutation at the C terminus, to avoid pre-existing anti-hinge antibody (PE-AHA) responses, IgG2 or IgG4 Fab fragments can be used, since these do not show PE-AHA response.

In some embodiments, the anti-Factor D antibody variant present in the formulations of the present invention AFD.v8 or AFD.v14.

### BRIEF DESCRIPTION OF THE DRAWINGS

FIG. 1 illustrates the role of Factor D in the alternative complement pathway.

FIG. 2 shows the dependence of lampalizumab solubility on basic charge variant levels. Each dialysis contains lampalizumab at 115 mg/mL in 30 mM HisCl and 12 mM NaCl at pH 5.6 at ambient temperature. Both cassettes contain lampalizumab from the same lot but the sample in the cassette on the right (B) was titrated to pH 5.5 and stressed until it contained 27% basic peak by IEC. The starting material contained 7% basic peak by IEC (A).

FIG. 3 illustrates lampalizumab solubility as a function of NaCl concentration and basic charge variant levels. Each vial contains lampalizumab at 115 mg/mL in 30 mM HisCl at pH 5.6 at ambient lab temperature. The NaCl concentration in each vial in mM (0, 6, 12, 14, 16, 18, 20, 22, 24) is shown. 12 mM of NaCl is required to ensure complete solubility (clear solution with no turbidity) of lampalizumab initially (A), but 24 mM of NaCl is required to ensure complete solubility (clear solution with no turbidity) of lampalizumab when higher levels of basic charge variants are present (B).

FIG. 4 shows Drug Substance size variants by SEC as a function of time at 30° C.

FIG. 5 shows Drug Substance charge variants by IEC as a function of time at 30° C.

FIG. 6 shows Drug Substance size variants by SEC as a function of time at −20° C.

FIG. 7 shows Drug Substance charge variants by IEC as a function of time at −20° C.

FIG. 8 shows Drug Product size variants by SEC as a function of time at 40° C./75% RH.

FIG. 9 shows Drug Product aggregation rate by SEC 40° C./75% RH as a function of the sugar-to-protein ratio in the formulation.

FIG. 10 is an overlay of Drug Product Formulation #1 SEC chromatograms after storage at 40° C./75% RH for 0, 2, and 4 weeks.

FIG. 11 shows Drug Product charge variants by IEC as a function of time at 40° C./75% RH.

FIG. 12 shows Drug Product size variants by SEC as a function of time at 25° C./60% RH.

FIG. 13 shows Drug Product size variants by SEC as a function of time at 5° C.

FIG. 14 shows Drug Product charge variants by IEC as a function of time at 5° C.

FIG. 15 shows The nucleotide sequence of the heavy chain of lampalizumab (humanized anti-Factor D Fab 238-1) (SEQ ID NO: 1). The nucleotide sequence encodes for the heavy chain of lampalizumab with the start and stop codon shown in bold and underlined. The codon corresponding to the first amino acid in FIG. 18 is bold and italicized.

FIG. 16 shows the amino acid sequence of the heavy chain of lampalizumab (humanized anti-Factor D Fab 238-1) (SEQ ID NO: 2). The HVR-HC sequences are bold and italicized. Variable regions are regions not underlined while first constant domain CH1 is underlined. HVR-HC regions are shown as: HVR1-HC: GYTFTNYGMN (SEQ ID NO: 3); HVR2-HC: WINTYTGETTYADDFKG (SEQ ID NO: 4); HVR3-HC: EGGVNN (SEQ ID NO: 5). FIG. 16 also discloses FR1-FR4 and CH1 sequences as SEQ ID NOS 54-57 and 30, respectively.

FIG. 17 shows the nucleotide sequence of the light chain of lampalizumab (humanized anti-Factor D Fab 238-1) (SEQ ID NO: 6). The nucleotide sequence encodes for the light chain of lampalizumab with the start and stop codon shown in bold and underlined. The codon corresponding to the first amino acid in FIG. 20 is bold and italicized.

FIG. 18 shows the amino acid sequence of the light chain of lampalizumab (humanized anti-Factor D Fab 238-1) (SEQ ID NO: 7). The amino acid sequence lacks the N-terminal signal sequence. The HVR-LC sequences are bold and italicized. Variable regions are regions not underlined while first constant domain CL1 is underlined. Framework (FR) regions and HVR regions are shown as: HVR1-LC: ITSTDIDDDMN (SEQ ID NO: 8); HVR2-LC: GGNTLRP (SEQ ID NO: 9); HVR3-LC: LQSDSLPYT (SEQ ID NO: 10). FIG. 18 also discloses FR1-FR4 and CH1 sequences as SEQ ID NOS 58-61 and 29, respectively.

FIG. 19 shows the Fab light chain constant region sequence of an IgG1 anti-Factor D antibody Fab fragment (SEQ ID NO: 29), and the heavy chain constant region sequences of IgG1, IgG2 and IgG4 anti-Factor D antibodies, including heavy chains with C-terminal truncations (SEQ ID NOs: 30-51).

FIG. 20 shows the light and heavy chain CDR sequences of anti-Factor D antibody variants AFD.v1-AFD.v15. CDR L1 sequences disclosed as SEQ ID NOS 8, 62-68, 68-70, 69, 69, 69, 69, 69 and 69, respectively, in order of appearance. CDR L2 sequences “GGNTLRP” and “AASTLQS” disclosed as SEQ ID NOS 9 and 71, respectively. CDR L3 sequences “LQSDSLPYT,” “QKYNSAPYT” and “LQSESLPYT” disclosed as SEQ ID NOS 10, 72 and 73, respectively. CDR H1 sequences “NYGMN” and “SYAMN” disclosed as SEQ ID NOS 74 and 75, respectively. CDR H2 sequences “WINTYTGETTYADDFKG,” “WINTNTGNPTYAQGFTG,” “WINTYTGETTYAEDFKG” and “WISTYTGETTYAEDFKG” disclosed as SEQ ID NOS 4, 76, 77 and 78, respectively. CDR H3 sequences “EGGVNN,” “EGYFDY,” “EGGVDN,” “EGGVQN” and “EGGVNN” disclosed as SEQ ID NOS 5, 79, 80, 81 and 82, respectively.

FIG. 21 shows the alignment of the light chain variable region sequences of anti-Factor D antibody variants AFD.v1-AFD.v15 in alignment with human framework and lampalizumab light chain variable region sequences (SEQ ID NOS 83-94, 92, 92, 92, 92 and 94, respectively, in order of appearance). The CDR sequences according to Kabat definition are underlined.

FIG. 22 shows the alignment of the heavy chain variable region sequences of anti-Factor D antibody variants AFD.va-AFD.v15 in alignment with human framework and lampalizumab heavy chain variable region sequence (SEQ ID NOS 95, 96, 95, 95, 95, 95, 95, 97, 97, 97, 97, 97-101 and 101, respectively, in order of appearance). The CDR sequences according to Kabat definition are underlined.

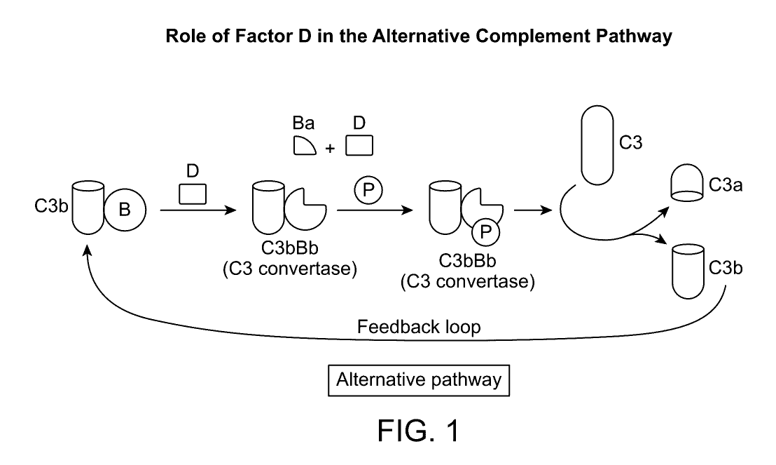

Table 1. Drug Substance Formulations Screened.

Table 2. Stability data for Drug Substance formulations stored at −20° C.

Table 3. Stability data for Drug Substance formulations stored at 5° C.

Table 4. Stability data for Drug Substance formulations stored at 30° C.

Tables 5A and 5B. Stability data for Drug Product formulations stored at 5° C.

Tables 6A and 6B. Stability data for Drug Product formulations stored at 25° C./65% RH.

Tables 7A and 7B. Stability data for Drug Product formulations stored at 40° C./75% RH.

Table 8. ELISA binding data for formulations 1 and 7 at select time points.

Table 9. Stability data for Phase III lampalizumab Drug Substance.

Tables 10A and 10B. Stability data for Phase III lampalizumab Drug Product.

### DETAILED DESCRIPTION

### I. Definitions

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges encompassed within the invention, subject to any specifically excluded limit in the stated range.

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), provides one skilled in the art with a general guide to many of the terms used in the present application.

All publications mentioned herein are expressly incorporated herein by reference to disclose and describe the methods and/or materials in connection with which the publications are cited.

The term “antibody” is used in the broadest sense, and specifically covers full length monoclonal antibodies, polyclonal antibodies, multispecific antibodies (e.g., bispecific antibodies) and antibody fragments so long as they exhibit the desired biological activity such as antigen-binding activity. Antibodies (Abs) and immunoglobulins (Igs) are glycoproteins having the same structural characteristics. While antibodies exhibit binding specificity to a specific target, immunoglobulins include both antibodies and other antibody-like molecules which lack target specificity. Native antibodies and immunoglobulins are usually heterotetrameric glycoproteins of about 150,000 daltons, composed of two identical light (L) chains and two identical heavy (H) chains. Each heavy chain has at one end a variable domain (V_{H}) followed by a number of constant domains. Each light chain has a variable domain at one end (V_{L}) and a constant domain at its other end. The term “Antibody” as used herein expressly encompasses antibody fragments retaining antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, Fab′-C, Fab-SH, Fab-C, Fab-C-SH, Fab′-C-SH F(ab′)2; diabodies; linear antibodies; single-chain antibody molecules (e.g., scFv); and multispecific antibodies formed from antibody fragments.

A “Fab-C” refers to a Fab with a C-terminal cysteine, which may be a native cysteine that occurs at that residue position (such as a cysteine from the hinge region), or may be a cysteine added to the C-terminus that does not correspond to a native cysteine. Nonlimiting exemplary Fab-C heavy chain constant regions include the sequences of SEQ ID NOs: 32, 44 and 45.

A “Fab-SH” refers to a Fab with a free thiol group. In some embodiments, the free thiol group is located in the last 10 amino acids of the C-terminus of the Fab. Fab-C antibodies are typically also Fab-SH antibodies. A further nonlimiting exemplary Fab-SH heavy chain constant region having the amino acid sequence of SEQ ID NO: 34. Typically, a Fab comprising an engineered cysteine (i.e., a Fab that is a THIOMAB) is a Fab-SH.

As used herein, an “anti-Factor D antibody” means an antibody, as hereinabove defined, which specifically binds to Factor D in such a manner so as to inhibit or substantially reduce complement activation. In some embodiments, the anti-Factor D antibody is an antibody fragment (as hereinabove defined), such as a Fab fragment.

The term “Factor D” is used herein to refer to native sequence and variant Factor D polypeptides. In some embodiments the term “Factor D” refers to a native sequence mammalian polypeptide, more preferably a native sequence human polypeptide.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6th ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-binding sites and is still capable of cross-linking antigen.

The Fab fragment also contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region. Fab′-SH is the designation herein for Fab′ in which the cysteine residue(s) of the constant domains bear at least one free thiol group. F(ab′)2 antibody fragments originally were produced as pairs of Fab′ fragments which have hinge cysteines between them. Other chemical couplings of antibody fragments are also known.

As used herein, a “Fab” refers to an antibody that comprises a heavy chain constant region that comprises the CH1 domain, or a sufficient portion of the CH1 domain to form a disulfide bond with the light chain constant region, but does not contain a CH2 domain or a CH3 domain. As used herein, a Fab may comprise one or more amino acids of the hinge region. Thus, as used herein, the term “Fab” encompasses Fab′ antibodies. A Fab may comprise additional non-native amino acids, such as a C-terminal cysteine, in which case it may be referred to as a Fab-C. As discussed below, the term Fab-C also encompasses Fabs comprising native amino acids of the hinge region, including a native cysteine at the C-terminus. In some embodiments, a Fab comprises an engineered cysteine (i.e., a Fab may be a THIOMAB).

“Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the VH-VL dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The term “hypervariable region” or “HVR,” as used herein, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the “complementarity determining regions” (CDRs), the latter being of highest sequence variability and/or involved in antigen recognition. HVR-H3 is believed to play a unique role in conferring fine specificity to antibodies. See, e.g., Xu et al. (2000) Immunity 13:37-45; Johnson and Wu (2003) in Methods in Molecular Biology 248:1-25 (Lo, ed., Human Press, Totowa, N.J.). “Framework Region” or “FR” residues are those variable domain residues other than the hypervariable region residues as herein defined. An HVR region as used herein comprise any number of residues located within positions 24-36 (for L1), 46-56 (for L2), 89-97 (for L3), 26-35B (for H1), 47-65 (for H2), and 93-102 (for H3). Therefore, an HVR includes residues in positions described previously:
A) 24-34 (L1), 50-52 (L2), 91-96 (L3), 26-32 (H1), 53-55 (H2), and 96-101 (H3) (Chothia and Lesk, J. Mol. Biol. 196:901-917 (1987);
B) 24-34 of L1, 50-56 of L2, 89-97 of L3, 31-35B of H1, 50-65 of H2, and 95-102 of H3 (Kabat et al., Sequences of Proteins of Immunological Interest, 5th Ed. Public Health Service, National Institutes of Health, Bethesda, Md. (1991).
C) 30-36 (L1), 46-55 (L2), 89-96 (L3), 30-35 (H1), 47-58 (H2), 93-100a-j (H3) (MacCallum et al. J. Mol. Biol. 262:732-745 (1996).

Hypervariable regions may comprise “extended hypervariable regions” as follows: 24-36 or 24-34 (L1), 46-56 or 50-56 (L2) and 89-97 (L3) in the VL and 26-35B (H1), 50-65, 47-65 or 49-65 (H2) and 93-102, 94-102 or 95-102 (H3) in the VH. The variable domain residues are numbered according to Kabat et al., supra for each of these definitions.

With the exception of CDR1 in VH, CDRs generally comprise the amino acid residues that form the hypervariable loops. CDRs also comprise “specificity determining residues,” or “SDRs,” which are residues that contact antigen. SDRs are contained within regions of the CDRs called abbreviated-CDRs, or a-CDRs. Exemplary a-CDRs (a-CDR-L1, a-CDR-L2, a-CDR-L3, a-CDR-H1, a-CDR-H2, and a-CDR-H3) occur at amino acid residues 31-34 of L1, 50-55 of L2, 89-96 of L3, 31-35B of H1, 50-58 of H2, and 95-102 of H3. (See Almagro and Fransson, Front. Biosci. 13:1619-1633 (2008).)

An “antibody variant” or “modified antibody” of a reference antibody (also referred to as “starting antibody” or “parent antibody”) is an antibody that comprises an amino acid sequence different from that of the reference/starting antibody, wherein one or more of the amino acid residues of the reference antibody have been modified. Generally, an antibody variant will possess at least 80% sequence identity, preferably at least 90% sequence identity, more preferably at least 95% sequence identity, and most preferably at least 98% sequence identity with the reference antibody. Percentage sequence identity is determined for example, by the Fitch et al., Proc. Natl. Acad. Sci. USA, 80: 1382-1386 (1983), version of the algorithm described by Needleman et al., J. Mol. Biol., 48: 443-453 (1970), after aligning the sequences of the reference antibody and the candidate antibody variant to provide for maximum homology. Identity or similarity is defined herein as the percentage of amino acid residues in the candidate variant sequence that are identical (i.e. same residue) or similar (i.e. amino acid residue from the same group based on common side-chain properties, see below) with the parent antibody residues, after aligning the sequences and introducing gaps, if necessary, to achieve the maximum percent sequence identity. Amino acid sequence variants of an antibody may be prepared by introducing appropriate nucleotide changes into DNA encoding the antibody, or by peptide synthesis. Such variants include, for example, deletions from, and/or insertions into and/or substitutions of, residues within the amino acid sequence of the antibody of interest. Any combination of deletion, insertion, and substitution is made to arrive at the final construct, provided that the final construct possesses the desired characteristics. The amino acid changes also may alter post-translational processes of the antibody, such as changing the number or position of glycosylation sites. Methods for generating antibody sequence variants of antibodies are similar to those for generating amino acid sequence variants of polypeptides described in U.S. Pat. No. 5,534,615, expressly incorporated herein by reference, for example.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256:495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352:624-628 and Marks et al. (1991) J. Mol. Biol. 222:581-597, for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al. (1984) Proc. Natl. Acad. Sci. USA 81:6851-6855).

“Humanized” forms of non-human (e.g., murine) antibodies are chimeric antibodies which contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al. (1986) Nature 321:522-525; Riechmann et al. (1988) Nature 332:323-329; and Presta (1992) Curr. Op. Struct. Biol. 2:593-596.

A protein including an antibody is said to be “stable” if it essentially retains the intact conformational structure and biological activity. Various analytical techniques for measuring protein stability are available in the art and are reviewed in, e.g., Peptide and Protein Drug Delivery, 247-301, Vincent Lee Ed., Marcel Dekker, Inc., New York, N.Y., Pubs. (1991) and Jones (1993) Adv. Drug Delivery Rev. 10: 29-90. An antibody variant with “improved stability” refers to an antibody variant that is more stable comparing to the starting reference antibody. Preferably, antibody variants with improved stability are variants of the native (wild-type) antibodies in which specific amino acid residues are altered for the purpose of improving physical stability, and/or chemical stability, and/or biological activity, and/or reducing immunogenicity of the native antibodies. Walsh (2000) Nat. Biotech. 18:831-3.

The term “isomerization” refers generally to a chemical process by which a chemical compound is transformed into any of its isomeric forms, i.e., forms with the same chemical composition but with different structure or configuration and, hence, generally with different physical and chemical properties. Specifically used herein is aspartate isomerization, a process wherein one or more aspartic acid (D or Asp) residue(s) of a polypeptide have been transformed to isoaspartic acid (IsoAsp) and/or cyclic imide (Asu) residue(s). Geiger and Clarke (1987) J. Biol. Chem. 262:785-94; Wakankar et al. (2007) Biochem. 46:1534-44.

The term “deamidation” refers generally to a chemical reaction wherein an amide functional group is removed from an organic compound. Specifically used herein is asparagine deamidation, a process wherein one or more asparagine (N or Asn) residue(s) of a polypeptide have been converted to aspartic acid (D or Asp), i.e. the neutral amide side chain has been converted to a residue with an overall acidic property. Xie and Schowen (1999) J. Pharm. Sci. 88:8-13.

Amino acid residues “prone” to certain identified physical or chemical processes (e.g., isomerization or deamidation) refer to those residues within a specific protein molecule that have been identified to have the propensity to undergo the identified processes such as isomerization or deamidation. Their propensities are often determined by their relative positions within the primary and/or conformational structure of the protein. For example, it has been shown that the first Asp in an Asp-XXX motif (wherein XXX can be Asp, Gly, His, Ser or Thr) is prone to Asp isomerization due to the involvement of its adjacent residue, where some other Asp within the same protein may not possess such propensity. Assays for identifying residues to certain process within a specific protein molecule are known in the art. See, e.g., Cacia et al (1996) Biochem. 35:1897-1903.

“Active” or “activity” or “biological activity” in the context of an anti-factor D antibody of the present invention is the ability to antagonize (partially or fully inhibit) a biological activity of Factor D. One example of a biological activity of a Factor D antagonist is the ability to achieve a measurable improvement in the state, e.g. pathology, of a Factor D-associated disease or condition, such as, for example, a complement-associated ocular condition. The activity can be determined in in vitro or in vivo tests, including binding assays, alternative pathway hemolysis assays (e.g. assays measuring inhibition of the alternative pathway complement activity or activation), using a relevant animal model, or human clinical trials.

The term “complement-associated disorder” is used in the broadest sense and includes disorders associated with excessive or uncontrolled complement activation. They include complement activation during cardiopulmonary bypass operations; complement activation due to ischemia-reperfusion following acute myocardial infarction, aneurysm, stroke, hemorrhagic shock, crush injury, multiple organ failure, hypobolemic shock, intestinal ischemia or other events causing ischemia. Complement activation has also been shown to be associated with inflammatory conditions such as severe burns, endotoxemia, septic shock, adult respiratory distress syndrome, hemodialysis; anaphylactic shock, severe asthma, angioedema, Crohn's disease, sickle cell anemia, poststreptococcal glomerulonephritis and pancreatitis. The disorder may be the result of an adverse drug reaction, drug allergy, IL-2 induced vascular leakage syndrome or radiographic contrast media allergy. It also includes autoimmune disease such as systemic lupus erythematosus, myasthenia gravis, rheumatoid arthritis, Alzheimer's disease and multiple sclerosis. Complement activation is also associated with transplant rejection. Complement activation is also associated with ocular diseases such as age-related macular degeneration, diabetic retinopathy and other ischemia-related retinopathies, choroidal neovascularization (CNV), uveitis, diabetic macular edema, pathological myopia, von Hippel-Lindau disease, histoplasmosis of the eye, Central Retinal Vein Occlusion (CRVO), corneal neovascularization, and retinal neovascularization.

The term “complement-associated eye condition” or “complement-associated ocular condition” is used in the broadest sense and includes all eye conditions the pathology of which involves complement, including the classical and the alternative pathways, and in particular the alternative pathway of complement. Complement-associated eye conditions include, without limitation, macular degenerative diseases, such as all stages of age-related macular degeneration (AMD), including dry and wet (non-exudative and exudative) forms, choroidal neovascularization (CNV), uveitis, diabetic and other ischemia-related retinopathies, and other intraocular neovascular diseases, such as diabetic macular edema, pathological myopia, von Hippel-Lindau disease, histoplasmosis of the eye, Central Retinal Vein Occlusion (CRVO), corneal neovascularization, and retinal neovascularization. In one example, complement-associated eye conditions includes age-related macular degeneration (AMD), including non-exudative (e.g. intermediate dry AMD or geographic atrophy (GA)) and exudative (e.g. wet AMD (choroidal neovascularization (CNV)) AMD, diabetic retinopathy (DR), endophthalmitis and uveitis. In a further example, nonexudative AMD may include the presence of hard drusen, soft drusen, geographic atrophy and/or pigment clumping. In one example, complement-associated eye conditions include age-related macular degeneration (AMD), including early AMD (e.g. includes multiple small to one or more non-extensive medium sized drusen), intermediate AMD (e.g. includes extensive medium drusen to one or more large drusen) and advanced AMD (e.g. includes geographic atrophy or advanced wet AMD (CNV). (Ferris et al., AREDS Report No. 18; Sallo et al., Eye Res., 34(3): 238-40 (2009); Jager et al., New Engl. J. Med., 359(1): 1735 (2008)). In a further example, intermediate dry AMD may include large confluent drusen. In a further example, geographic atrophy may include photoreceptor and/or Retinal Pigmented Epithelial (RPE) loss. In a further example, the area of geographic atrophy may be small or large and/or may be in the macula area or in the peripheral retina. In one example, complement-associated eye condition is intermediate dry AMD. In one example, complement-associated eye condition is geographic atrophy. In one example, complement-associated eye condition is wet AMD (choroidal neovascularization (CNV)).

“Geographic Atrophy”, also referred to herein as “GA”, as used herein is a disease involving degeneration of the retinal pigment epithelium (RPE), associated with loss of photoreceptors. GA is the advanced form of dry AMD.

“GA Area”, as used herein refers to a discrete area representing loss of retinal anatomy (e.g. photoreceptors and retinal pigment epithelium (RPE). GA area is measured by standard imaging techniques such as fundus autofluorescence (FAF) and digital color fundus photography (CFP).

“Early AMD”, as used herein is a disease characterized by multiple small (<63 μ [symbol] ) or >1 intermediate drusen (>63 μ [symbol] and <125 μ [symbol] ).

“Intermediate AMD”, as used herein is a disease characterized by many intermediate or >1 large drusen (>125 μ [symbol] ) often accompanied by hyper or hypopigmentation of the retinal pigment epithelium.

“Advanced AMD”, as used herein is a disease characterized by geographic atrophy (GA) or neovascular (wet) AMD).

“Treatment” is an intervention performed with the intention of preventing the development or altering the pathology of a disorder. Accordingly, “treatment” refers to both therapeutic treatment and prophylactic or preventative measures. Those in need of treatment include those already with the disorder as well as those in which the disorder is to be prevented. In treatment of an immune related disease, a therapeutic agent may directly alter the magnitude of response of a component of the immune response, or render the disease more susceptible to treatment by other therapeutic agents, e.g., antibiotics, antifungals, anti-inflammatory agents, chemotherapeutics, etc.

The “pathology” of a disease, such as a complement-associated disorder, includes all phenomena that compromise the well-being of the patient. This includes, without limitation, abnormal or uncontrollable cell growth (neutrophilic, eosinophilic, monocytic, lymphocytic cells), antibody production, auto-antibody production, complement production, interference with the normal functioning of neighboring cells, release of cytokines or other secretory products at abnormal levels, suppression or aggravation of any inflammatory or immunological response, infiltration of inflammatory cells (neutrophilic, eosinophilic, monocytic, lymphocytic) into cellular spaces, etc.

The term “mammal” as used herein refers to any animal classified as a mammal, including, without limitation, humans, higher primates, domestic and farm animals, and zoo, sports or pet animals such horses, pigs, cattle, dogs, cats and ferrets, etc. In some embodiments of the invention, the mammal is a human.

Administration “in combination with” one or more further therapeutic agents includes simultaneous (concurrent) and consecutive administration in any order.

“Therapeutically effective amount” is the amount of a “Factor D antibody” which is required to achieve a measurable improvement in the state, e.g. pathology, of the target disease or condition, such as, for example, a complement-associated eye condition.

“Pharmaceutically acceptable” excipients (vehicles, additives) are those which can reasonably be administered to a subject mammal to provide an effective dose of the active ingredient employed.

A “stable” formulation in one in which the protein, e.g. an anti-Factor D antibody, therein essentially retains its physical stability and/or chemical stability and/or biological activity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs (1991) and Jones, A. Adv. Drug Delivery Rev. 10: 29-90 (1993), for example. Stability can be measured at a selected temperature for a selected time period. In one embodiment, the formulation is stable at room temperature or at 40° C. for at least 1 month and/or stable at 2-8° C. for at least 1 year and preferably for at least 2 years. In another embodiment, the pre-lyophilized formulation (also referred herein as “Drug Substance” or “DS”) is stable at a storage temperature of −20° C. for at least one year, or for at least two years, or for at least three years, or for at least five years. In a further embodiment, the lyophilized formulation is stable at a storage temperature of 5° C. for at least one year, or for at least two years, or for at least three years, or for at least four years, or for at least five years. Furthermore, the formulation is preferably stable following freezing (to, e.g., −70° C.) and thawing of the formulation.

A protein, such as an anti-Factor D antibody, “retains its physically stability” in a pharmaceutical formulation if it shows no signs of aggregation, precipitation and/or denaturation upon visual examination of color and/or clarity, or as measured by UV light scattering or by size exclusions chromatography.

A protein, e.g. an anti-Factor D antibody, “retains the chemical stability” in a pharmaceutical formulation, if the chemical stability at a given time is such that the protein is considered to still retain its biological activity as defined below. Chemical stability can be assessed by detecting and quantifying chemically altered forms of the protein. Chemical alteration may involve size modification (e.g. clipping) which can be evaluated using size exclusion chromatography, SDS-PAGE and/or matrix-assisted laser desorption ionization/time-of-flight mass spectrometry (MALDI/TOF MS), for example. Other types of chemical alteration include charge alteration (e.g. occurring as a result of deamidation) which can be evaluated by ion-exchange chromatography, for example.

An antibody, e.g. an anti-Factor D antibody, “retains its biological activity” in a pharmaceutical formulation, if the biological activity of the antibody at a given time is within about 10% (within the errors of the assay) of the biological activity exhibited at the time the pharmaceutical formulation was prepared as determined in an antigen binding assay, for example. Other “biological activity” assays for antibodies are elaborated herein below.

By “isotonic” is meant that the formulation of interest has essentially the same osmotic pressure as human blood. Isotonic formulations will generally have an osmotic pressure from about 250 to 350 mOsm/kg. Isotonicity can be measured using a vapor pressure or ice-freezing type osmometer for example.

The term “lyoprotectant” refers to a substance, such as a chemical compound or molecule, that protects a protein, e.g. an antibody, from damage resulting from lyophilization. Preferably, the lyoprotectant is a polyol.

A “polyol” is a substance with multiple hydroxyl groups, and includes sugars (reducing and nonreducing sugars), sugar alcohols and sugar acids. Preferred polyols herein have a molecular weight which is less than about 600 D (e.g. in the range from about 120 to about 400 D). A “reducing sugar” is one which contains a hemiacetal group that can reduce metal ions or react covalently with lysine and other amino groups in proteins and a “nonreducing sugar” is one which does not have these properties of a reducing sugar. Examples of reducing sugars are fructose, mannose, maltose, lactose, arabinose, xylose, ribose, rhamnose, galactose and glucose. Nonreducing sugars include sucrose, trehalose, sorbose, melezitose and raffinose Mannitol, xylitol, erythritol, threitol, sorbitol and glycerol are examples of sugar alcohols. As to sugar acids, these include L-gluconate and metallic salts thereof. Where it desired that the formulation is freeze-thaw stable, the polyol is preferably one which does not crystallize at freezing temperatures (e.g. −20° C.) such that it destabilizes the antibody in the formulation. Polyols, including mixtures of polyols, can be used as lyoprotectants in the formulations of the present invention. Nonreducing sugars such as sucrose and trehalose are preferred as lyoprotectants in the anti-Factor D antibody formulations herein, sucrose is being preferred over trehalose.

As used herein, “buffer” refers to a buffered solution that resists changes in pH by the action of its acid-base conjugate components. The buffer of this invention has a pH in the range from 5.0 to 5.4; and most preferably has a pH of about 5.3. Examples of buffers that will control the pH in this range include histidine, acetate (e.g. sodium acetate), succinate (such as sodium succinate), gluconate, citrate and other organic acid buffers. Where a freeze-thaw stable formation is desired, the buffer is preferably not phosphate. The term “buffer” specifically includes combinations of two or more buffers suitable for providing the desired pH in the formulations herein.

As used herein, a “surfactant” refers to a surface-active agent, typically a nonionic surfactant. The formulations of the present invention comprise one or more surfactant. Thus, the term “surfactant” specifically includes mixtures of two or more surfactants. Examples of suitable surfactants include polysorbate (for example, polysorbate 20 and polysorbate 80); poloxamer (e.g. poloxamer 188); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g. lauroamidopropyl); myristamidopropyl-, palmidopropyl-, or isostearamidopropyl-dimethylamine; sodium methyl cocoyl-, or disodium methyl oleyl-taurate; polyethyl glycol, polypropyl glycol, and copolymers of ethylene and propylene glycol (e.g. Pluronics, PF68 etc). In some embodiments, the surfactant herein is a polysorbate, e.g. polysorbate 20 or a poloxamer.

A “preservative” is a compound which can be included in the formulation to essentially reduce bacterial action therein, thus facilitating the production of a multi-use formulation, for example. Examples of potential preservatives include octadecyldimethylbenzyl ammonium chloride, hexamethonium chloride, benzalkonium chloride (a mixture of alkylbenzyldimethylammonium chlorides in which the alkyl groups are long-chain compounds), and benzelthonium chloride. Other types of preservatives include aromatic alcohols such as phenol, butyl and benzyl alcohol, alkyl parabens such as methyl or propyl paraben, catechol, resorcinol, cyclohexanol, 3-pentanol, and m-cresol. The term “preservative” specifically includes mixtures of two or more preservatives. The most preferred preservative herein is benzyl alcohol.

The terms “long-acting delivery”, “sustained-release” and “controlled release” are used generally to describe a delivery mechanism using formulation, dosage form, device or other types of technologies to achieve the prolonged or extended release or bioavailability of a therapeutic drug. It may refer to technologies that provide prolonged or extended release or bioavailability of the drug to the general systemic circulation or a subject or to local sites of action in a subject including (but not limited to) cells, tissues, organs, joints, regions, and the like. Furthermore, these terms may refer to a technology that is used to prolong or extend the release of the drug from a formulation or dosage form or they may refer to a technology used to extend or prolong the bioavailability or the pharmacokinetics or the duration of action of the drug to a subject or they may refer to a technology that is used to extend or prolong the pharmacodynamic effect elicited by a formulation. A “long-acting formulation,” a “sustained release formulation,” or a “controlled release formulation” is a pharmaceutical formulation, dosage form, or other technology that is used to provide long-acting delivery. In one aspect, the controlled release is used to improve drug's local bioavailability, specifically ocular residence time in the context of ocular delivery. “Increased ocular residence time” refers to the post-delivery period during which the delivered ocular drug remains effective both in terms of quality (stay active) and in terms of quantity (effective amount). In addition to or in lieu of high dose and controlled release, the drug can be modified post-translationally, such as via PEGylation, to achieve increased in vivo half-life.

### II. Detailed Description

Anti-Factor D Antibody Formulations

The invention herein pharmaceutical formulations comprising monoclonal anti-Factor D antibodies, and their production and use for the treatment of complement-associated ocular diseases.

In one aspect, the anti-Factor D antibody present in the formulations is a humanized monoclonal anti-Factor D antibody. Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al. (1986) Nature 321:522-525; Riechmann et al. (1988) Nature 332:323-327; Verhoeyen et al. (1988) Science 239:1534-1536), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies can in some instances be important to reduce antigenicity and/or HAMA response (human anti-mouse antibody) when the antibody is intended for human therapeutic use. Reduction or elimination of a HAMA response is generally a significant aspect of clinical development of suitable therapeutic agents. See, e.g., Khaxzaeli et al. (1988) J. Natl. Cancer Inst 80:937; Jaffers et al. (1986) Transplantation 41:572; Shawler et al. (1985) J. Immunol. 135:1530; Sears et al. (1984) J. Biol. Response Mod. 3:138; Miller et al. (1983) Blood 62:988; Hakimi et al. (1991) J. Immunol. 147:1352; Reichmann et al. (1988) Nature 332:323; Junghans et al. (1990) Cancer Res. 50:1495. As described herein, the invention provides antibodies that are humanized such that HAMA response is reduced or eliminated. Variants of these antibodies can further be obtained using routine methods known in the art, some of which are further described below. According to the so-called “best-fit” method, the sequence of the variable domain of a rodent antibody is screened against the entire library of known human variable domain sequences. The human V domain sequence which is closest to that of the rodent is identified and the human framework region (FR) within it accepted for the humanized antibody (Sims et al. (1993) J. Immunol. 151:2296; Chothia et al. (1987) J. Mol. Biol. 196:901). Another method uses a particular framework region derived from the consensus sequence of all human antibodies of a particular subgroup of light or heavy chains. The same framework may be used for several different humanized antibodies (Carter et al. (1992) Proc. Natl. Acad. Sci. USA 89:4285; Presta et al. (1993) J. Immunol. 151:2623).

For example, an amino acid sequence from an antibody as described herein can serve as a starting (parent) sequence for diversification of the framework and/or hypervariable sequence(s). A selected framework sequence to which a starting hypervariable sequence is linked is referred to herein as an acceptor human framework. While the acceptor human frameworks may be from, or derived from, a human immunoglobulin (the VL and/or VH regions thereof), the acceptor human frameworks may be from, or derived from, a human consensus framework sequence as such frameworks have been demonstrated to have minimal, or no, immunogenicity in human patients. An “acceptor human framework” for the purposes herein is a framework comprising the amino acid sequence of a VL or VH framework derived from a human immunoglobulin framework, or from a human consensus framework. An acceptor human framework “derived from” a human immunoglobulin framework or human consensus framework may comprise the same amino acid sequence thereof, or may contain pre-existing amino acid sequence changes. Where pre-existing amino acid changes are present, preferably no more than 5 and preferably 4 or less, or 3 or less, pre-existing amino acid changes are present. In some embodiments, the VH acceptor human framework is identical in sequence to the VH human immunoglobulin framework sequence or human consensus framework sequence. In some embodiments, the VL acceptor human framework is identical in sequence to the VL human immunoglobulin framework sequence or human consensus framework sequence. A “human consensus framework” is a framework which represents the most commonly occurring amino acid residue in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al. In some embodiments, for the VL, the subgroup is subgroup kappa I as in Kabat et al. In some embodiments, for the VH, the subgroup is subgroup III as in Kabat et al.

Where the acceptor is derived from a human immunoglobulin, one may optionally select a human framework sequence that is selected based on its homology to the donor framework sequence by aligning the donor framework sequence with various human framework sequences in a collection of human framework sequences, and select the most homologous framework sequence as the acceptor. The acceptor human framework may be from or derived from human antibody germline sequences available in the public databases.

In some embodiments, human consensus frameworks herein are from, or derived from, VH subgroup VII and/or VL kappa subgroup I consensus framework sequences.

In some embodiments, the human framework template used for generation of an anti-Factor D antibody may comprise framework sequences from a template comprising a combination of VI-4.1b+ (VH7 family) and JH4d for VH chain and/or a combination of DPK4 (WI family) and JK2 for VL chain.

While the acceptor may be identical in sequence to the human framework sequence selected, whether that be from a human immunoglobulin or a human consensus framework, the acceptor sequence may also comprise pre-existing amino acid substitutions relative to the human immunoglobulin sequence or human consensus framework sequence. These pre-existing substitutions are preferably minimal; usually four, three, two or one amino acid differences only relative to the human immunoglobulin sequence or consensus framework sequence.

Hypervariable region residues of the non-human antibody are incorporated into the VL and/or VH acceptor human frameworks. For example, one may incorporate residues corresponding to the Kabat CDR residues, the Chothia hypervariable loop residues, the Abm residues, and/or contact residues. Optionally, the extended hypervariable region residues as follows are incorporated: 24-36 or 24-34 (L1), 46-56 or 50-56 (L2) and 89-97 (L3), 26-35B (H1), 50-65, 47-65 or 49-65 (H2) and 93-102, 94-102, or 95-102 (H3).

The antibodies herein include all classes and subclasses of immunoglobulin molecules, including IgG1, IgG2, IgG3, and IgG4; IgG1 antibodies being preferred.

In some embodiments, the anti-Factor D antibody present in the formulations herein is an antigen-binding fragment of a humanized anti-Factor D antibody, such as, for example, a Fab fragment or a F(ab′)₂ fragment, preferably a Fab fragment.

Fab antibody fragments provide the advantage of small size, short serum half-life, and lack of effector function, which are beneficial in many therapeutic applications. Thus, Fab molecules are advantageous when transient systemic activity that does not persist past dosing is desired or when administration and activity are localized to a peripheral compartment such as the eye. It is known, however, that several proteases cleave antibodies in the hinge-region of IgG1 antibodies, which results in anti-hinge antibodies (AHA) towards the neoepitopes. Pre-existing AHA in serum can act as surrogate Fc and reintroduce the properties of the Fc lacking in antibody fragments, which is undesirable.

A Fab molecule typically includes parts of the upper hinge of the antibody. This upper hinge region of the antibody serves as the linker between Fab and Fc region but has no structural or functional role in a Fab molecule. The recombinant expression of Fab molecules provides flexibility in defining the length of the included upper hinge region. It has been found that C-terminal truncations in the upper hinge regions and/or mutations of Fab fragments can yield neoepitopes that do not have detectable pre-existing AHA, providing a practical route to eliminate related issues.

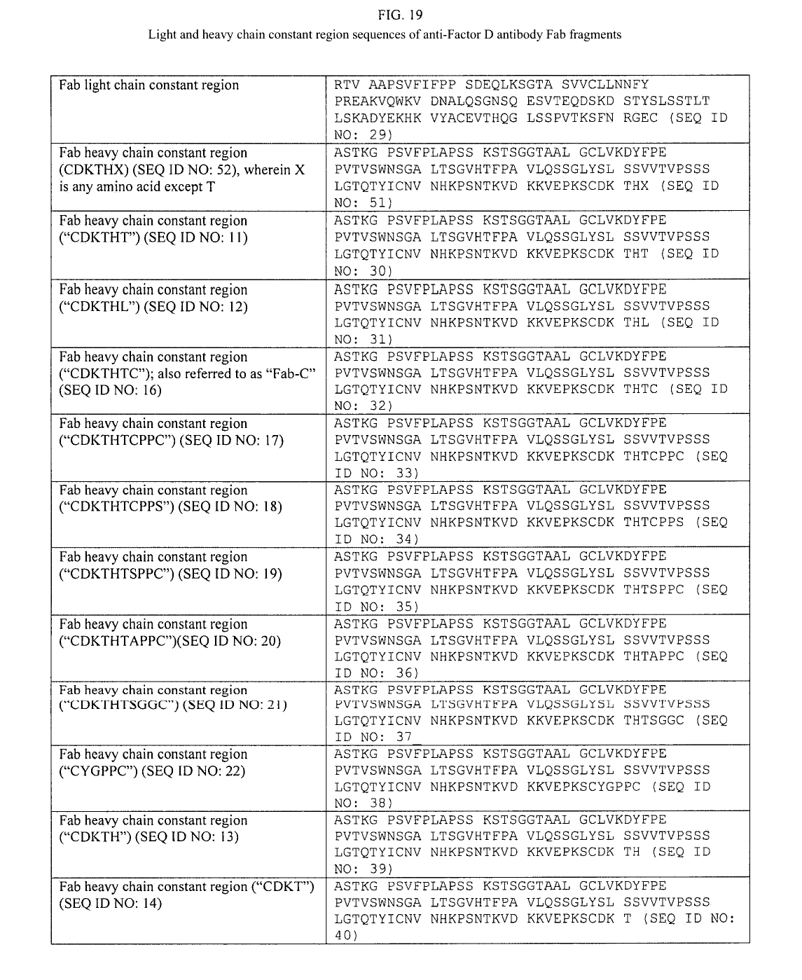

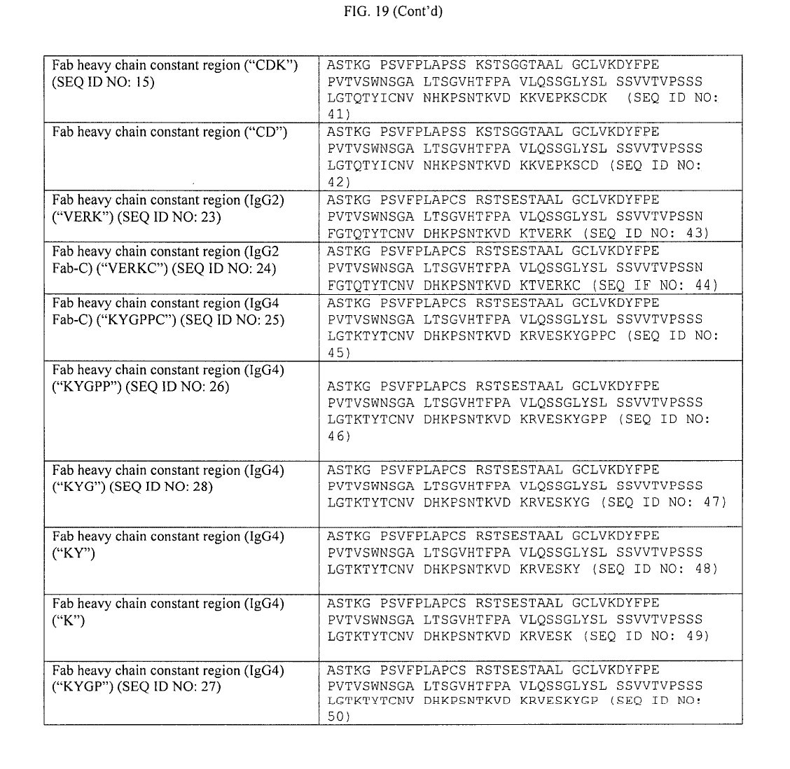

Constant region sequences of anti-Factor D antibody light and heavy chains, including heavy chains with C-terminal truncations are shown in FIG. 19.

The light chain constant region sequence of an anti-Factor D antibody Fab fragment is shown in FIG. 19 as SEQ ID NO: 29. In some embodiments, the C-terminus of the heavy chain of the Fab fragment ends in the sequence CDKTHX (SEQ ID NO: 52), wherein X is any amino acid except T. The present invention specifically includes formulations comprising anti-Factor D antibodies with the C-terminal terminus of the heavy chain of a Fab fragment ending in the amino acids “CDKTHT” (SEQ ID NO: 11), “CDKTHL” (SEQ ID NO: 12), “CDKTH” (SEQ ID NO: 13), “CDKT” (SEQ ID NO: 14), “CDK” (SEQ ID NO: 15), or “CD”. Truncations and/or mutations at the C terminus are able to eliminate AHA-reactivity against the Fab, without compromising thermostability or expression. In some embodiments, the C-terminus of the heavy chain of a Fab fragment ends in the amino acids “CDKTHTC” (SEQ ID NO: 16), “CDKTHTCPPC” (SEQ ID NO: 17), “CDKTHTCPPS” (SEQ ID NO: 18), “CDKTHTSPPC” (SEQ ID NO: 19), “CDKTHTAPPC” (SEQ ID NO: 20), “CDKTHTSGGC” (SEQ ID NO: 21), or “CYGPPC” (SEQ ID NO: 22). In some such embodiments, a free cysteine in the C-terminal amino acids may be amenable to conjugation, for example, to a polymer such as PEG. In some embodiments, a Fab fragment comprises a, IgG1 heavy chain constant region selected from SEQ ID NOs: 30-42 (FIG. 19). In some embodiments, a Fab is an IgG2 or IgG4 Fab (See, e.g. SEQ ID NOs: 37-43) (FIG. 19). In some embodiments, a Fab is an IgG2 Fab fragment comprising a heavy chain constant region of SEQ ID NO: 43 (VERK; SEQ ID NO: 23) or IgG2 Fab-C fragment comprising a heavy chain constant region of SEQ ID NO: 44 (VERKC; SEQ ID NO: 24). In some embodiments, a Fab is an IgG4 fragment comprising a heavy chain constant region selected from SEQ ID NO: 46 (KYGPP; SEQ ID NO: 26), SEQ ID NO: 50 (KYGP; SEQ ID NO: 27), SEQ ID NO: 47 (KYG, SEQ ID NO: 28), SEQ ID NO: 48 (KY), and SEQ ID NO: 49 (K) or an IgG4 Fab-C fragment comprising a heavy chain constant region of SEQ ID NO: 45 (KYGPPC; SEQ ID NO: 25). As an alternative to truncating and/or mutation at the C terminus, to avoid pre-existing anti-hinge antibody (PE-AHA) responses, IgG1 or IgG4 Fab fragments can be used, since these do not show PE-AHA response.

Antibodies have a variety of stability issues. The complementarity determining regions (DCRs) of antibodies are vulnerable to posttranslational modifications because of their inflexibility and accessibility to solvent. Chemical degradation due to Trp oxidation, Asn deamidation and Asp isomerization within the CDRs have been reported.

In some embodiments, the anti-Factor D antibody herein is a humanized monoclonal antibody, susceptible to isomerization of aspartyl (Asp) residues, such as antibodies comprising an Asp-Xaa motif, wherein Xaa is Asp, Gly, His, Ser or Thr, in at least one heavy and/or light chain hypervariable region (HVR).

In some embodiments, the monoclonal anti-Factor D antibody present in the formulations of this invention comprises heavy chain hypervariable regions (HVR-HCs) having at least 90%, or at least 95%, or at least 98%, or at least 99% sequence identity to the HVR sequences of HVR1-HC: GYTFTNYGMN (SEQ ID NO: 3); HVR2-HC: WINTYTGETTYADDFKG (SEQ ID NO: 4); HVR3-HC: EGGVNN (SEQ ID NO: 5) and/or light chain hypervariable regions (HVR-LCs) having at least 90%, or at least 95%, or at least 98%, or at least 99% sequence identity to the HVR-LC sequences of HVR1-LC: ITSTDIDDDMN (SEQ ID NO: 8); HVR2-LC: GGNTLRP (SEQ ID NO: 9); and HVR3-LC: LQSDSLPYT (SEQ ID NO: 10).

In some embodiments, the monoclonal anti-Factor D antibody comprises the HVR-HCs of HVR1-HC: GYTFTNYGMN (SEQ ID NO: 3); HVR2-HC: WINTYTGETTYADDFKG (SEQ ID NO: 4); HVR3-HC: EGGVNN (SEQ ID NO: 5) and/or the HVR-LC of HVR1-LC: ITSTDIDDDMN (SEQ ID NO: 8); HVR2-LC: GGNTLRP (SEQ ID NO: 9); and HVR3-LC: LQSDSLPYT (SEQ ID NO: 10).

In some embodiments, the monoclonal anti-Factor D antibody comprises a heavy chain variable region sequence having at least 85%, or at least 90%, or at least 95%, or at least 98%, or at least 99% sequence identity to the variable region sequence of the heavy chain of SEQ ID NO: 2 and/or a light chain variable region sequence having at least 85%, or at least 90%, or at least 95%, or at least 98%, or at least 99% sequence identity to the variable region sequence of the light chain of SEQ ID NO: 7.

In some embodiments, the monoclonal anti-Factor D antibody comprises the variable region sequence of the heavy chain of SEQ ID NO: 2 and/or the variable region sequence of the light chain of SEQ ID NO: 7.

In some embodiments, the monoclonal anti-Factor D antibody comprises a heavy chain sequence comprising SEQ ID NO: 2 and/or a light chain sequence comprising SEQ ID NO: 7.

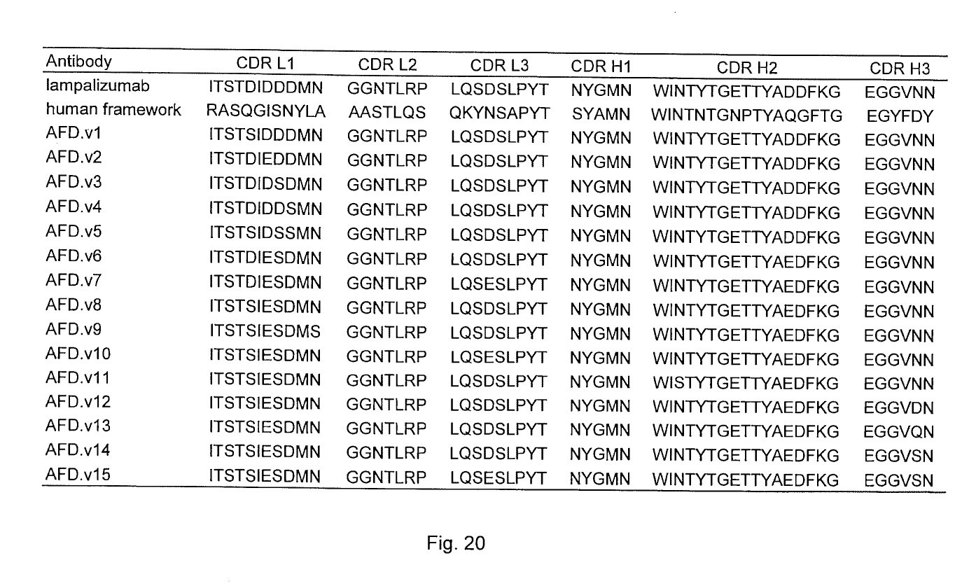

In some embodiments, the monoclonal anti-Factor D antibody present in the formulations of this invention comprises heavy chain hypervariable regions (HVR-HCs) having at least 90%, or at least 95%, or at least 98%, or at least 99% sequence identity to the heavy and/or light chain CDR sequences of anti-Factor D antibody variants AFD.v1-AFD.v15 (see FIG. 20).

In some embodiments, the monoclonal anti-Factor D antibody comprises the heavy and/or light chain CDR sequence of anti-Factor D antibody variants AFD.v1-AFD.v15 (see FIG. 20).

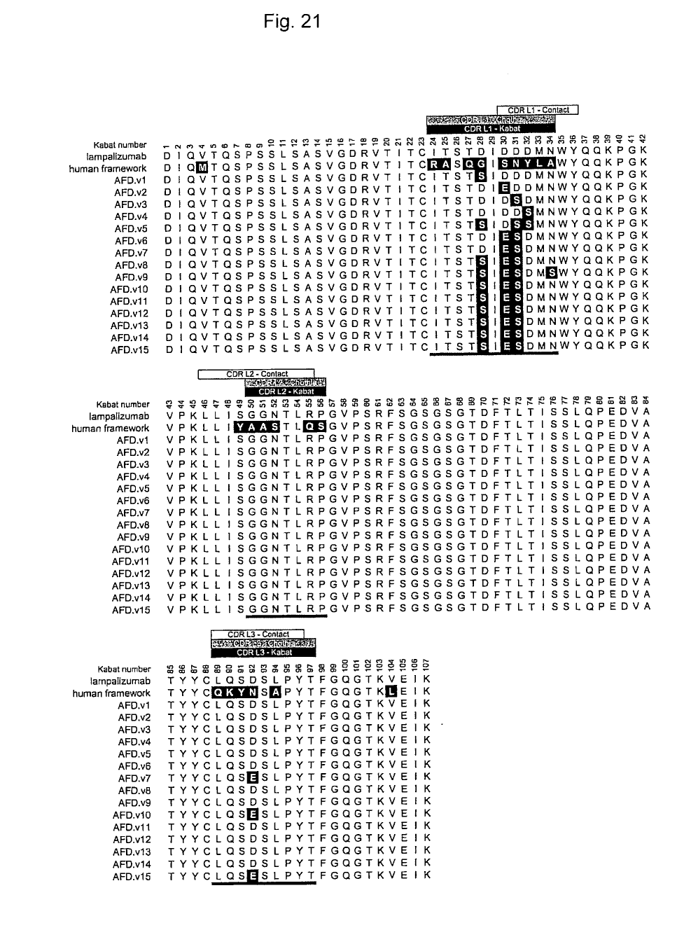

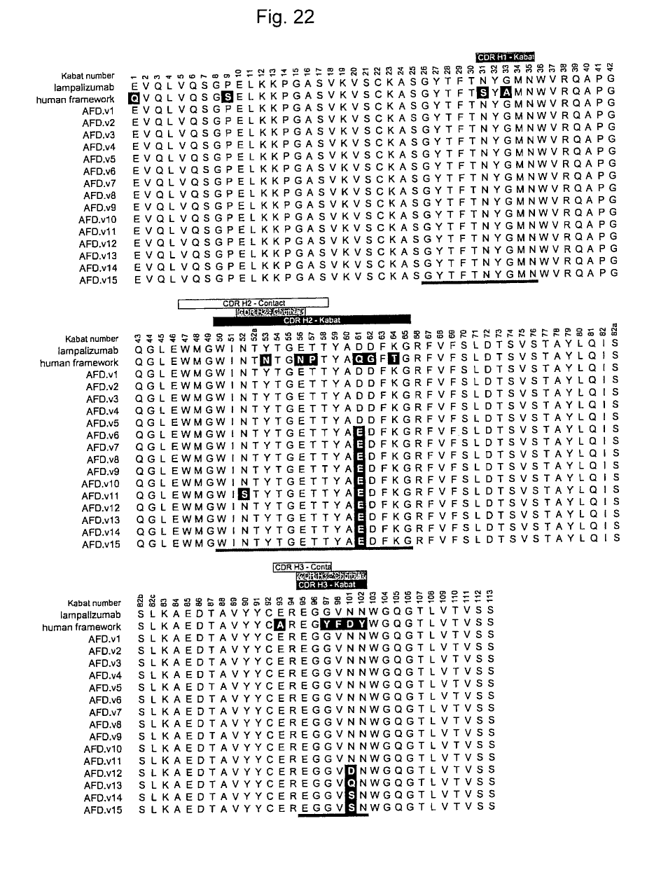

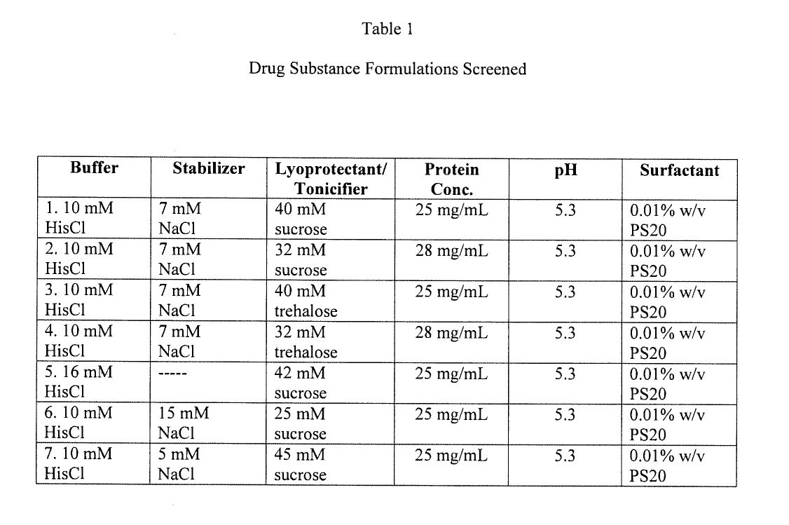

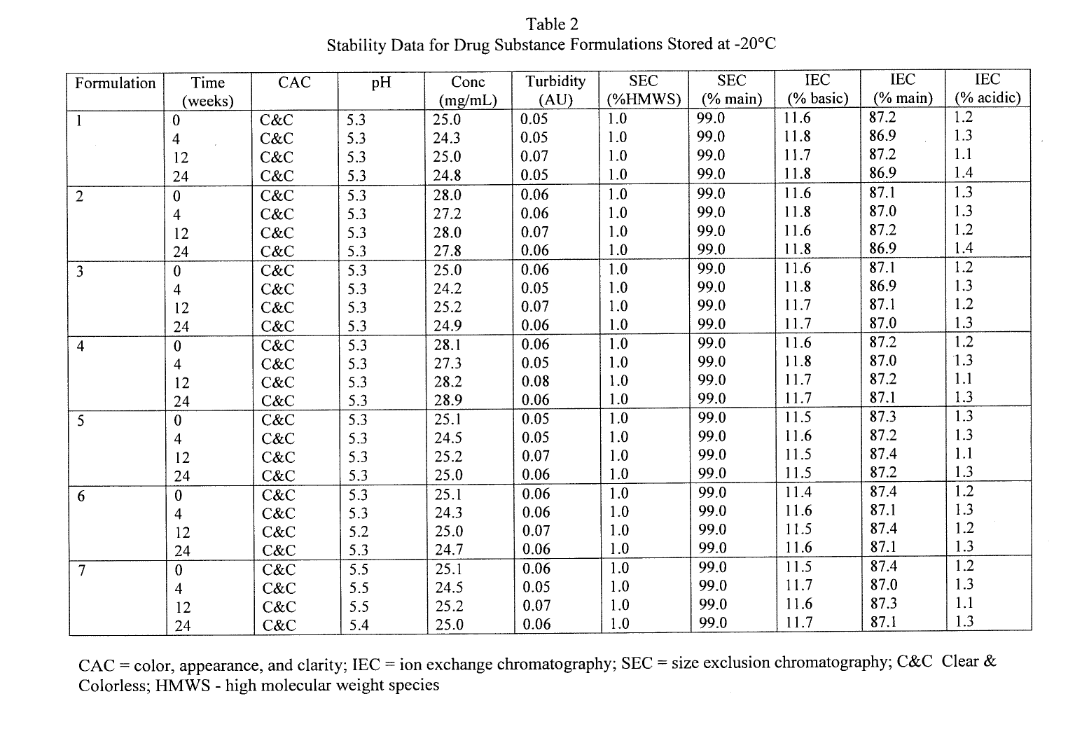

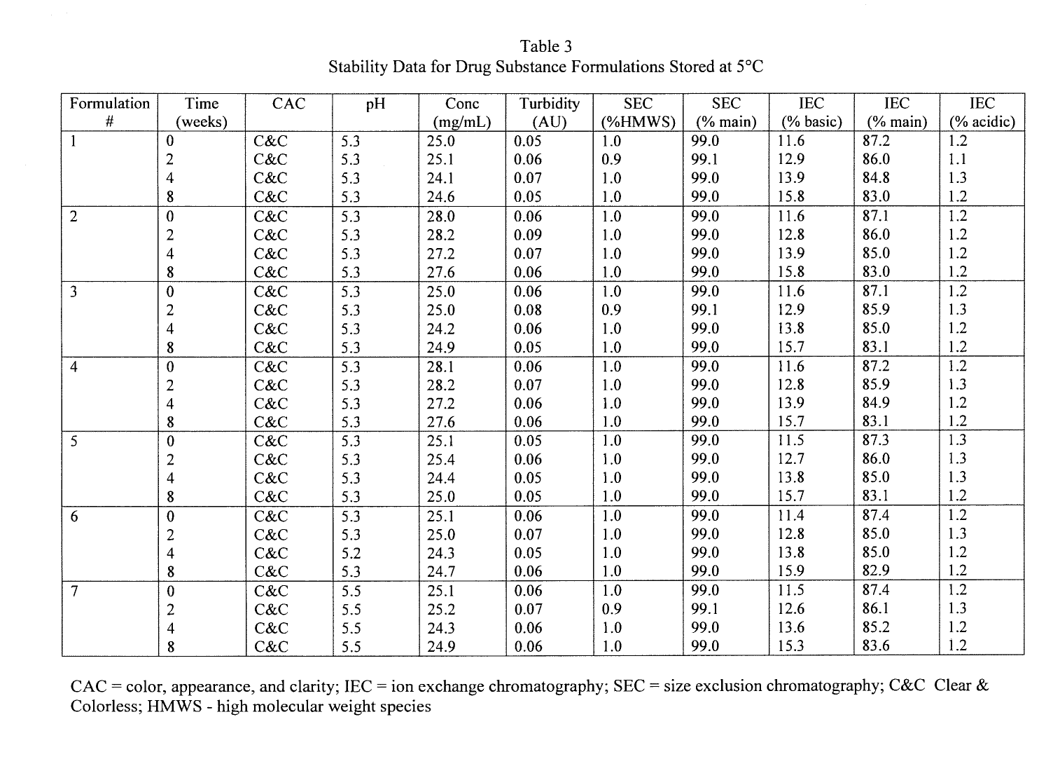

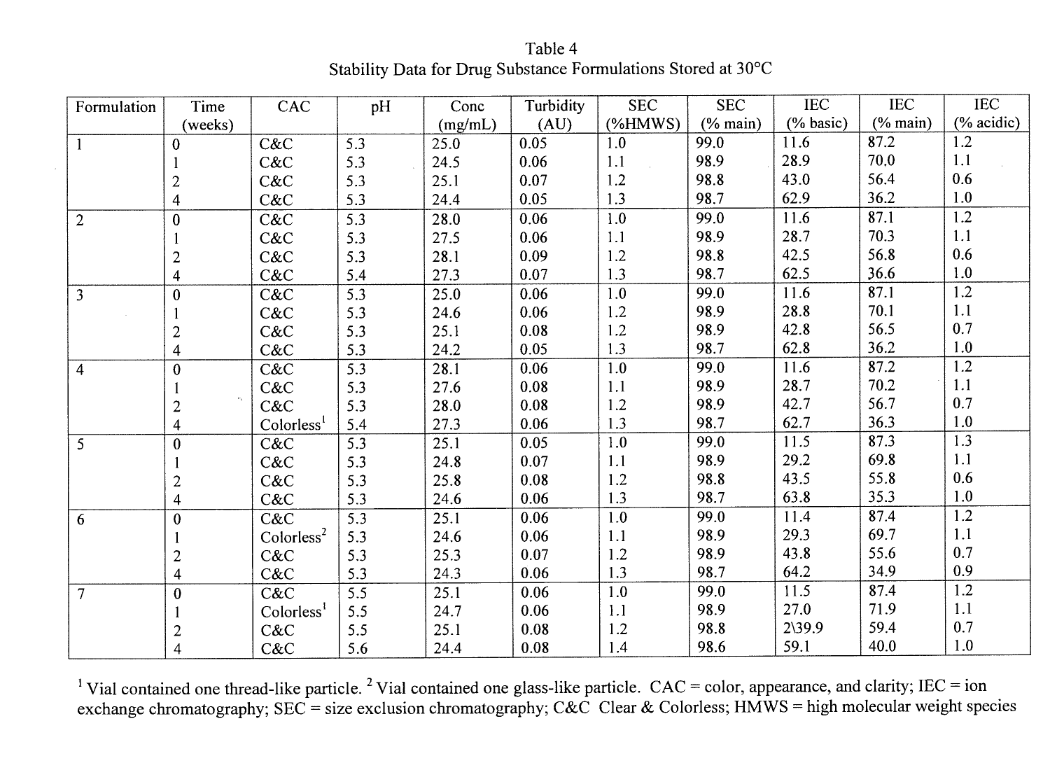

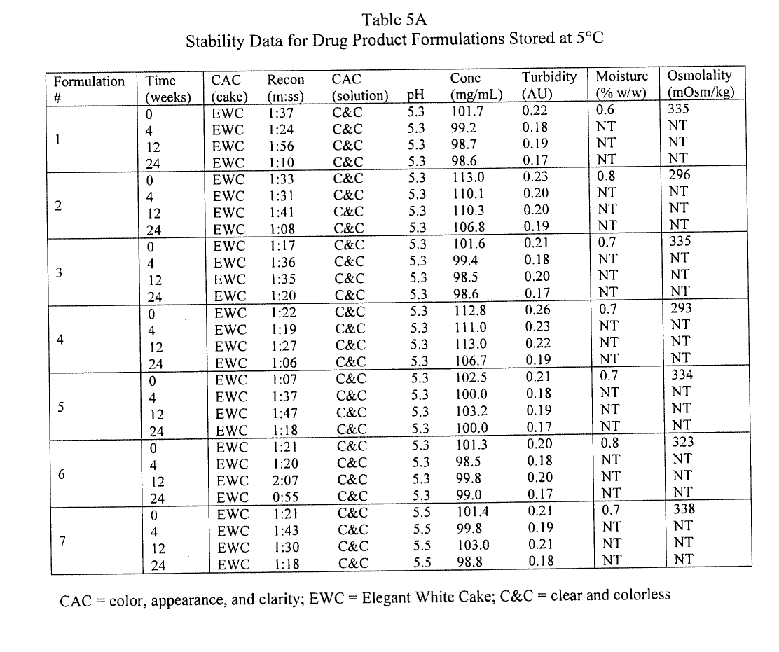

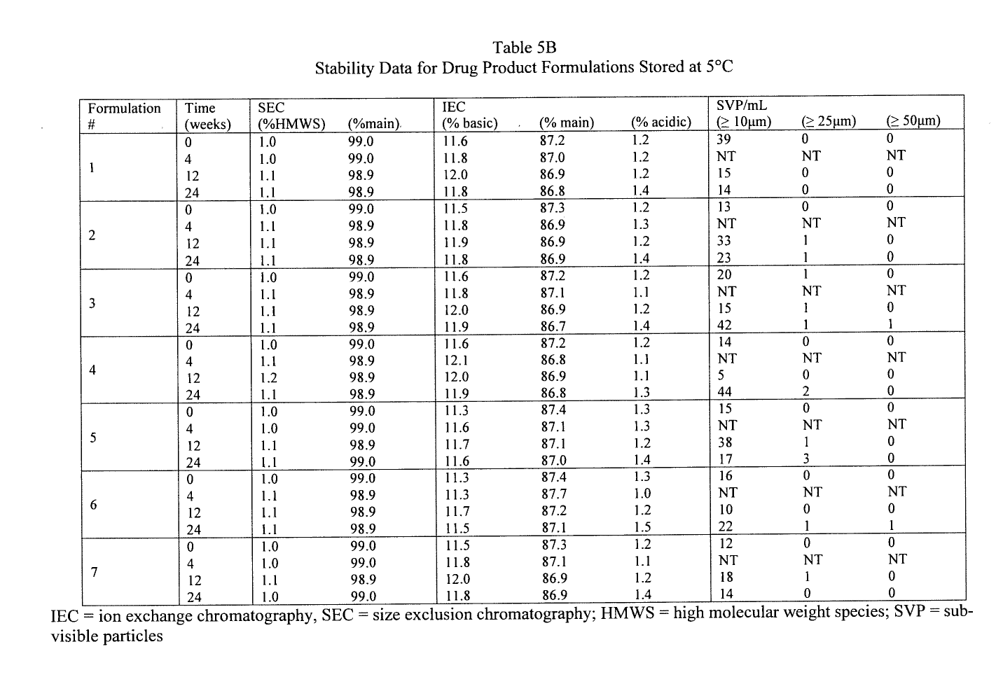

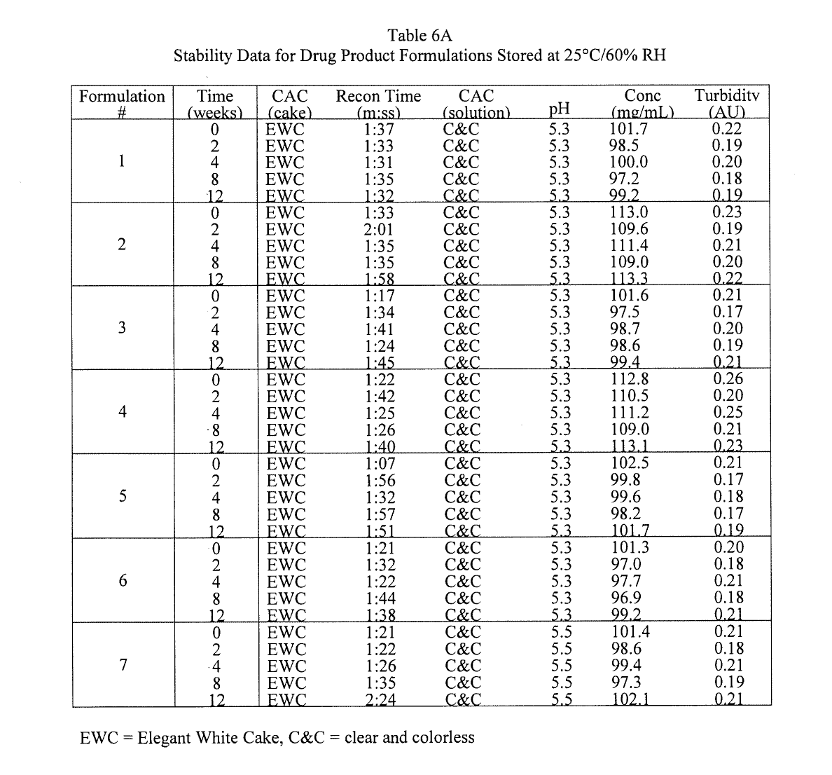

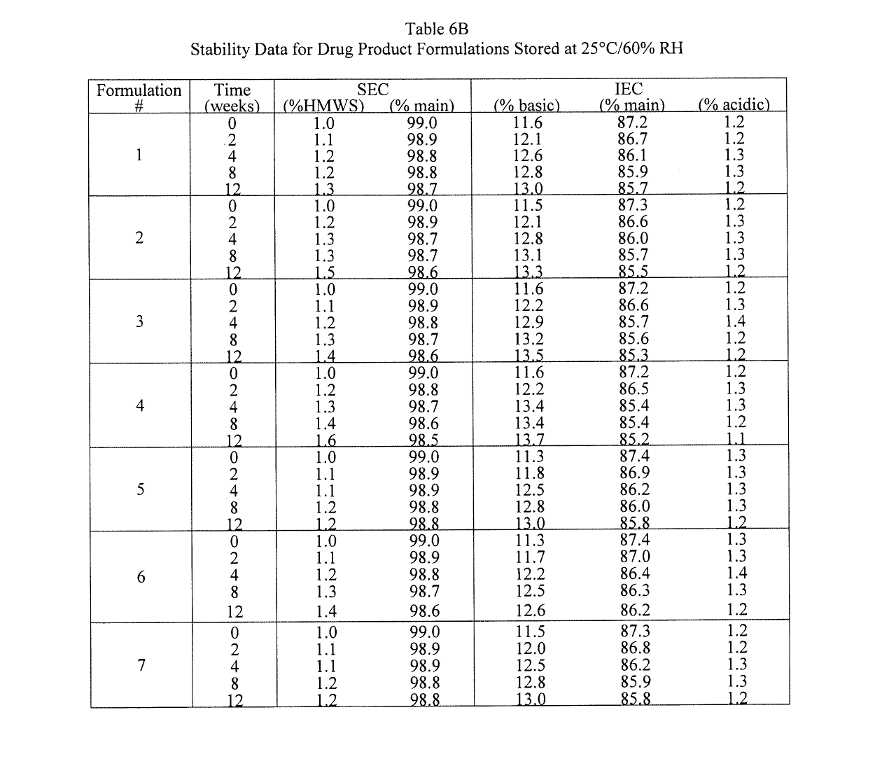

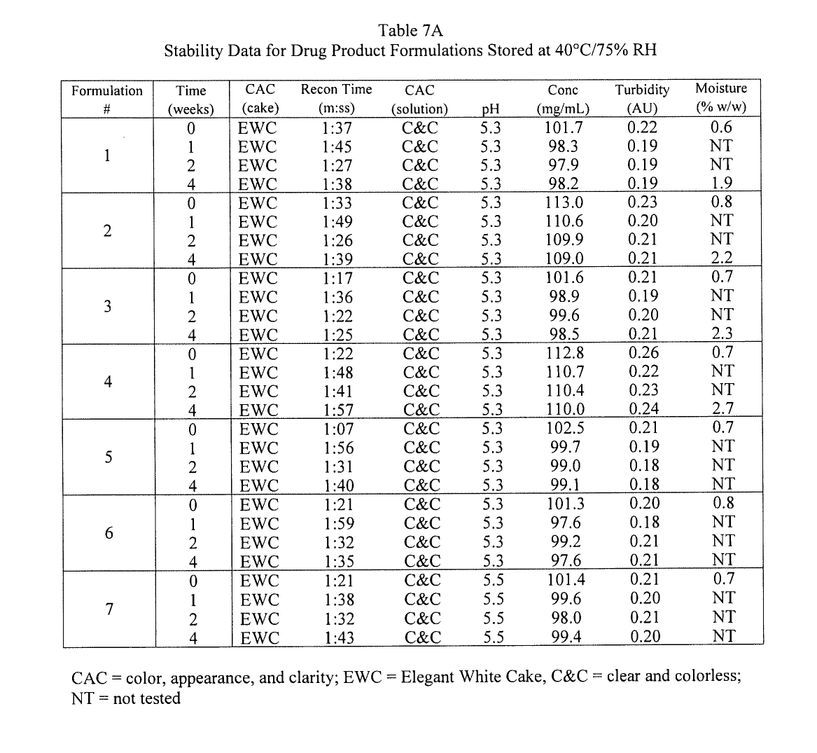

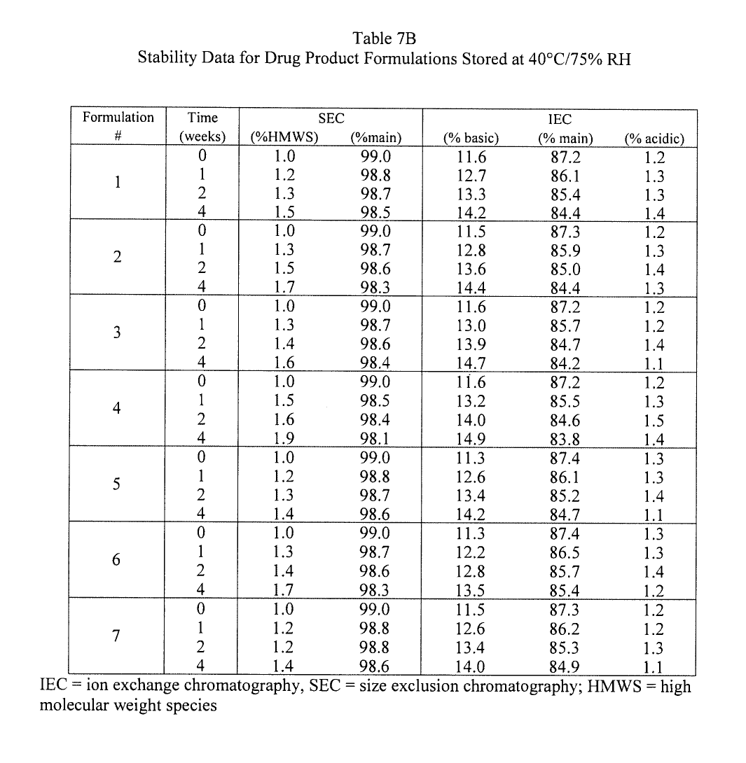

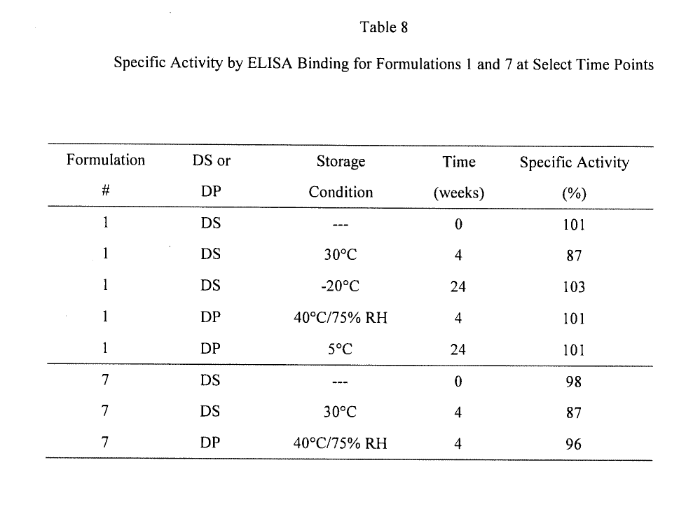

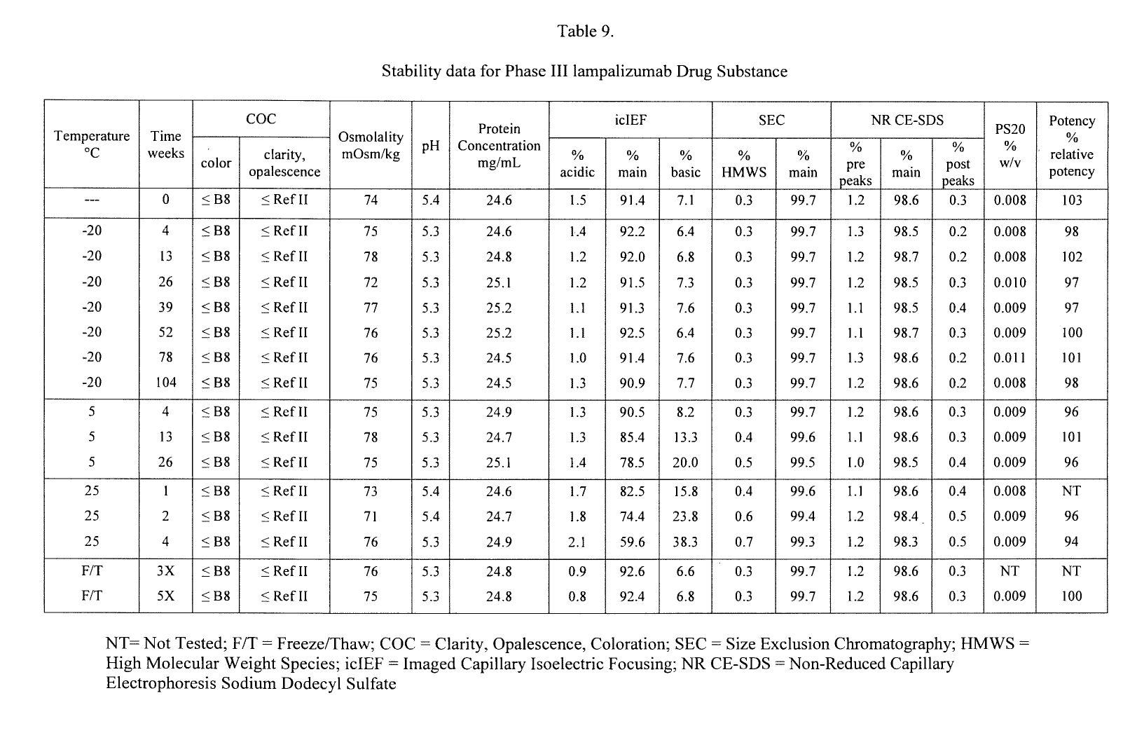

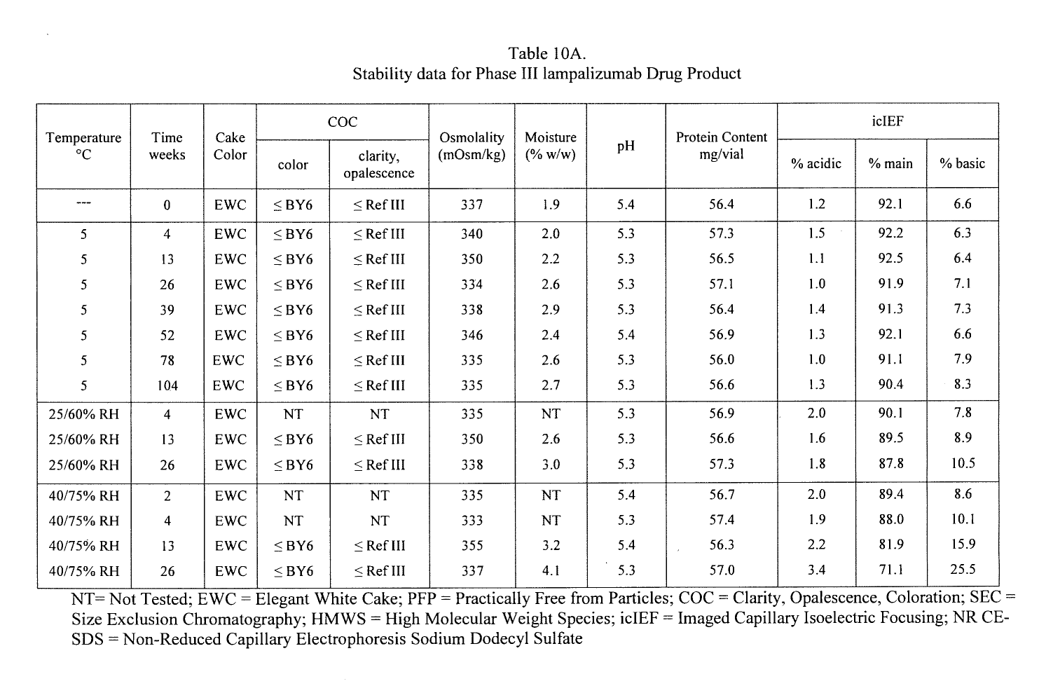

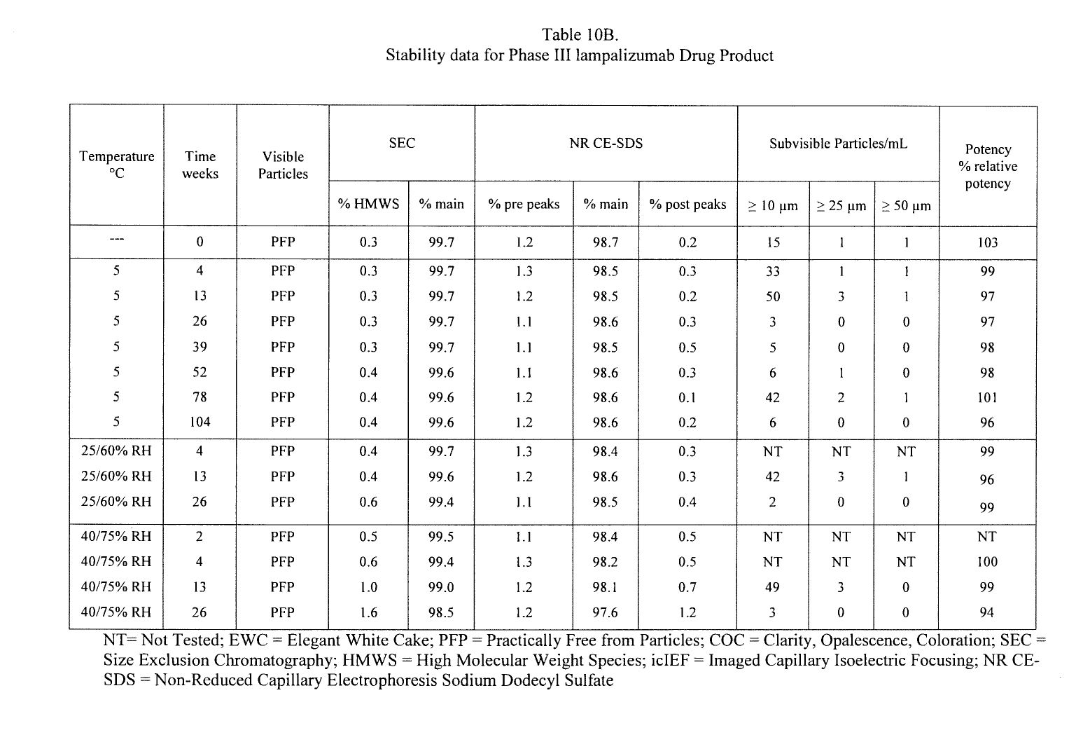

In some embodiments, the monoclonal anti-Factor D antibody comprises a heavy chain variable region sequence having at least 85%, or at least 90%, or at least 95%, or at least 98%, or at least 99% sequence identity to the variable region sequence of the light chain and/or heavy chain of anti-Factor D antibody variants AFD.v1-AFD.v15 (see FIGS. 21 and 22).

In some embodiments, the monoclonal anti-Factor D antibody comprises the light chain and/or heavy chain variable region sequence of anti-Factor D antibody variants AFD.v1-AFD.v15 (see FIGS. 21 and 22).

In some embodiments, the C-terminus of the heavy chain of the Fab fragment ends in the sequence CDKTHX (SEQ ID NO: 52), wherein X is any amino acid except T. The present invention specifically includes formulations comprising anti-Factor D antibody variants (e.g. AFD.v1-AFD.v15) with the C-terminal terminus of the heavy chain of a Fab fragment ending in the amino acids “CDKTHT” (SEQ ID NO: 11), “CDKTHL” (SEQ ID NO: 12), “CDKTH” (SEQ ID NO: 13), “CDKT” (SEQ ID NO: 14), “CDK” (SEQ ID NO: 15), or “CD”. As discussed above, truncations and/or mutations at the C terminus are able to eliminate AHA-reactivity against the Fab, without compromising thermostability or expression. In some embodiments, the C-terminus of the heavy chain of a Fab fragment of an anti-Factor D antibody variant (e.g. AFD.v1-AFD.v15) ends in the amino acids “CDKTHTC” (SEQ ID NO: 16), “CDKTHTCPPC” (SEQ ID NO: 17), “CDKTHTCPPS” (SEQ ID NO: 18), “CDKTHTSPPC” (SEQ ID NO: 19), “CDKTHTAPPC” (SEQ ID NO: 20), “CDKTHTSGGC” (SEQ ID NO: 21), or “CYGPPC” (SEQ ID NO: 22). In some such embodiments, a free cysteine in the C-terminal amino acids may be amenable to conjugation, for example, to a polymer such as PEG. In some embodiments, a Fab fragment comprises an IgG1 heavy chain constant region selected from SEQ ID NOs: 30 to 42. In some embodiments, a Fab is an IgG2 or IgG4 Fab (See, e.g. SEQ ID NOs: 43 to 51) (FIG. 19). Thus, in some embodiments, a Fab is an IgG2 Fab fragment comprising a heavy chain constant region of SEQ ID NO: 43 (VERK; SEQ ID NO: 23) or IgG2 Fab-C fragment comprising a heavy chain constant region of SEQ ID NO: 44 (VERKC; SEQ ID NO: 24). In some embodiments, a Fab is an IgG4 fragment comprising a heavy chain constant region selected from SEQ ID NO: 46 (KYGPP, SEQ ID NO: 26), SEQ ID NO: 50 (KYGP; SEQ ID NO: 27), SEQ ID NO: 47 (KYG; SEQ ID NO: 28), SEQ ID NO: 48 (KY), and SEQ ID NO: 49 (K) or an IgG4 Fab-C fragment comprising a heavy chain constant region of SEQ ID NO: 45 (KYGPPC; SEQ ID NO: 25).

As an alternative to truncating and/or mutation at the C terminus, to avoid pre-existing anti-hinge antibody (PE-AHA) responses, IgG1 or IgG4 Fab fragments can be used, since these do not show PE-AHA response.

In some embodiments the anti-Factor D antibody is lampalizumab.

In some embodiments, the antibody is anti-Factor D antibody variant AFD.v8 or AFD.v14.

The anti-Factor D antibodies included in the formulations of the present invention, including the anti-Factor D variants herein, can also be further covalently modified by conjugating the antibody to one of a variety of non-proteinacious polymer molecules. The antibody-polymer conjugates can be made using any suitable technique for derivatizing antibody with polymers. It will be appreciated that the invention is not limited to conjugates utilizing any particular type of linkage between an antibody or antibody fragment and a polymer.

In one aspect, the conjugates of the invention include species wherein a polymer is covalently attached to a specific site or specific sites on the parental antibody, i.e. polymer attachment is targeted to a particular region or a particular amino acid residue or residues in the parental antibody or antibody fragment. Site specific conjugation of polymers is most commonly achieved by attachment to cysteine residues in the parental antibody or antibody fragment. In such embodiments, the coupling chemistry can, for example, utilize the free sulfhydryl group of a cysteine residue not in a disulfide bridge in the parental antibody. The polymer can be activated with any functional group that is capable of reacting specifically with the free sulfhydryl or thiol group(s) on the parental antibody, such as maleimide, sulfhydryl, thiol, triflate, tesylate, aziridine, exirane, and 5-pyridyl functional groups. The polymer can be coupled to the parental antibody using any protocol suitable for the chemistry of the coupling system selected, such as the protocols and systems described in U.S. Pat. Nos. 4,179,337; 7,122,636, and Jevsevar et al. (2010) Biotech. J. 5:113-128.

In some embodiments, one or more cysteine residue(s) naturally present in the parental antibody is (are) used as attachment site(s) for polymer conjugation. In some embodiments, one or more cysteine residue(s) is (are) engineered into a selected site or sites in the parental antibody for the purpose of providing a specific attachment site or sites for polymer.

In one aspect, the invention encompasses formulations comprising antibody fragment-polymer conjugates, wherein the antibody fragment is a Fab, and the polymer is attached to one or more cysteine residue in the light or heavy chain of the Fab fragment that would ordinarily form the inter-chain disulfide bond linking the light and heavy chains.

In another aspect, the invention encompasses formulations comprising antibody fragment-polymer conjugates, wherein the antibody fragment is a Fab′ (includes Fab-C), and the polymer attachment is targeted to the hinge region of the Fab′ fragment (includes Fab-C). In some embodiments, one or more cysteine residue(s) naturally present in the hinge region of the antibody fragment is (are) used to attach the polymer. In some embodiments, one or more cysteine residues is (are) engineered into the hinge region of the Fab′ fragment (includes Fab-C) for the purpose of providing a specific attachment site or sites for polymer. Cysteine engineered antibodies have been described previously (U.S. Pat. Pub. No. 2007/0092940 and Junutula, J. R., et al, J. Immunol Methods, Vol. 332(1-2), pp. 41-52 (2008), all herein incorporated by reference in their entirety). In some embodiments, cysteine engineered antibodies can be parental antibodies. These are useful for generating antibody fragments having a free cysteine in a particular location, typically in a constant region, e.g., CL or CH1. A parent antibody engineered to contain a cysteine may be referred to as a “ThioMab” and Fab fragments produced from such cysteine engineered antibodies, regardless of the method of production, may be referred as “ThioMabs” or “ThioFabs.” As described previously (see, e.g., U.S. Pat. Pub. No. 2007/0092940 and Junutula, J. R., et al, J. Immunol Methods, Vol. 332(1-2), pp. 41-52 (2008)), mutants with replaced (“engineered”) cysteine (Cys) residues are evaluated for the reactivity of the newly introduced, engineered cysteine thiol groups. The thiol reactivity value is a relative, numerical term in the range of 0 to 1.0 and can be measured for any cysteine engineered antibody. In addition to having a reactive thiol group, ThioMabs should be selected such that they retain antigen binding capability. The design, selection, and preparation of cysteine engineered antibodies were described in detail previously (see, e.g., WO 2011/069104, which is herein incorporated by reference). Engineered cysteines are preferably introduced into the constant domains of heavy or light chains. As such, the cysteine engineered antibodies will preferably retain the antigen binding capability of their wild type, parent antibody counterparts and, as such, are capable of binding specifically, to antigens. In some embodiments, the anti-Factor D antibody variant Fab fragment of the invention is modified by adding one cysteine at the C′-terminal end for the purpose of providing one attachment site for polymer conjugation. In another some embodiments, the anti-Factor D antibody variant Fab fragment of the invention is modified by adding four additional residues, Cys-Pro-Pro-Cys (SEQ ID NO: 53), at the C′-terminal end for the purpose of providing two attachment sites for polymer conjugation.

One commonly used antibody conjugation is PEGylation, wherein one or more polyethylene glycol (PEG) polymers are covalently attached to the antibody's constant region. See U.S. Pat. Nos. 4,179,337; 7,122,636. PEG polymers of different sizes (e.g., from about 500 D to about 300,000 D) and shapes (e.g., linear or branched) have been known and widely used in the field. The polymers useful for the present invention may be obtained commercially (e.g., from Nippon Oil and Fats; Nektar Therapeutics; Creative PEGWorks) or prepared from commercially available starting materials using conventional chemical procedures. PEGylation changes the physical and chemical properties of the antibody drug, and may results in improved pharmacokinetic behaviors such as improved stability, decreased immunogenicity, extended circulating life as well as increased residence time.

As discussed above, most preferably the anti-Factor D antibody is lampalizumab.

Lampalizumab is an antigen-binding fragment (Fab) of a humanized anti-Factor D monoclonal antibody based on a human IgG1 isotype. Lampalizumab is produced in Escherichia coli (E. coli) and consists of one partial heavy chain and one light chain comprising inter- and intra-chain disulfide bonds. Lampalizumab is directed against the complement Factor D. Factor D is a highly specific chymotrypsin-like serine protease that is a rate-limiting enzyme in the activation of the alternative complement pathway. The substrate for Factor D is another alternative pathway serine protease, Factor B. Following cleavage by Factor D, Factor B converts into the proteolytically active factor Bb and initiates the alternative complement pathway. Increased activation of the alternative complement pathway has been found in drusen, cytotoxic deposits present on the Bruch's membrane which are associated with the development of age-related macular degeneration (AMD). (Despriet D D, et al., (2006). Complement factor H polymorphism, complement activators, and risk of age-related macular degeneration. JAMA 296 (3): 301-9. A role of alternative pathway complement activation in AMD has further been supported by genetics, showing that a mutation in Factor H, a negative regulator of alternative complement pathway activation, is strongly correlated with increased risk for developing AMD. Lampalizumab activity is specific for the alternative pathway and shows no inhibitory effect on classical pathway activation. Lampalizumab inhibits Factor D-mediated cleavage of Factor B, preventing alternative complement pathway activation, and thereby inhibiting inflammation and cytotoxic activity of the activated complement components (Atkinson JP and Frank MM (2006). Bypassing Complement: Evolutionary Lessons and Future Implications. J Clin Invest 116(5):1215-18).

A pharmaceutical composition comprising lampalizumab Drug Product (DP) as a sterile, white to off-white, lyophilized powder in a 6-cc USP/Ph. Eur. Type 1 glass vial intended for ITV administration is described in WO2015/023596. Each glass vial contained nominal 40 mg of lampalizumab. Reconstitution of the Drug Product with sterile water for injection (SWFI), USP/Ph. Eur., was required. After reconstitution, the Drug Product was formulated as 100 mg/mL lampalizumab in 40 mM L-histidine hydrochloride, 20 mM sodium chloride, 180 mM sucrose, 0.04% (w/v) polysorbate 20, pH 5.5. The Drug Product contained no preservatives and was suitable for single use only.

In one aspect, the present invention concerns improved lampalizumab formulations, including pre-lyophilized, lyophilized and reconstituted formulations.

One problem addressed by the present invention is that the pH of the vitreous is around 7.4 and this has to be balanced with the highest acceptable pH for lampalizumab formulations. Indeed, lampalizumab has limited solubility at the higher end of the acceptable pH range (around pH 5.8). Solubility could be improved by increasing the ionic strength or reducing the pH of the formulation. However, the pH must stay within relatively narrow limits since injecting acidic solutions into the human vitreous raises safety issues. Solubility may also be improved by increasing the concentration of NaCl in the formulation without reducing the pH. However, one additional mM of NaCl in the formulation would remove approximately two mM of sucrose to maintain tonicity. Previous studies have shown that the aggregation rate of lyophilized proteins is significantly higher when formulated with lower sugar-to-protein ratios. (Cleland, J L et al. (2001). A Specific Molar Ratio of Stabilizer to Protein is Required for Storage Stability of a Lyophilized Monoclonal Antibody. J Pharm Sci: 90(3):310-21). Thus, this approach would result in a sub-optimal concentration of sugar because the DP must be approximately isotonic to be considered safe for intravitreal administration. Therefore, to maintain a suitable sugar-to-protein ratio and solubility and to meet safety requirements, other ways had to be explored for maintaining DP stability while simultaneously improving lampalizumab solubility.

The route of administration not only places constraints on the osmolality and pH of the formulation, it also limits the excipient species that can be used. For example, components like histidine acetate are not suitable for use in the formulations of the present invention, which undergo lyophilization prior to reconstitution, because acetic acid is volatile and could be removed from the formulation during lyophilization.

The present invention provides improved pharmaceutical formulations of anti-Factor D antibodies suitable for intraocular, preferably intravitreal, administration, comprising an anti-Factor D antibody at a pH below 5.5 and yet suitable or intravitreal administration. Preferably, the pH is 5.0, 5.1, 5.2, 5.3 or 5.4. The formulations can include any buffer which provides the formulation at a suitable pH, preferably excluding the use of dual buffers, such as phosphate/citrate buffers. Exemplary suitable buffers include sodium citrate, sodium succinate and histidine buffers. For the purpose of the present invention, a histidine buffer is preferred, which can not only provide the required pH but also has lyoprotective properties.

In some embodiments, the anti-Factor D antibody formulations undergo lyophilization and are reconstituted prior to administration. Thus the formulations herein preferably include one or more lyoprotactants. Lyoprotectants include polyols (sugars), as defined above, such as sucrose or trehalose; an amino acid such as monosodium glutamate or histidine; a methylamine such as betaine; a lyotropic salt such as magnesium sulfate; a polyol such as trihydric or higher sugar alcohols, e.g. glycerin, erythritol, glycerol, arabitol, xylitol, sorbitol, and mannitol; propylene glycol; polyethylene glycol; Pluronics; and combinations thereof. The preferred lyoprotectant is a non-reducing polyol, such as trehalose or sucrose, preferably sucrose.

The formulations herein may also include one or more bulking agents, i.e. a compound which adds mass to the lyophilized mixture and contributes to the physical structure of the lyophilized cake (e.g. facilitates the production of an essentially uniform lyophilized cake which maintains an open pore structure). Exemplary bulking agents include mannitol, glycine, polyethylene glycol and xorbitol.

The formulation herein may further include one or more surfactants (e.g. a polysorbate) in that it has been observed herein that this can reduce aggregation of the reconstituted protein and/or reduce the formation of particulates in the reconstituted formulation. The surfactant can be added to the pre-lyophilized formulation, the lyophilized formulation and/or the reconstituted formulation (but preferably the pre-lyophilized formulation) as desired.

Since the reconstituted formulations are not intended for long term storage, presence of a preservative is generally not required in the formulations herein. It is, however, possible to prepare formulations comprising a preservative. Examples of potential preservatives include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3-pentanol, and m-cresol. The most preferred preservative herein is benzyl alcohol.

In some embodiments, the formulations herein comprise a monoclonal anti-Factor D antibody, a buffer suitable for adjusting the pH in the range of 5.0-5.4, a polyol, and a surfactant. Preferably, the pH is about 5.3, the buffer is a histidine buffer, the polyol is sucrose, and the surfactant is a polysorbate.

In some embodiments, the same ingredients are present in the pre-lyophilized, lyophilized, and reconstituted formulations.

In some embodiments, the pre-lyophilized formulation comprises about 25 mg/mL anti-Factor D antibody.

In some embodiments, the reconstituted formulation comprises about 100 mg/ml anti-Factor D antibody.

Use of the Anti-Factor D Antibody Formulations

The formulations of the present invention, which comprise antibodies recognizing Factor D as their target, may be used to treat complement-associated ocular disorders. Complement-associated ocular disorders include, for example, macular degenerative diseases, such as all stages of age-related macular degeneration (AMD), including dry and wet (non-exudative and exudative) forms, choroidal neovascularization (CNV), uveitis, diabetic and other ischemia-related retinopathies, endophthalmitis, and other intraocular neovascular diseases, such as diabetic macular edema, pathological myopia, von Hippel-Lindau disease, histoplasmosis of the eye, Central Retinal Vein Occlusion (CRVO), corneal neovascularization, and retinal neovascularization.

In one example, the complement-associated ocular disorders include age-related macular degeneration (AMD), including non-exudative (wet) and exudative (dry or atrophic) AMD, choroidal neovascularization (CNV), diabetic retinopathy (DR), endophthalmitis, and uveitis.

In another example, the AMD is dry AMD, including the advanced form characterized by geographic atrophy.

In one example, the complement-associated eye condition is geographic atrophy. In one example, the complement-associated eye condition is wet AMD (choroidal neovascularization (CNV)).

The anti-factor D antibody formulations herein are administered by intraocular administration, preferably intravitreal injection. A typical dose is about 10 mg per eye, administered every 4 or 6 weeks, or by every 2-6 weeks, or by every 2 weeks by intravitreal injection.

In some embodiments, the formulations herein are used to treat geographic atrophy (GA), the advanced form of age-related macular degeneration (AMD), a progressive condition which can result in blindness. Efficacy can be evaluated by determining a reduction in the rate of GA disease progression, defined as the mean change in the GA lesion area of the affected eye from baseline, as measured by known techniques, such as fundus autofluorescence (FAF), an imaging technique used to provide information about the size and type of GA lesions in the macula. Secondary efficacy endpoints focus on assessing the impact of lampalizumab treatment on patients' visual function.

The anti-Factor D formulations of the present invention may be used in combination with one or more additional therapeutic agents. In certain embodiments, an additional therapeutic agent is a therapeutic agent suitable for treatment of a complement-associated ocular disease. In some embodiments, the additional therapeutic agent is suitable for the treatment of an ocular disorder associated with undesirable neovascularization in the eye, such as, for example, wet AMD. In some embodiments, the additional therapeutic agent is another complement-directed therapeutic agent, including another Factor D antagonist, such as another anti-Factor D antibody.

For instance, the anti-Factor D antibody formulations herein may be administered in combination with an effective amount of a VEGF antagonist, such as an anti-VEGF antibody optionally in combination with another Factor D antagonist, such as another anti-Factor D antibody. Anti-VEGF antibodies are described, for example, in U.S. Pat. No. 6,884,879 issued Feb. 26, 2015, WO98/45331; WO2005/012359; WO2005/044853; and WO98/45331. In various embodiments, anti-VEGF drugs to be administered in combination with the anti-Factor D antibody formulations herein include AVASTIN® (bevacizumab) and/or LUCENTIS® (ranibizumab), optionally in combination with at least one additional Factor D antagonist/antibody.

The anti-Factor D antibody formulations herein may also be administered in combination with an effective amount of an HTRA1 antagonist, such as, for example, an anti-HTRA1 antibody optionally in combination with at least one additional Factor D antagonist/antibody. Anti-HTRA1 antibodies are described, for example, in WO 2013055998 A1.

The anti-Factor D antibody formulations herein may also be administered in combination with an effective amount of an Angiopoietin-2 (Ang2) antagonist, such as an anti-Ang2 antibody optionally in combination with at least one additional Factor D antagonist/antibody. Anti-Ang2 antibodies are disclosed, for example, in US 20090304694 A1.

The anti-Factor D antibody formulations herein may further be administered in combination with an effective amount of an TIE2 antagonist, such as an anti-TIE2 antibody optionally in combination with at least one additional Factor D antagonist/antibody. Anti-TIE 2 antibodies are described in U.S. Pat. No. 6,376,653.

Other therapeutic agents suitable for combined administration with the anti-Factor D antibody formulations herein are antagonists of various members of the classical or alternative complement pathway (complement inhibitors). Thus, the formulations herein may be administered in combination with antagonists of one or more of the C1, C2, C3, C4, C5, C6, C7, C8, and C9 complement components. In some embodiments, the anti-Factor D formulations herein are combined with antagonists of the C2 and/or C4 and/or C5 complement components, such as anti-C2 and/or anti-C4 and/or anti-C5 antibodies. Such antibodies are known in the art and/or are commercially available. An anti-C5 antibody eculizumab (Alexion, Cheshire, Conn., USA), has been approved for the treatment of Paroxysmal nocturnal hemoglobinuria (PNH) and atypical hemolytic uremic syndrome (aHUS). Other complement inhibitors are disclosed, for example, in US Publication No. 20050036991 A1. Thus, the anti-Factor D antibody formulations herein may be administered in combination with an effective amount of one or more complement inhibitors, including, without limitation, anti-C2 and anti-C5 antibodies, optionally in combination with at least one additional Factor D antagonist/antibody.

The anti-Factor D antibodies may be administered in combination with two or more of the listed therapeutic agents, and in general, in combination with two or more therapeutic agents suitable for treatment of a complement-associated ocular disease, including an ocular disorder associated with undesirable neovascularization in the eye. Bispecific and multi-specific antibodies binding to two or more of VEGF, HTRA1, Ang2 and TIE2, or two or more complement components, are specifically included in the group of therapeutic agents that can be used in combination with the anti-Factor D formulations of the present invention, optionally in combination with another anti-Factor D antagonist/antibody.

Combined administration herein includes co-administration, using separate formulations or a single pharmaceutical formulation, and consecutive administration in either order, wherein generally there is a time period while both (or all) active agents simultaneously exert their biological activities.

These second medicaments are generally used in the same dosages and with administration routes as used hereinbefore or about from 1 to 99% of the heretofore employed dosages. If such second medicaments are used at all, preferably, they are used in lower amounts than if the anti-factor D antibody or antigen-binding fragment thereof were not present, especially in subsequent dosings beyond the initial dosing with antibody, so as to eliminate or reduce side effects caused thereby.

Where a second medicament is administered in an effective amount with an antibody exposure, it may be administered with any exposure, for example, only with one exposure, or with more than one exposure. In some embodiments, the second medicament is administered with the initial exposure. In some embodiments, the second medicament is administered with the initial and second exposures. In some embodiments, the second medicament is administered with all exposures.

The combined administration includes co-administration (concurrent administration), using separate formulations or a single pharmaceutical formulation, and consecutive administration in either order, wherein preferably there is a time period while both (or all) active agents simultaneously exert their biological activities. In some embodiments, after the initial exposure, the amount of such agent is reduced or eliminated so as to reduce the exposure of the subject to an agent with side effects such as prednisone and cyclophosphamide, especially when the agent is a corticosteroid. In some embodiments, the amount of the second medicament is not reduced or eliminated.

Further details of the invention are illustrated by the following non-limiting examples. The following examples are offered by way of illustration and not by way of limitation. Commercially available reagents referred to in the examples were used according to manufacturer's instructions unless otherwise indicated.

In the following examples, the terms Drug Substance (DP) and Drug Product (DP) are defined as follows:

Drug Substance (DS): refers to frozen or liquid-state formulation containing the active pharmaceutical ingredient, prior to filling and lyophilization.

Drug Product (DP, lyophilized): refers to the lyophilized, solid-state formulation containing the active pharmaceutical ingredient in a vial or other container.

Drug Product (DP, reconstituted): refers to liquid-state formulation containing the active pharmaceutical ingredient after diluent is added to the vial or other container.

Drug Product (DP, without qualifying terms): refers to the lyophilized solid-state formulation containing the active pharmaceutical ingredient in a vial or other container.

In the following examples DP is 4× concentrated relative to the DS.

### Example 1

Materials and Methods

All Drug Substance (DS) and Drug Product (DP) formulations used for the studies in the following examples were dialyzed into their respective diafiltration buffers (containing no sugar or surfactant) using a Millipore Labscale™ TFF System equipped with Millipore 10 kDa Pellicon XL 50 Ultrafiltration Cassettes (Cat # PXC010C50). Sugar and surfactant were added to each formulation via dilution with conditioning buffer.

A. NaCl Concentration Determination

A solubility study was conducted in order to determine the appropriate level of NaCl to ensure robust lampalizumab solubility in the DP with an acceptable DP pH range of 5.0-5.5, protein concentration range of 90-110 mg/mL, and HisCl concentration of 40 mM. Lampalizumab was first exhaustively dialyzed into 30 mM HisCl at pH 5.6. Lampalizumab is not completely soluble in this solution. Water, NaCl from a 1 M stock, and 30 mM HisCl at pH 5.6, was then combined with the dialyzed lampalizumab in clear glass HPLC vials (Thermo Scientific Cat # C4010-V1) in order to generate samples with a final protein concentration of 115 mg/mL and varying NaCl concentrations up to 40 mM. The conditions chosen provide a gap between the sample conditions and the highest acceptable pH, highest acceptable protein concentration, and lowest anticipated HisCl concentration (accounting for potential Donnan Effect). Samples were held at ambient lab temperature to investigate the solubility dependence as a function of the basic charge variant levels.

B. Formulation Screening

After the appropriate target pH and NaCl levels were determined from the solubility studies, the set of formulations to be screened were determined. Formulations 1 and 3 are identical except for the type of sugar used as the cryoprotectant/lyoprotectant (sucrose and trehalose, respectively). Formulations 2 and 4 are included in the screen to investigate the aggregation rates of Formulations 1 and 3 with lower sugar concentrations and high protein concentrations, resulting in an inferior sugar-to-protein ratio. Formulation 5 is included to investigate DP stability with sodium chloride removed from the formulation and the histidine chloride concentration is increased to ensure lampalizumab solubility in the DP is equivalent to Formulation 1. Sodium chloride is known to decrease the collapse temperature of lyophilized cakes. Formulation 6 is therefore included to investigate the impact of higher NaCl levels on the physical stability of the cake during lyophilization and storage. The sugar concentration in Formulations 1, 3, 5, and 6 were chosen such that the target DP osmolality was approximately 330 mOsm/kg. Formulation 7 was included as a study control.

DS samples were filled in 1 mL aliquots into autoclaved 2 cc glass vials, stoppered with 13 mm liquid stoppers, and capped with 13 mm aluminum flip-top caps. DP samples were filled in 2 mL aliquots into autoclaved 6 cc glass vials and partially stoppered with 20 mm lyophilization stoppers prior to lyophilization. After lyophilization the vials were capped with 20 mm aluminum flip-top caps. All DP formulations contained 0.6-0.8% (w/w) moisture following lyophilization. DP formulations were reconstituted with purified water to a final volume of 500 μL such that the concentration of lampalizumab and all excipients was four times greater than in the DS prior to lyophilization. The reconstitution volume varied for each sample from 440-452 μL depending on the formulation composition.

C. Assays

Color, Appearance, and Clarity

Sample appearance was visually assessed against purified water using a light inspection station equipped with a white fluorescent light. The appearance of the DP was assessed prior to and after reconstitution.

Turbidity

Turbidity (forward scattering) was assessed by averaging the UV absorbance at 340, 345, 350, 355, and 360 nm. Samples were analyzed neat in a 1 cm path length quartz cuvette using an Agilent HP8453 spectrophotometer blanked with water.

PH

The solution pH was measured using a Mettler-Toledo Seven Multi pH meter standardized with pH=4.00 and 7.00 solutions.

Protein Concentration Via UV Scan (Gravimetric Dilution)

Lampalizumab concentration was determined via UV Scan using a HP8453 UV Spectrophotometer. Samples were diluted gravimetrically to approximately 0.5 mg/mL using lampalizumab DS formulation buffer. Absorption was measured in a quartz cuvette with a path length of 1 cm. The instrument was blanked with DS formulation buffer. Protein concentration was calculated using absorbance at 278 nm (A₂₇₈), absorbance at 320 nm (A₃₂₀), dilution factor (D), and an extinction coefficient, ε, of 1.39 (mg/mL)⁻¹ cm⁻¹ according to the following equation:

$$Concentration ( \frac{mg}{mL} ) = \frac{( {A}_{278} - {A}_{320} ) \times D}{ɛ \times cell path length ( cm )}$$

The dilution factor is calculated according to the following equation, where m is mass:

D=(1.05 g/mL×m_{diluted sample})/(1.01 g/mL×mₛₐₘₚₗₑ)

Protein concentration was determined using duplicate dilutions and absorbance measurements of each sample.

Molecular Size Distribution Via Size Exclusion Chromatography (SEC-HPLC)

The molecular size distribution of lampalizumab samples was determined by separating size variants on a TosoHaas TSK G2000SWXL (7.8 mm×300 mm) size exclusion column using an Agilent 1200 High Pressure Liquid Chromatography (HPLC) system equipped with UV detection at 280 nm. Samples were diluted in mobile phase (0.2M potassium phosphate, 0.25M potassium chloride, pH 6.2) to a concentration of approximately 2 mg/mL and stored at 2-8° C. until injection. Sample injections of 35 μL were analyzed at ambient temperature using a flow rate of 0.7 mL/min. Lampalizumab Lot FCD508-1 was injected as a reference material and DS formulation buffer was used for reagent blanks. Peak areas were integrated with respect to the baseline. Duplicate sample injections were used to determine the molecular size distribution.

Charge Heterogeneity Via Ion Exchange Chromatography (IEC)

The charge heterogeneity of lampalizumab samples was determined by separating charge variants on Thermo Fisher Scientific ProPac® SAX-10, 4×250 mm strong anion exchange column using an Agilent 1200 High Pressure Liquid Chromatography (HPLC) system with UV detection at 280 nm. Samples were diluted to 2 mg/mL with 20 mM 2-Amino-2-methyl-1,3-propanediol (AMPD) at pH 8.2 and buffer exchanged into 20 mM AMPD using NAP™ 5 columns and stored at 2-8° C. until injection. Sample injections of 50 μL were separated on the column at a flow rate of 0.8 mL/min at 40° C. using a linear gradient of 25 mM to 200 mM NaCl in AMPD at pH 8.2 over 50 minutes. The column was then washed with 500 mM NaCl in AMPD at pH 8.2 for 10 minutes. Lot FCD508-1 was injected as a reference material and DS formulation buffer was used for reagent blanks.

Capillary Electrophoresis-Sodium Dodecyl Sulfate (Non-Reduced) (CE-SDS)

The purity of non-reduced lampalizumab samples was determined using a capillary electrophoresis (CE) Beckman PA800 plus system with LIF detection. Separation was obtained by applying a 15 kV voltage differential across a 31 cm capillary (10 cm to detector) over a run time of 16 minutes. The capillary temperature was maintained at 20° C. Samples were denatured with sodium dodecyl sulfate (SDS) and fluorescently labeled with 3-(2-furoyl)quinolone-2-carboxaldehyde (FQ dye). Lampalizumab was injected as a reference material and DS formulation buffer was used for reagent blanks. Peak areas were integrated with respect to the baseline and the value for peak area was divided by migration time to give a corrected peak area (CPA). Only Formulation 1 samples were monitored by NR CE-SDS at select time points to reduce the sample load.

Binding by ELISA

The wells of a high binding polystyrene microtiter plate are coated with Factor D, washed, exposed to varying concentrations of lampalizumab in formulation buffer, and washed. The plates are then exposed to goat Anti-F(abI-HRP antibodies and washed. SureBlue Reserve solution is then added to each well and incubated prior to the addition of 0.6 N sulfuric acid. Optical density values of each well are then measured at 450 nm (650 nm reference absorbance) to determine the lampalizumab concentration in each well.

Subvisible Particles by Light Obscuration

A HIAC 9703 particle counter was used to count the number of subvisible particulates of sizes greater than or equal to 2, 5, 10, 25, and 50 μm. A total of four injections of 0.4 mL each were performed per sample. Reported particle counts indicate the average of the final three runs (the first run was discarded).

Moisture

The volumetric Karl Fischer moisture assay was performed as follows. The cake from a single DP vial was crushed and placed into 15 mL sample tube and analyzed using Mitsubishi Model RV 2AJ-511 TIX robotic titration system filled with Hydranal® Composite 2 volumetric Karl Fischer reagent. The instrument is standardized with sodium tartate dehydrate prior to sample analysis.

Osmolality

Osmolality of the lampalizumab samples was determined by freezing point depression in triplicate using an Advanced Instruments 3300 osmometer.

### Example 2

Formulation Studies

The lampalizumab formulations contained the following ingredients: histidine hydrochloride monohydrate, histidine free base, sodium chloride, sucrose, trehalose dihydrate and Polysorbate 20. The list of Drug Substance (DS) formulations screened is set forth in Table 1.

Results

A. Stabilizer Concentration Determination

To verify that the solubility of lampalizumab was a function of basic charge variant levels, fresh and stressed lampalizumab were simultaneously dialyzed into 30 mM HisCl and 12 mM NaCl at pH 5.6 to a final concentration of 115 mg/mL. The stressed lampalizumab was generated by titrating the fresh material to pH 5.5 with 0.1 N HCl and incubating it at 50° C. for 18 hours before incubating it at 40° C. for 18 hours. This resulting sample contained 27% basic charge variants by IEC.

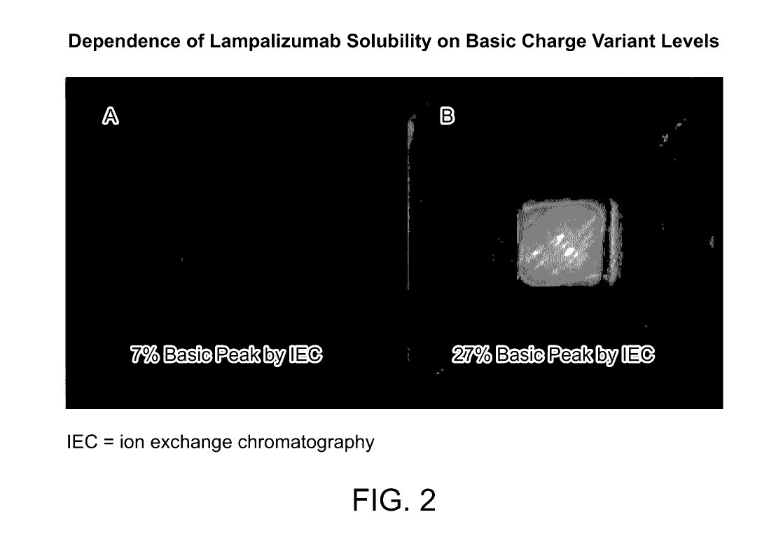

FIG. 2 shows that after dialysis, the fresh material is fully soluble (clear solution with no turbidity) at ambient temperature but the stressed material is not (white solution with turbidity).

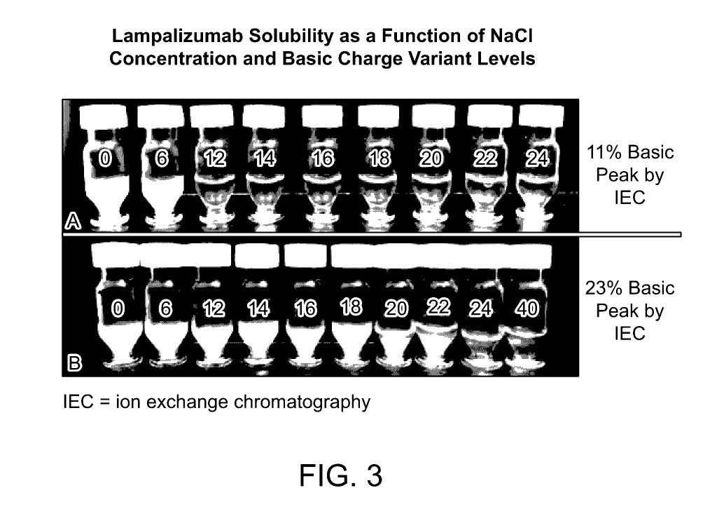

FIG. 3 shows that 12 mM of NaCl is required to maintain solubility (clear solution with no turbidity) when lampalizumab containing 11% basic charge variants is formulated at 115 mg/mL in 30 mM HisCl at pH 5.6. However, 24 mM of NaCl is required to maintain solubility when the samples were stored at room temperature for 23 days until the basic charge variant levels were at 23%. No change in acidic charge variants was observed during storage at ambient temperature. This NaCl concentration also allows for sufficiently low sub-visible particle levels to meet the USP<789> criteria via light obscuration.

B. Formulation Screening

1. DS

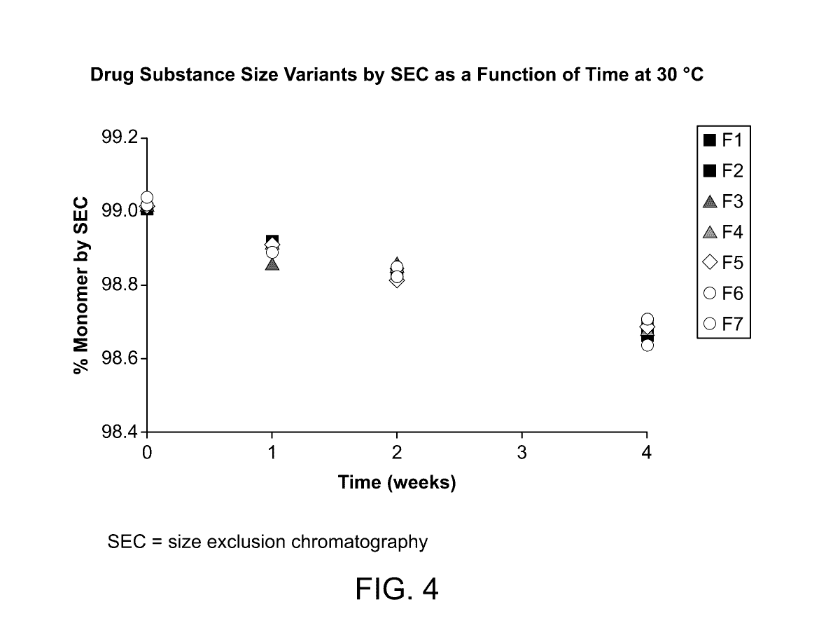

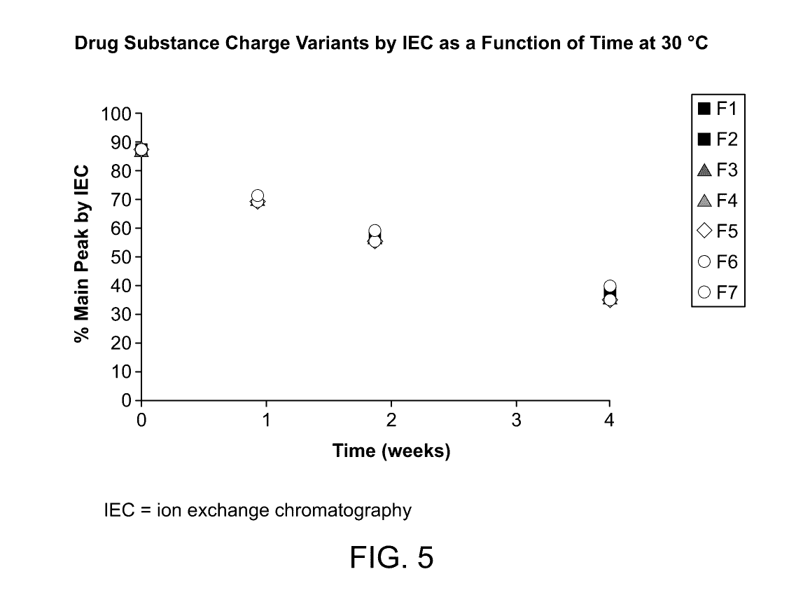

The raw data for the DS formulations stored under real-time, accelerated, and stress conditions in vials are shown in Tables 2, 3, and 4, respectively. During storage at 30° C. (stress conditions) for up to four weeks, no difference in the rate of size variant or charge variant formation was observed between the DS formulations (FIG. 4 and FIG. 5 respectively). Assuming zero-order kinetics, the rate of main peak loss by IEC varied from 12.4-12.9%/week for Formulations 1-6. The potency of Formulation 7 DS was reduced from 98% to 87% binding (Q12713) after storage at 30° C. for four weeks.

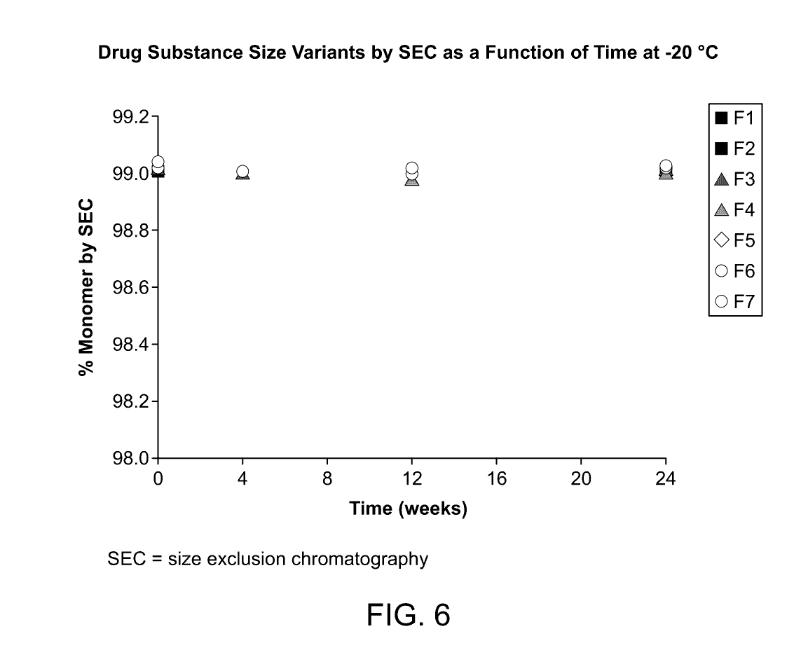

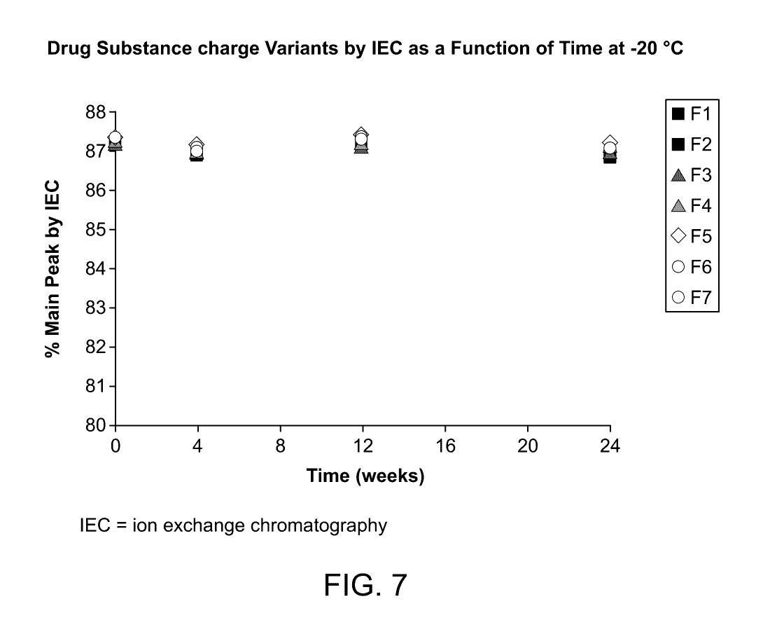

No difference in the rate of size variant or charge variant formation was observed between DS formulations during storage at 5° C. (accelerated conditions) for up to eight weeks. No changes in the level of size variants by SEC or charge variants were observed in any formulation during DS storage at −20° C. for up to 24 weeks (FIG. 6 and FIG. 7, respectively). No change in size variant levels was observed by NR CE-SDS in Formulation 1 DS after storage at −20° C. for 12 weeks (data not shown). All DS formulations were found to contain histidine concentrations within 8% of their target value as determined by free amino acid analysis.

2. DP

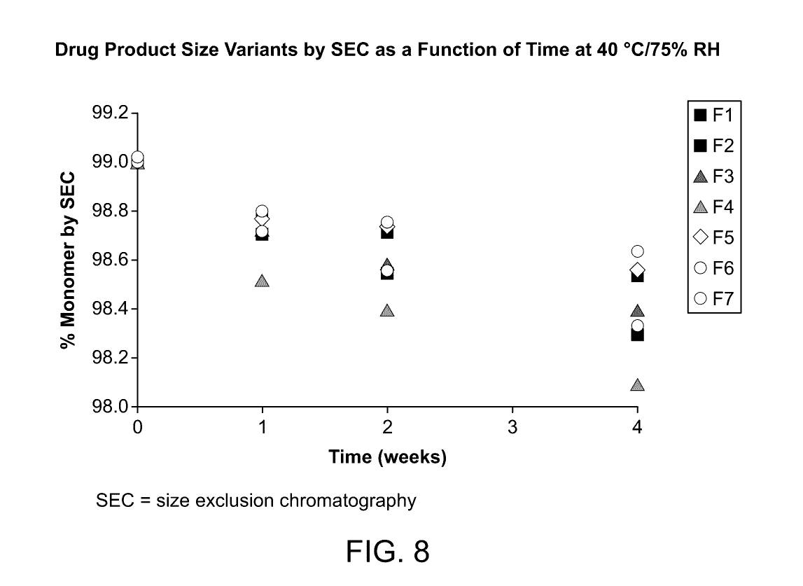

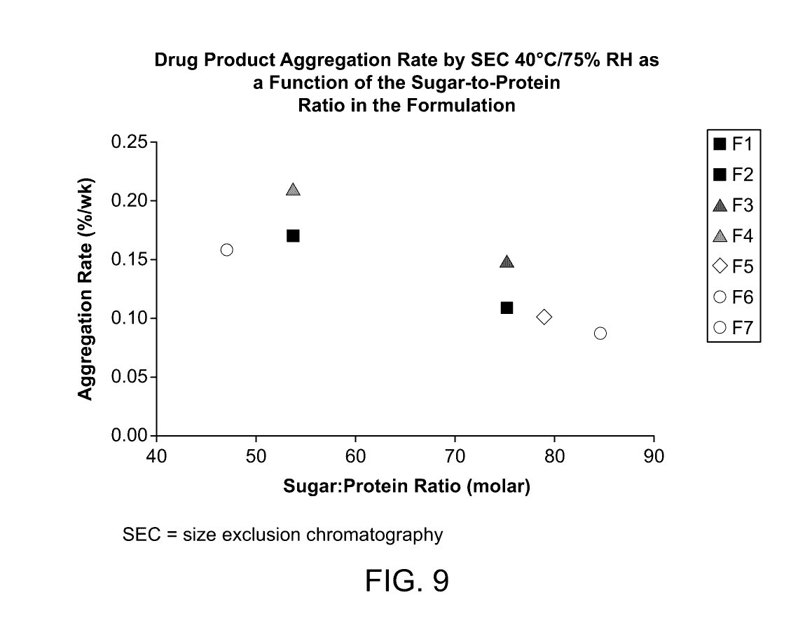

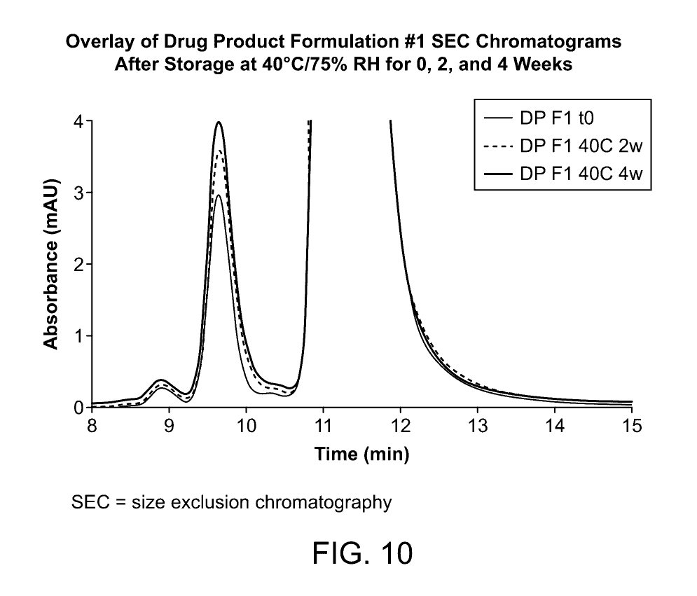

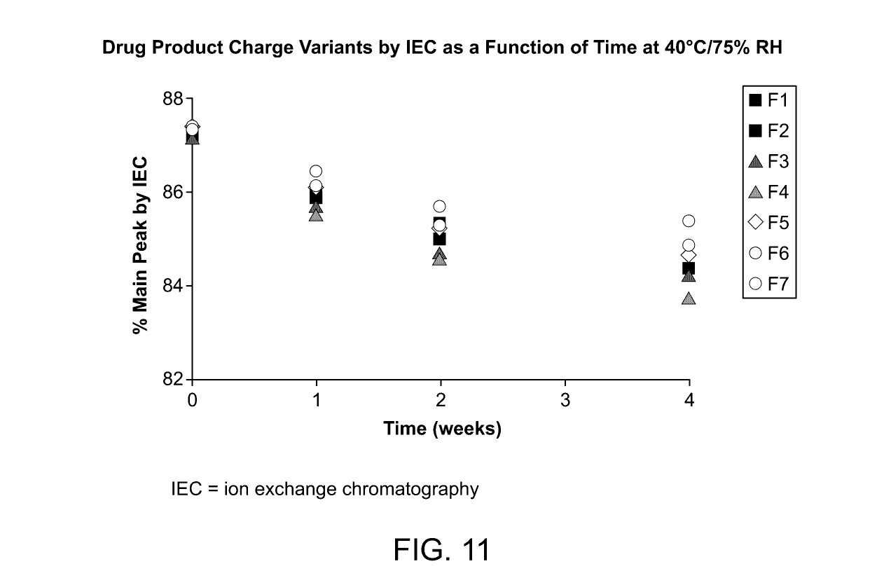

The raw data for the DP formulations stored under real-time, accelerated, and stress conditions are shown in Tables 5A and 5B, 6A and 6B, and 7A and 7B, respectively. The osmolality of Formulations 1, 3, 5, and 6 at time zero were all 330±10 mOsm/kg, as expected. The change in size variants during DP storage at 40° C./75% RH (stress conditions) for up to four weeks is shown in FIG. 8. The increase in size variant formation in Formulations 3 and 4 was greater than in Formulations 1 and 2, respectively. This indicates that sucrose limits lampalizumab aggregation in the DP better than trehalose under stress conditions. FIG. 9 shows that the aggregation rates at 40° C./75% RH correlate negatively with the sugar-to-protein ratio in the DP. An overlay of Formulation 1 SEC chromatograms at time zero and after storage at 40° C./75% RH for two and four weeks is shown in FIG. 10. The primary size variant that formed in the DP under stress conditions was a dimer species; minimal higher molecular weight species were formed under stress conditions up to four weeks. The change in charge variant levels during DP storage at 40° C./75% RH for up to four weeks is shown in FIG. 11. No clear trend in the rate of charge variant formation was observed between formulations. However, the sucrose-based formulations appear to have lower levels of charge variants than the trehalose-based formulations.

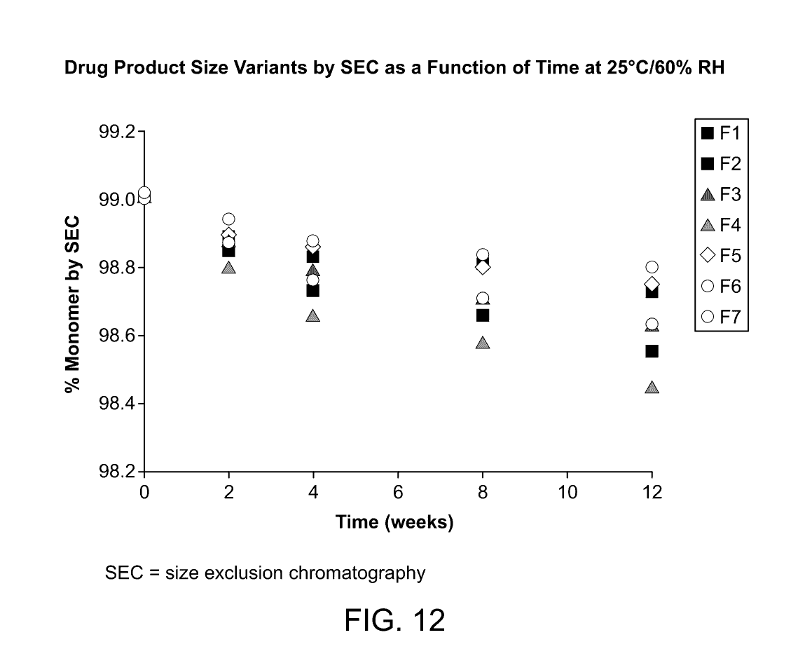

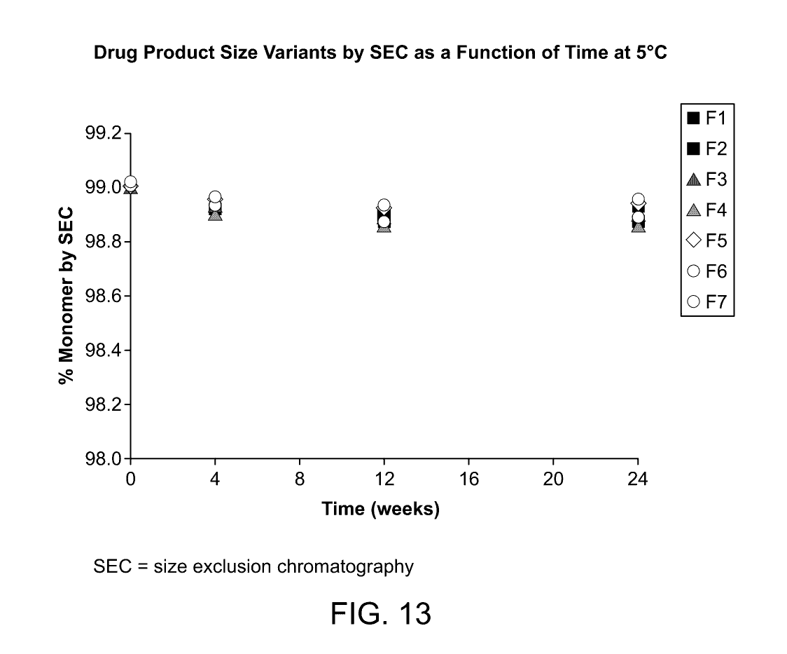

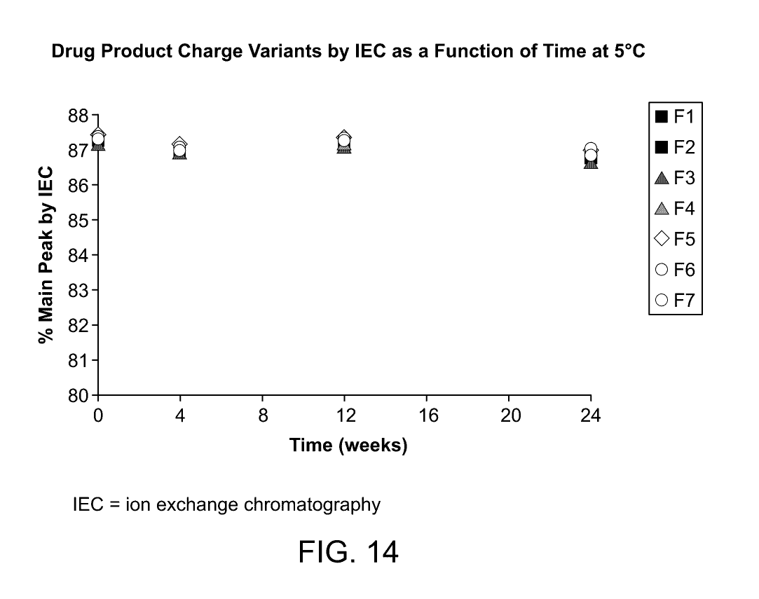

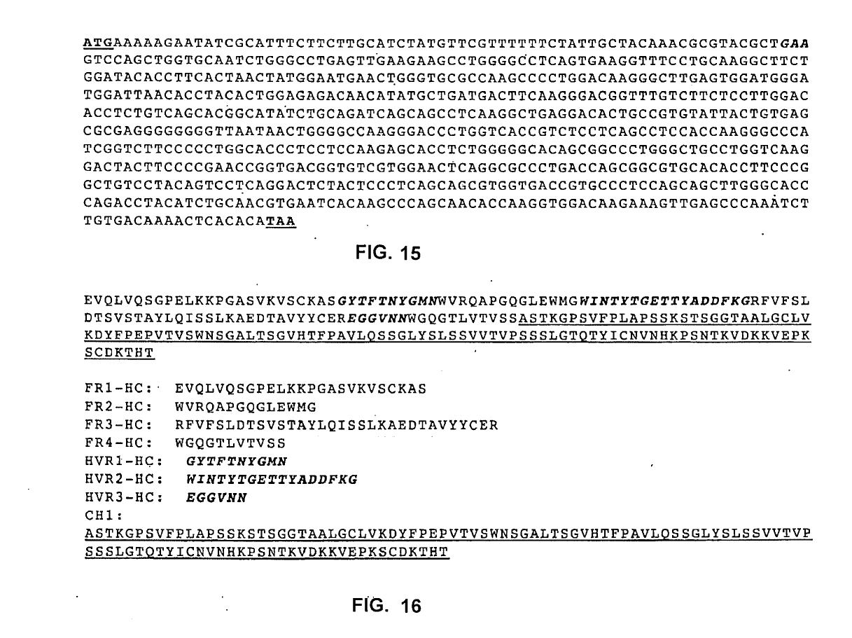

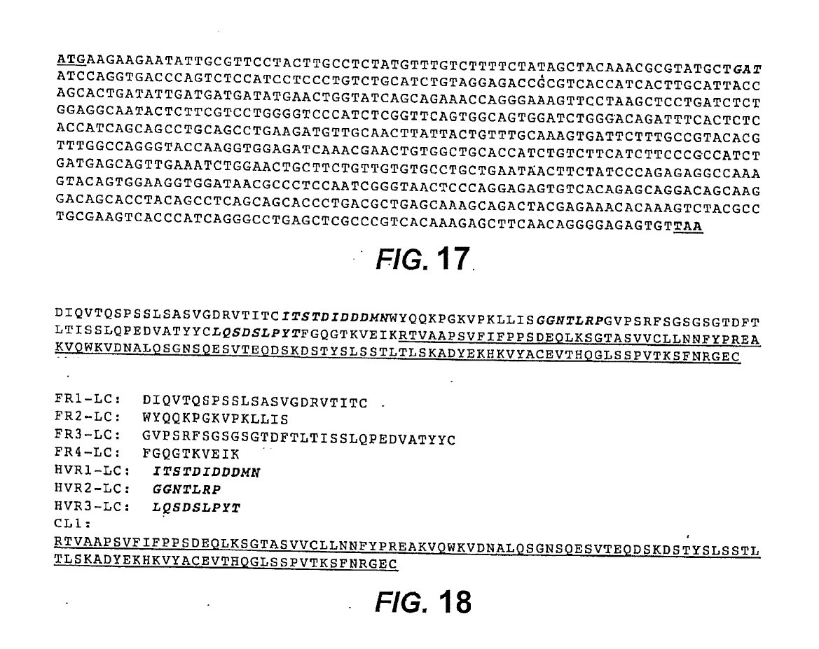

The change in size variants during DP storage at 25° C./60% RH (accelerated conditions) for up to 12 weeks is shown in FIG. 12. The aggregation rates at 25° C./60% RH correlate well with the aggregation rates at 40° C./75% RH and further demonstrate that trehalose is inferior to sucrose at limiting lampalizumab aggregation during DP storage at elevated temperatures. No difference in the rate of charge variant formation was observed between all formulations during DP storage at 25° C./60% RH (accelerated conditions) for up to 12 weeks (data not shown). No change in size variants by SEC or charge variant levels was observed in the DP during storage at 5° C. for up to 24 weeks (FIG. 13 and FIG. 14, respectively). No change in size variant levels was observed by NR CE-SDS in Formulation 1 DP after storage at 5° C. for 12 weeks (data not shown).

Discussion

A. NaCl Concentration Determination

FIG. 2 shows that the solubility of lampalizumab in a given solution is reduced as the level of basic charge variants increases. It is therefore important to control basic charge variant levels when determining the NaCl concentration needed to ensure lampalizumab solubility. The solubility study shown in FIG. 3 supports a DP pH up to 5.5 at a protein concentration of 100±10 mg/mL, NaCl concentration of 28±4 mM, and HisCl concentration of 40±10 mM to allow for manufacturing variability in the DS and ensure robust solubility of lampalizumab containing up to 22% basic charge variants.

B. Formulation Screening

No differences in the size or charge variant formation rates were detected between the candidate DS formulations after storage at −20° C. for six months, 5° C. for eight weeks, or 30° C. for four weeks. Formulation selection was therefore based upon assessment of the stability of the DP formulations.

Lyoprotectant Selection

Based on the aggregation rate of DP Formulations 3 and 4 relative to Formulations 1 and 2, respectively, it appears that trehalose is not as effective as sucrose at minimizing lampalizumab aggregation under accelerated and stress conditions (FIGS. 8 and 12). Additionally, the level of charge variants increased faster in the DP formulations containing trehalose than in the equivalent formulations containing sucrose under stress conditions (FIG. 11). Therefore, sucrose was chosen as the lyoprotectant species for the lampalizumab formulation. Although lyophilized protein/trehalose systems have higher glass transition temperatures than protein/sucrose systems at low water content (Duddu et al. (1997). The Relationship Between Protein Aggregation and Molecular Mobility Below the Glass Transition Temperature of Lyophilized Formulations Containing a Monoclonal Antibody. Pharm Research 14(5):596-600; Pikal M J et al. (2008). Solid State Chemistry of Proteins: II. The Correlation of Storage Stability of Freeze-Dried Human Growth Hormone (hGH) with Structure and Dynamics in the Glassy Solid. J Pharm Sci: 97(12):5106-21), sucrose has been previously shown to be a superior lyoprotectant for reducing aggregation and chemical degradation rates (Pikal et al., supra). Pikal et al. suggest that chemical degradation and aggregation rates may correlate with fast dynamic time constants as measure by neutron scattering rather than the difference between the storage temperature and the glass transition temperature of the solid formulation, and sucrose shows greater suppression of fast dynamics than trehalose (Pikal et al., supra). The moisture level of all DP formulations were between 0.6 and 0.8% w/w at time zero, so it is unlikely that residual moisture can account for the difference in degradation rates between the sucrose- and trehalose-based formulations.

Formulation Selection

As expected, the rate of aggregation in the DP correlated negatively with the sugar-to-protein ratio under stress conditions (FIG. 9). This indicates that it is ideal to maximize the amount of sucrose in the formulation. Formulations 1 and 5 had the highest sucrose levels of the six formulations screened (not counting the control). No difference in DS or DP stability was observed between Formulation 1 and Formulation 5 under all storage conditions investigated. However, at the time of formulation selection, there was no known clinical experience with intravitreal administration of solutions containing greater than 40 mM of HisCl. Formulation 5 contains 64 mM of HisCl and therefore presents additional clinical risk by increasing the buffering capacity of the DP. The equilibrated pH of the vitreous would therefore be lower upon administration of Formulation 5 relative to the other formulations. Formulation 1 is therefore preferable to Formulation 5 because it presents a lower clinical risk. Additionally, no cake collapse or excessive instability was observed in Formulation 6, which contained 15 mM of NaCl. This indicates that the 7 mM of NaCl in Formulation 1 is not likely to result in any lyo cake collapse or macroscopic physical instability.

The Drug Substance (pre-lyophilized) and Drug Product (lyophilized) formulated with the selected formulation is stable for up to two years under recommended storage conditions, −20° C. for Drug Substance and 5° C. for Drug Product, as attested by the stability data set forth in Tables 9, 10A and 10B.

1. A pharmaceutical formulation comprising a therapeutically effective amount of a monoclonal anti-Factor D antibody, a buffer adjusting the pH to between 5.0 and 5.4, a lyoprotectant and a surfactant.

2. The pharmaceutical formulation of claim 1, wherein the pH is about 5.3.

3. The pharmaceutical formulation of claim 1, wherein the lyoprotectant to antibody ratio is about 60 to 100 mole lyoprotectant:1 mole antibody.

4. The pharmaceutical formulation of claim 3, wherein the lyoprotectant to antibody ratio is about 80 mole lyoprotectant:1 mole antibody.

5. The pharmaceutical formulation of claim 1, wherein the buffer is a histidine buffer.

6. The pharmaceutical formulation of claim 5, wherein the histidine buffer is present in an amount of about 5 mM to about 15 mM.

7. The pharmaceutical formulation of claim 6, wherein the histidine buffer is present in an amount of about 7 mM to about 13 mM.

8. The pharmaceutical formulation of claim 1, wherein the lyoprotectant comprises one or more polyols.

9. The pharmaceutical formulation of claim 8, wherein at least one of the polyols is a reducing or a non-reducing sugar selected from the group consisting of α,α-trehalose and sucrose.

10.-11. (canceled)

12. The pharmaceutical formulation of claim 8, wherein at least one of the polyols is a disaccharide.

13. The pharmaceutical formulation of claim 1, wherein the surfactant comprises one or more polysorbates and/or poloxamers.

14. (canceled)

15. The pharmaceutical formulation of claim 1, wherein said monoclonal anti-Factor D antibody comprises heavy chain hypervariable regions (HVR-HCs) having at least 98% or at least 99% sequence identity to the HVR sequences of HVR1-HC: GYTFTNYGMN (SEQ ID NO: 3); HVR2-HC: WINTYTGETTYADDFKG (SEQ ID NO: 4); HVR3-HC: EGGVNN (SEQ ID NO: 5) and/or light chain hypervariable regions (HVR-LCs) having at least 98% or at least 99% sequence identity to the HVR-LC sequences of HVR1-LC: ITSTDIDDDMN (SEQ ID NO: 8); HVR2-LC: GGNTLRP (SEQ ID NO: 9); and HVR3-LC: LQSDSLPYT (SEQ ID NO: 10).

16. The pharmaceutical formulation of claim 15, wherein said monoclonal anti-Factor D antibody comprises the HVR-HCs of HVR1-HC: GYTFTNYGMN (SEQ ID NO: 3); HVR2-HC: WINTYTGETTYADDFKG (SEQ ID NO: 4); HVR3-HC: EGGVNN (SEQ ID NO: 5) and/or the HVR-LC of HVR1-LC: ITSTDIDDDMN (SEQ ID NO: 8); HVR2-LC: GGNTLRP (SEQ ID NO: 9); and HVR3-LC: LQSDSLPYT (SEQ ID NO: 10).

17. The pharmaceutical formulation of claim 15, wherein said monoclonal anti-Factor D antibody comprises a heavy chain variable region sequence having at least 85%, or at least 90%, or at least 95%, or at least 98%, or at least 99% sequence identity to the variable region sequence of the heavy chain of SEQ ID NO: 2 and/or a light chain variable region sequence having at least 85%, or at least 90%, or at least 95%, or at least 98%, or at least 99% sequence identity to the variable region sequence of the light chain of SEQ ID NO: 7.

18. The pharmaceutical formulation of claim 17, wherein said monoclonal anti-Factor D antibody comprises the variable region sequence of the heavy chain of SEQ ID NO: 2 and/or the variable region sequence of the light chain of SEQ ID NO: 7.

19. The pharmaceutical formulation of claim 18, wherein said monoclonal anti-Factor D antibody comprises a heavy chain sequence comprising SEQ ID NO: 2 and/or a light chain sequence comprising SEQ ID NO: 7.

20.-21. (canceled)

22. The pharmaceutical formulation of claim 1, wherein said monoclonal anti-Factor D antibody is an antibody fragment.

23. (canceled)

24. The pharmaceutical formulation of claim 1, wherein said monoclonal anti-Factor D antibody is humanized.

25. The pharmaceutical formulation of claim 1, wherein said monoclonal anti-Factor D antibody is lampalizumab.

26. The pharmaceutical formulation of claim 1, which is stable upon freezing and thawing.

27. The pharmaceutical formulation of claim 1, which is a pre-lyophilized formulation.

28. The pharmaceutical formulation of claim 27, which is stable at −20° C. storage temperature for at least one year.

29. (canceled)

30. The pharmaceutical formulation of claim 1, which is lyophilized.

31. The pharmaceutical formulation of claim 30, which is stable at 5° C. storage temperature for at least one year.

32. (canceled)

33. The pharmaceutical formulation of claim 1, which is a liquid formulation.

34. The pharmaceutical formulation of claim 33, which is for intraocular administration.

35.-38. (canceled)

39. A reconstituted aqueous liquid formulation prepared from the pharmaceutical formulation of claim 1.

40. The reconstituted aqueous liquid formulation of claim 41 prepared directly by the reconstitution of the lyophilized formulation of claim 30 or 31.

41. A pre-lyophilized or lyophilized pharmaceutical formulation comprising a therapeutically effective amount of a monoclonal anti-Factor D antibody, about 5 mM to about 15 mM of a histidine buffer adjusting the pH to between 5.0 and 5.4, sodium chloride, a lyoprotectant and a surfactant.

42. The pre-lyophilized or lyophilized pharmaceutical formulation of claim 41, wherein said anti-Factor D antibody comprises heavy chain hypervariable regions (HVR-HCs) having at least 98% or at least 99% sequence identity to the HVR sequences of HVR1-HC: GYTFTNYGMN (SEQ ID NO: 3); HVR2-HC: WINTYTGETTYADDFKG (SEQ ID NO: 4); HVR3-HC: EGGVNN (SEQ ID NO: 5) and/or light chain hypervariable regions (HVR-LCs) having at least 98% or at least 99% sequence identity to the HVR-LC sequences of HVR1-LC: ITSTDIDDDMN (SEQ ID NO: 8); HVR2-LC: GGNTLRP (SEQ ID NO: 9); and HVR3-LC: LQSDSLPYT (SEQ ID NO: 10).

43. The pre-lyophilized or lyophilized pharmaceutical formulation of claim 42, wherein said monoclonal anti-Factor D antibody comprises the heavy chain hypervariable regions (HVR-HCs) of HVR1-HC: GYTFTNYGMN (SEQ ID NO: 3); HVR2-HC: WINTYTGETTYADDFKG (SEQ ID NO: 4); HVR3-HC: EGGVNN (SEQ ID NO: 5) and/or the light chain hypervariable regions (HVR-LCs) of HVR1-LC: ITSTDIDDDMN (SEQ ID NO: 8); HVR2-LC: GGNTLRP (SEQ ID NO: 9); and HVR3-LC: LQSDSLPYT (SEQ ID NO: 10).

44. The pre-lyophilized or lyophilized pharmaceutical formulation of claim 42, wherein said monoclonal anti-Factor D antibody comprises a heavy chain variable region sequence having at least 85%, or at least 90%, or at least 95%, or at least 98%, or at least 99% sequence identity to the heavy chain of SEQ ID NO: 2 and/or a light chain variable region sequence having at least 85%, or at least 90%, or at least 95%, or at least 98%, or at least 99% sequence identity to the light chain of SEQ ID NO: 7.

45. The pre-lyophilized or lyophilized pharmaceutical formulation of claim 44, wherein said monoclonal anti-Factor D antibody comprises a heavy chain sequence comprising SEQ ID NO: 2 and/or a light chain sequence comprising SEQ ID NO: 7.

46.-47. (canceled)

48. The pre-lyophilized or lyophilized pharmaceutical formulation of claim 41, wherein said monoclonal anti-Factor D antibody is an antibody fragment.

49. (canceled)

50. The pre-lyophilized or lyophilized pharmaceutical formulation of claim 41, wherein said monoclonal anti-Factor D antibody is humanized.

51. The pre-lyophilized or lyophilized pharmaceutical formulation of claim 50, wherein said monoclonal anti-Factor D antibody is lampalizumab.

52. The pre-lyophilized or lyophilized pharmaceutical formulation of claim 51 comprising about 25 mg/mL of lampalizumab.

53. The pre-lyophilized or lyophilized pharmaceutical formulation of claim 41, wherein in the lyophilized formulation the lyoprotectant to antibody ratio is about 60 to 100 mole lyoprotectant:1 mole antibody.

54. The pre-lyophilized or lyophilized pharmaceutical formulation of claim 41, wherein in the lyophilized formulation the sucrose to antibody ratio is about 80 mole lyoprotectant:1 mole antibody.

55. A reconstituted aqueous liquid formulation prepared from the lyophilized pharmaceutical formulation of claim 41.

56. The reconstituted formulation of claim 55, which is for intraocular administration.

57.-58. (canceled)

59. The reconstituted formulation of claim 56, comprising about 100 mg/mL of lampalizumab.

60. An aqueous liquid pharmaceutical formulation comprising a therapeutically effective amount of a monoclonal anti-Factor D antibody, about 20 mM to about 60 mM of histidine chloride, a polyol, sodium chloride and a surfactant.

61. The liquid formulation of claim 60 wherein said anti-Factor D antibody comprises heavy chain hypervariable regions (HVR-HCs) having at least 98% or at least 99% sequence identity to the HVR sequences of HVR1-HC: GYTFTNYGMN (SEQ ID NO: 3); HVR2-HC: WINTYTGETTYADDFKG (SEQ ID NO: 4); HVR3-HC: EGGVNN (SEQ ID NO: 5) and/or light chain hypervariable regions (HVR-LCs) having at least 98% or at least 99% sequence identity to the HVR-LC sequences of HVR1-LC: ITSTDIDDDMN (SEQ ID NO: 8); HVR2-LC: GGNTLRP (SEQ ID NO: 9); and HVR3-LC: LQSDSLPYT (SEQ ID NO: 10).

62. The liquid formulation of claim 61, wherein said monoclonal anti-Factor D antibody comprises the heavy chain hypervariable regions (HVR-HCs) of HVR1-HC: GYTFTNYGMN (SEQ ID NO: 3); HVR2-HC: WINTYTGETTYADDFKG (SEQ ID NO: 4); HVR3-HC: EGGVNN (SEQ ID NO: 5) and/or the light chain hypervariable regions (HVR-LCs) of HVR-LC sequences of HVR1-LC: ITSTDIDDDMN (SEQ ID NO: 8); HVR2-LC: GGNTLRP (SEQ ID NO: 9); and HVR3-LC: LQSDSLPYT (SEQ ID NO: 10).

63. The liquid formulation of claim 60, wherein said monoclonal anti-Factor D antibody comprises a heavy chain variable region sequence having at least 85%, or at least 90%, or at least 95%, or at least 98%, or at least 99% sequence identity to the heavy chain of SEQ ID NO: 2 and/or a light chain variable region sequence having at least 85%, or at least 90%, or at least 95%, or at least 98%, or at least 99% sequence identity to the light chain of SEQ ID NO: 7.

64. The liquid formulation of claim 63, wherein said monoclonal anti-Factor D antibody comprises a heavy chain sequence comprising SEQ ID NO: 2 and/or a light chain sequence comprising SEQ ID NO: 7.

65.-66. (canceled)

67. The liquid formulation of claim 60, wherein said monoclonal anti-Factor D antibody is an antibody fragment.

68. (canceled)

69. The liquid formulation of claim 60, wherein said monoclonal anti-Factor D antibody is humanized.

70. The liquid formulation of claim 69, wherein said anti-Factor D antibody is lampalizumab.

71. The liquid formulation of claim 60, which is for intravitreal intraocular administration.

72. (canceled)

73. The liquid formulation of claim 5, comprising about 100 mg/mL lampalizumab.

74. The liquid formulation of claim 60, which has an ionic strength equivalent to about 37 to 88 mM sodium chloride.

75. The liquid formulation of claim 74, which has an ionic strength equivalent to about 63 mM sodium chloride.

76. The liquid formulation of claim 60, which is a reconstituted liquid formulation.

77. A lyophilized formulation comprising a monoclonal anti-Factor D antibody, wherein said lyophilized formulation upon reconstitution yields an aqueous liquid formulation comprising a therapeutically effective amount of said anti-Factor D antibody, about 20 mM to about 60 mM of histidine chloride, a polyol, sodium chloride and a surfactant.

78. The lyophilized formulation of claim 77, wherein in the lyophilized formulation the polyol to antibody ratio is about 80 mole polyol:1 mole antibody.

79. (canceled)

80. The lyophilized formulation of claim 77, wherein said anti-Factor D antibody comprises heavy chain hypervariable regions (HVRs) having at least 98% or at least 99% sequence identity to the HVR sequences of HVR1-HC: GYTFTNYGMN (SEQ ID NO: 3); HVR2-HC: WINTYTGETTYADDFKG (SEQ ID NO: 4); HVR3-HC: EGGVNN (SEQ ID NO: 5) and/or light chain hypervariable regions (HVR-LCs) having at least 98% or at least 99% sequence identity to the HVR-LC sequences of HVR1-LC: ITSTDIDDDMN (SEQ ID NO: 8); HVR2-LC: GGNTLRP (SEQ ID NO: 9); and HVR3-LC: LQSDSLPYT (SEQ ID NO: 10).

81. The lyophilized formulation of claim 80, wherein said monoclonal anti-Factor D antibody comprises the heavy chain hypervariable regions (HVR-HCs) of HVR1-HC: GYTFTNYGMN (SEQ ID NO: 3); HVR2-HC: WINTYTGETTYADDFKG (SEQ ID NO: 4); HVR3-HC: EGGVNN (SEQ ID NO: 5) and/or the light chain hypervariable regions (HVR-LCs) of HVR1-LC: ITSTDIDDDMN (SEQ ID NO: 8); HVR2-LC: GGNTLRP (SEQ ID NO: 9); and HVR3-LC: LQSDSLPYT (SEQ ID NO: 10).

82. The lyophilized formulation of claim 81, wherein said monoclonal anti-Factor D antibody comprises a heavy chain variable region sequence having at least 85%, or at least 90%, or at least 95%, or at least 98%, or at least 99% sequence identity to the heavy chain of SEQ ID NO: 2 and/or a light chain variable region sequence having at least 85%, or at least 90%, or at least 95%, or at least 98%, or at least 99% sequence identity to the light chain of SEQ ID NO: 7.

83. The lyophilized formulation of claim 82, wherein said monoclonal anti-Factor D antibody comprises a heavy chain sequence comprising SEQ ID NO: 2 and/or a light chain sequence comprising SEQ ID NO: 7.

84.-85. (canceled)

86. The lyophilized formulation of claim 77, wherein said monoclonal anti-Factor D antibody is an antibody fragment.

87. (canceled)

88. The lyophilized formulation of claim 77, wherein said monoclonal anti-Factor D antibody is humanized.

89. The lyophilized formulation of claim 88, wherein said anti-Factor D antibody is lampalizumab.

90. The lyophilized formulation of claim 89, wherein the aqueous liquid formulation yielded by reconstitution is for intravitreal intraocular administration.

91. (canceled)

92. The lyophilized formulation of claim 90, wherein the aqueous liquid formulation yielded by reconstitution comprises about 100 mg/mL lampalizumab.

93. The lyophilized formulation of claim 89, wherein the aqueous liquid formulation yielded by reconstitution has an ionic strength equivalent to about 37 to 88 mM sodium chloride.

94. The lyophilized formulation of claim 93, wherein the aqueous liquid formulation yielded by reconstitution has an ionic strength equivalent to about 63 mM sodium chloride.

95. The lyophilized formulation of claim 77, which is stable at 5° C. storage temperature for at least one year.

96. (canceled)

97. A syringe for intravitreal injection comprising the reconstituted formulation of any one of claims 34, 56, 71, and 90.

98. A method of making a pharmaceutical formulation comprising:
(a) preparing the formulation of claims 41, 60, and 77; and
(b) evaluating physical stability, chemical stability, or biological activity of the monoclonal anti-Factor D antibody in the formulation.

99. A method for treatment of a complement-associated ocular disease comprising administering to a subject in need a reconstituted formulation of any one of claims 34, 56, 71, and 90.

100. The method of claim 99, wherein the complement-associated ocular disease is selected from the group consisting of age-related macular degeneration (AMD), diabetic retinopathy, choroidal neovascularization (CNV), uveitis, diabetic macular edema, pathological myopia, von Hippel-Lindau disease, histoplasmosis of the eye, Central Retinal Vein Occlusion (CRVO), corneal neovascularization, and retinal neovascularization.

101. The method of claim 100, wherein said AMD is dry AMD.

102. (canceled)

103. The method of claim 100, wherein the formulation is administered by intravitreal injection.

104.-108. (canceled)


SEQUENCE LISTING (101 sequences)

SEQ ID NO: 1 - DNA, 741 bp, Artificial Sequence
  Description of Artificial Sequence Synthetic polynucleotide
atgaaaaagaatatcgcatttcttcttgcatctatgttcgttttttctattgctacaaacgcgtacgctgaagtccagctggtgcaatctgggcctgagttgaagaagcctggggcctcagtgaaggtttcctgcaaggcttctggatacaccttcactaactatggaatgaactgggtgcgccaagcccctggacaagggcttgagtggatgggatggattaacacctacactggagagacaacatatgctgatgacttcaagggacggtttgtcttctccttggacacctctgtcagcacggcatatctgcagatcagcagcctcaaggctgaggacactgccgtgtattactgtgagcgcgaggggggggttaataactggggccaagggaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacataa

SEQ ID NO: 2 - protein, 223 aa, Artificial Sequence
  Description of Artificial Sequence Synthetic polypeptide
EVQLVQSGPELKKPGASVKVSCKASGYTFTNYGMNWVRQAPGQGLEWMGWINTYTGETTYADDFKGRFVFSLDTSVSTAYLQISSLKAEDTAVYYCEREGGVNNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 3 - protein, 10 aa, Artificial Sequence
  Description of Artificial Sequence Synthetic peptide
GYTFTNYGMN

SEQ ID NO: 4 - protein, 17 aa, Artificial Sequence
  Description of Artificial Sequence Synthetic peptide
WINTYTGETTYADDFKG

SEQ ID NO: 5 - protein, 6 aa, Artificial Sequence
  Description of Artificial Sequence Synthetic peptide
EGGVNN

SEQ ID NO: 6 - DNA, 714 bp, Artificial Sequence
  Description of Artificial Sequence Synthetic polynucleotide
atgaagaagaatattgcgttcctacttgcctctatgtttgtcttttctatagctacaaacgcgtatgctgatatccaggtgacccagtctccatcctccctgtctgcatctgtaggagaccgcgtcaccatcacttgcattaccagcactgatattgatgatgatatgaactggtatcagcagaaaccagggaaagttcctaagctcctgatctctggaggcaatactcttcgtcctggggtcccatctcggttcagtggcagtggatctgggacagatttcactctcaccatcagcagcctgcagcctgaagatgttgcaacttattactgtttgcaaagtgattctttgccgtacacgtttggccagggtaccaaggtggagatcaaacgaactgtggctgcaccatctgtcttcatcttcccgccatctgatgagcagttgaaatctggaactgcttctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaa

SEQ ID NO: 7 - protein, 214 aa, Artificial Sequence
  Description of Artificial Sequence Synthetic polypeptide
DIQVTQSPSSLSASVGDRVTITCITSTDIDDDMNWYQQKPGKVPKLLISGGNTLRPGVPSRFSGSGSGTDFTLTISSLQPEDVATYYCLQSDSLPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 11 aa, Artificial Sequence
  Description of Artificial Sequence Synthetic peptide
ITSTDIDDDMN

SEQ ID NO: 9 - protein, 7 aa, Artificial Sequence
  Description of Artificial Sequence Synthetic peptide
GGNTLRP

SEQ ID NO: 10 - protein, 9 aa, Artificial Sequence
  Description of Artificial Sequence Synthetic peptide
LQSDSLPYT

SEQ ID NO: 11 - protein, 6 aa, Artificial Sequence
  Description of Artificial Sequence Synthetic peptide
CDKTHT

SEQ ID NO: 12 - protein, 6 aa, Artificial Sequence
  Description of Artificial Sequence Synthetic peptide
CDKTHL

SEQ ID NO: 13 - protein, 5 aa, Artificial Sequence
  Description of Artificial Sequence Synthetic peptide
CDKTH

SEQ ID NO: 14 - protein, 4 aa, Artificial Sequence
  Description of Artificial Sequence Synthetic peptide
CDKT

SEQ ID NO: 15 - protein, 3 aa, Artificial Sequence
  Description of Artificial Sequence Synthetic peptide
CDK

SEQ ID NO: 16 - protein, 7 aa, Artificial Sequence
  Description of Artificial Sequence Synthetic peptide
CDKTHTC

SEQ ID NO: 17 - protein, 10 aa, Artificial Sequence
  Description of Artificial Sequence Synthetic peptide
CDKTHTCPPC

SEQ ID NO: 18 - protein, 10 aa, Artificial Sequence
  Description of Artificial Sequence Synthetic peptide
CDKTHTCPPS

SEQ ID NO: 19 - protein, 10 aa, Artificial Sequence
  Description of Artificial Sequence Synthetic peptide
CDKTHTSPPC

SEQ ID NO: 20 - protein, 10 aa, Artificial Sequence
  Description of Artificial Sequence Synthetic peptide
CDKTHTAPPC

SEQ ID NO: 21 - protein, 10 aa, Artificial Sequence
  Description of Artificial Sequence Synthetic peptide
CDKTHTSGGC

SEQ ID NO: 22 - protein, 6 aa, Artificial Sequence
  Description of Artificial Sequence Synthetic peptide
CYGPPC

SEQ ID NO: 23 - protein, 4 aa, Artificial Sequence
  Description of Artificial Sequence Synthetic peptide
VERK

SEQ ID NO: 24 - protein, 5 aa, Artificial Sequence
  Description of Artificial Sequence Synthetic peptide
VERKC

SEQ ID NO: 25 - protein, 6 aa, Artificial Sequence
  Description of Artificial Sequence Synthetic peptide
KYGPPC

SEQ ID NO: 26 - protein, 5 aa, Artificial Sequence
  Description of Artificial Sequence Synthetic peptide
KYGPP

SEQ ID NO: 27 - protein, 4 aa, Artificial Sequence
  Description of Artificial Sequence Synthetic peptide
KYGP

SEQ ID NO: 28 - protein, 3 aa, Artificial Sequence
  Description of Artificial Sequence Synthetic peptide
KYG

SEQ ID NO: 29 - protein, 107 aa, Artificial Sequence [sequence omitted]

SEQ ID NO: 30 - protein, 108 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 31 - protein, 108 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 32 - protein, 109 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTC

SEQ ID NO: 33 - protein, 112 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPC

SEQ ID NO: 34 - protein, 112 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPS

SEQ ID NO: 35 - protein, 112 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSPPC

SEQ ID NO: 36 - protein, 112 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTAPPC

SEQ ID NO: 37 - protein, 112 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTSGGC

SEQ ID NO: 38 - protein, 108 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCYGPPC

SEQ ID NO: 39 - protein, 107 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 40 - protein, 106 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKT

SEQ ID NO: 41 - protein, 105 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDK

SEQ ID NO: 42 - protein, 104 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCD

SEQ ID NO: 43 - protein, 101 aa, Artificial Sequence
  Description of Artificial Sequence Synthetic polypeptide
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERK

SEQ ID NO: 44 - protein, 102 aa, Artificial Sequence
  Description of Artificial Sequence Synthetic polypeptide
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKC

SEQ ID NO: 45 - protein, 106 aa, Artificial Sequence
  Description of Artificial Sequence Synthetic polypeptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPC

SEQ ID NO: 46 - protein, 105 aa, Artificial Sequence
  Description of Artificial Sequence Synthetic polypeptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPP

SEQ ID NO: 47 - protein, 103 aa, Artificial Sequence
  Description of Artificial Sequence Synthetic polypeptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYG

SEQ ID NO: 48 - protein, 102 aa, Artificial Sequence
  Description of Artificial Sequence Synthetic polypeptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKY

SEQ ID NO: 49 - protein, 101 aa, Artificial Sequence
  Description of Artificial Sequence Synthetic polypeptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESK

SEQ ID NO: 50 - protein, 104 aa, Artificial Sequence
  Description of Artificial Sequence Synthetic polypeptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGP

SEQ ID NO: 51 - protein, 108 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHX

SEQ ID NO: 52 - protein, 6 aa, Artificial Sequence
  Description of Artificial Sequence Synthetic peptide
CDKTHX

SEQ ID NO: 53 - protein, 4 aa, Artificial Sequence
  Description of Artificial Sequence Synthetic peptide
CPPC

SEQ ID NO: 54 - protein, 25 aa, Artificial Sequence
  Description of Artificial Sequence Synthetic peptide
EVQLVQSGPELKKPGASVKVSCKAS

SEQ ID NO: 55 - protein, 14 aa, Artificial Sequence
  Description of Artificial Sequence Synthetic peptide
WVRQAPGQGLEWMG

SEQ ID NO: 56 - protein, 32 aa, Artificial Sequence
  Description of Artificial Sequence Synthetic polypeptide
RFVFSLDTSVSTAYLQISSLKAEDTAVYYCER

SEQ ID NO: 57 - protein, 11 aa, Artificial Sequence
  Description of Artificial Sequence Synthetic peptide
WGQGTLVTVSS

SEQ ID NO: 58 - protein, 23 aa, Artificial Sequence
  Description of Artificial Sequence Synthetic peptide
DIQVTQSPSSLSASVGDRVTITC

SEQ ID NO: 59 - protein, 15 aa, Artificial Sequence
  Description of Artificial Sequence Synthetic peptide
WYQQKPGKVPKLLIS

SEQ ID NO: 60 - protein, 32 aa, Artificial Sequence
  Description of Artificial Sequence Synthetic polypeptide
GVPSRFSGSGSGTDFTLTISSLQPEDVATYYC

SEQ ID NO: 61 - protein, 10 aa, Artificial Sequence
  Description of Artificial Sequence Synthetic peptide
FGQGTKVEIK

SEQ ID NO: 62 - protein, 11 aa, Homo sapiens
RASQGISNYLA

SEQ ID NO: 63 - protein, 11 aa, Artificial Sequence
  Description of Artificial Sequence Synthetic peptide
ITSTSIDDDMN

SEQ ID NO: 64 - protein, 11 aa, Artificial Sequence
  Description of Artificial Sequence Synthetic peptide
ITSTDIEDDMN

SEQ ID NO: 65 - protein, 11 aa, Artificial Sequence
  Description of Artificial Sequence Synthetic peptide
ITSTDIDSDMN

SEQ ID NO: 66 - protein, 11 aa, Artificial Sequence
  Description of Artificial Sequence Synthetic peptide
ITSTDIDDSMN

SEQ ID NO: 67 - protein, 11 aa, Artificial Sequence
  Description of Artificial Sequence Synthetic peptide
ITSTSIDSSMN

SEQ ID NO: 68 - protein, 11 aa, Artificial Sequence
  Description of Artificial Sequence Synthetic peptide
ITSTDIESDMN

SEQ ID NO: 69 - protein, 11 aa, Artificial Sequence
  Description of Artificial Sequence Synthetic peptide
ITSTSIESDMN

SEQ ID NO: 70 - protein, 11 aa, Artificial Sequence
  Description of Artificial Sequence Synthetic peptide
ITSTSIESDMS

SEQ ID NO: 71 - protein, 7 aa, Homo sapiens
AASTLQS

SEQ ID NO: 72 - protein, 9 aa, Homo sapiens
QKYNSAPYT

SEQ ID NO: 73 - protein, 9 aa, Artificial Sequence
  Description of Artificial Sequence Synthetic peptide
LQSESLPYT

SEQ ID NO: 74 - protein, 5 aa, Artificial Sequence
  Description of Artificial Sequence Synthetic peptide
NYGMN

SEQ ID NO: 75 - protein, 5 aa, Homo sapiens
SYAMN

SEQ ID NO: 76 - protein, 17 aa, Homo sapiens
WINTNTGNPTYAQGFTG

SEQ ID NO: 77 - protein, 17 aa, Artificial Sequence
  Description of Artificial Sequence Synthetic peptide
WINTYTGETTYAEDFKG

SEQ ID NO: 78 - protein, 17 aa, Artificial Sequence
  Description of Artificial Sequence Synthetic peptide
WISTYTGETTYAEDFKG

SEQ ID NO: 79 - protein, 6 aa, Homo sapiens
EGYFDY

SEQ ID NO: 80 - protein, 6 aa, Artificial Sequence
  Description of Artificial Sequence Synthetic peptide
EGGVDN

SEQ ID NO: 81 - protein, 6 aa, Artificial Sequence
  Description of Artificial Sequence Synthetic peptide
EGGVQN

SEQ ID NO: 82 - protein, 6 aa, Artificial Sequence
  Description of Artificial Sequence Synthetic peptide
EGGVSN

SEQ ID NO: 83 - protein, 107 aa, Artificial Sequence [sequence omitted]

SEQ ID NO: 84 - protein, 107 aa, Homo sapiens
DIQMTQSPSSLSASVGDRVTITCRASQGISNYLAWYQQKPGKVPKLLIYAASTLQSGVPSRFSGSGSGTDFTLTISSLQPEDVATYYCQKYNSAPYTFGQGTKLEIK

SEQ ID NO: 85 - protein, 107 aa, Artificial Sequence
  Description of Artificial Sequence Synthetic polypeptide
DIQVTQSPSSLSASVGDRVTITCITSTSIDDDMNWYQQKPGKVPKLLISGGNTLRPGVPSRFSGSGSGTDFTLTISSLQPEDVATYYCLQSDSLPYTFGQGTKVEIK

SEQ ID NO: 86 - protein, 107 aa, Artificial Sequence
  Description of Artificial Sequence Synthetic polypeptide
DIQVTQSPSSLSASVGDRVTITCITSTDIEDDMNWYQQKPGKVPKLLISGGNTLRPGVPSRFSGSGSGTDFTLTISSLQPEDVATYYCLQSDSLPYTFGQGTKVEIK

SEQ ID NO: 87 - protein, 107 aa, Artificial Sequence
  Description of Artificial Sequence Synthetic polypeptide
DIQVTQSPSSLSASVGDRVTITCITSTDIDSDMNWYQQKPGKVPKLLISGGNTLRPGVPSRFSGSGSGTDFTLTISSLQPEDVATYYCLQSDSLPYTFGQGTKVEIK

SEQ ID NO: 88 - protein, 107 aa, Artificial Sequence
  Description of Artificial Sequence Synthetic polypeptide
DIQVTQSPSSLSASVGDRVTITCITSTDIDDSMNWYQQKPGKVPKLLISGGNTLRPGVPSRFSGSGSGTDFTLTISSLQPEDVATYYCLQSDSLPYTFGQGTKVEIK

SEQ ID NO: 89 - protein, 107 aa, Artificial Sequence
  Description of Artificial Sequence Synthetic polypeptide
DIQVTQSPSSLSASVGDRVTITCITSTSIDSSMNWYQQKPGKVPKLLISGGNTLRPGVPSRFSGSGSGTDFTLTISSLQPEDVATYYCLQSDSLPYTFGQGTKVEIK

SEQ ID NO: 90 - protein, 107 aa, Artificial Sequence
  Description of Artificial Sequence Synthetic polypeptide
DIQVTQSPSSLSASVGDRVTITCITSTDIESDMNWYQQKPGKVPKLLISGGNTLRPGVPSRFSGSGSGTDFTLTISSLQPEDVATYYCLQSDSLPYTFGQGTKVEIK

SEQ ID NO: 91 - protein, 107 aa, Artificial Sequence
  Description of Artificial Sequence Synthetic polypeptide
DIQVTQSPSSLSASVGDRVTITCITSTDIESDMNWYQQKPGKVPKLLISGGNTLRPGVPSRFSGSGSGTDFTLTISSLQPEDVATYYCLQSESLPYTFGQGTKVEIK

SEQ ID NO: 92 - protein, 107 aa, Artificial Sequence
  Description of Artificial Sequence Synthetic polypeptide
DIQVTQSPSSLSASVGDRVTITCITSTSIESDMNWYQQKPGKVPKLLISGGNTLRPGVPSRFSGSGSGTDFTLTISSLQPEDVATYYCLQSDSLPYTFGQGTKVEIK

SEQ ID NO: 93 - protein, 107 aa, Artificial Sequence
  Description of Artificial Sequence Synthetic polypeptide
DIQVTQSPSSLSASVGDRVTITCITSTSIESDMSWYQQKPGKVPKLLISGGNTLRPGVPSRFSGSGSGTDFTLTISSLQPEDVATYYCLQSDSLPYTFGQGTKVEIK

SEQ ID NO: 94 - protein, 107 aa, Artificial Sequence
  Description of Artificial Sequence Synthetic polypeptide
DIQVTQSPSSLSASVGDRVTITCITSTSIESDMNWYQQKPGKVPKLLISGGNTLRPGVPSRFSGSGSGTDFTLTISSLQPEDVATYYCLQSESLPYTFGQGTKVEIK

SEQ ID NO: 95 - protein, 115 aa, Artificial Sequence [sequence omitted]

SEQ ID NO: 96 - protein, 115 aa, Homo sapiens
QVQLVQSGSELKKPGASVKVSCKASGYTFTSYAMNWVRQAPGQGLEWMGWINTNTGNPTYAQGFTGRFVFSLDTSVSTAYLQISSLKAEDTAVYYCAREGYFDYWGQGTLVTVSS

SEQ ID NO: 97 - protein, 115 aa, Artificial Sequence
  Description of Artificial Sequence Synthetic polypeptide
EVQLVQSGPELKKPGASVKVSCKASGYTFTNYGMNWVRQAPGQGLEWMGWINTYTGETTYAEDFKGRFVFSLDTSVSTAYLQISSLKAEDTAVYYCEREGGVNNWGQGTLVTVSS

SEQ ID NO: 98 - protein, 115 aa, Artificial Sequence
  Description of Artificial Sequence Synthetic polypeptide
EVQLVQSGPELKKPGASVKVSCKASGYTFTNYGMNWVRQAPGQGLEWMGWISTYTGETTYAEDFKGRFVFSLDTSVSTAYLQISSLKAEDTAVYYCEREGGVNNWGQGTLVTVSS

SEQ ID NO: 99 - protein, 115 aa, Artificial Sequence
  Description of Artificial Sequence Synthetic polypeptide
EVQLVQSGPELKKPGASVKVSCKASGYTFTNYGMNWVRQAPGQGLEWMGWINTYTGETTYAEDFKGRFVFSLDTSVSTAYLQISSLKAEDTAVYYCEREGGVDNWGQGTLVTVSS

SEQ ID NO: 100 - protein, 115 aa, Artificial Sequence
  Description of Artificial Sequence Synthetic polypeptide
EVQLVQSGPELKKPGASVKVSCKASGYTFTNYGMNWVRQAPGQGLEWMGWINTYTGETTYAEDFKGRFVFSLDTSVSTAYLQISSLKAEDTAVYYCEREGGVQNWGQGTLVTVSS

SEQ ID NO: 101 - protein, 115 aa, Artificial Sequence
  Description of Artificial Sequence Synthetic polypeptide
EVQLVQSGPELKKPGASVKVSCKASGYTFTNYGMNWVRQAPGQGLEWMGWINTYTGETTYAEDFKGRFVFSLDTSVSTAYLQISSLKAEDTAVYYCEREGGVSNWGQGTLVTVSS